(19) United States
(12) Patent Application Publication
(10) Pub. No.: US 2013/0251695 A1
(43) Pub. Date: Sep. 26, 2013

(54) Cosmetic Compositions

(71) Applicant: GANEDEN BIOTECH, INC., Mayfield Heights, OH (US)

(72) Inventors: Sean Farmer, Miami, FL (US); Michael A. Bush, Brecksville, OH (US); David Keller, University Heights, OH (US); Andrew R. Lefkowitz, Mayfield Heights, OH (US)

(21) Appl. No.: 13/781,001

(22) Filed: Feb. 28, 2013

Related U.S. Application Data
(60) Provisional application No. 61/604,493, filed on Feb. 28, 2012.
(60) Provisional application No. 61/608,466, filed on Mar. 8, 2012.
(60) Provisional application No. 61/709,678, filed on Oct. 4, 2012.
(60) Provisional application No. 61/712,375, filed on Oct. 11, 2012.

(51) Int. Cl. A61K 8/99; A61Q 19/08

(52) U.S. Cl. CPC A61K 8/99; A61Q 19/08
USPC 424/93.46; 424/780

(57) ABSTRACT

The present application relates to the use of lactic acid-producing bacteria and the extracellular product thereof in cosmetic compositions.

### RELATED APPLICATIONS

This application claims the benefit of priority under 35 U.S.C. §119(e) to U.S. Provisional Application No. 61/604,493, filed Feb. 28, 2012, U.S. Provisional Application No. 61/608,466, filed Mar. 8, 2012, U.S. Provisional Application No. 61/709,678, filed Oct. 4, 2012, and to U.S. Provisional Application No. 61/712,375, filed Oct. 11, 2012. Each of these applications is incorporated herein by reference in its entirety.

### FIELD OF THE INVENTION

The present application relates to the use of lactic acid-producing bacteria in cosmetic compositions.

### BACKGROUND OF THE INVENTION

Probiotic organisms are non-pathogenic, non-toxigenic, and retain viability during storage. Since probiotics do not generally permanently colonize the host, they need to be administered regularly for any health promoting properties to persist.

### SUMMARY OF THE INVENTION

The invention features a topical composition for the reduction of visible signs of aging comprising an extracellular product of Bacillus coagulans and a dermatologically acceptable carrier. Preferably, the Bacillus coagulans comprises GBI-30 (ATCC Designation Number PTA-6086). The extracellular product comprises a liquid culture supernatant, is in the form of a dried powder, or is in the form of a reconstituted liquid from the dried powder. Suitable forms of the composition include an emulsion, a lotion, a cream, an oil, an ointment, a suspension, a gel, a powder, an aerosol powder, a scrub, a mask, an aerosol spray, a semi-solid formulation, a shampoo, and a conditioner.

In one aspect, the active agents are combined with a carrier or excipient that is physiologically compatible with the dermal or epithelial tissue of a human or animal to which it is administered. Suitable dermatologically acceptable carriers include hydrocarbon oils and waxes, silicone oils, vegetable, animal or marine fats or oils, glyceride derivatives, fatty acids or fatty acid esters or alcohols or alcohol ethers, lecithin, lanolin and derivatives, polyhydric alcohols or esters, wax esters, sterols and phospholipids.

In one example, the supernatant comprises at least about 1% by volume of the composition, e.g., at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% by volume of the composition. In another example, the supernatant (e.g., dried supernatant) comprises at least about 1% by weight of the composition, e.g., at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% by weight of the composition.

In some cases, the compositions of the invention comprise an aging-reducing amount, i.e., an anti-aging amount, of the extracellular product of Bacillus coagulans bacterium. In another example, the extracellular product of Bacillus coagulans is present in an inflammation-reducing amount. For example, the extracellular product of Bacillus coagulans comprises between 1 μL and 100 L, e.g., between 10 μL and 10 L; between 100 μL and 1 L; between 1 mL and 100 mL; or about 10 mL. Alternatively, an anti-aging amount of the compositions of the invention comprises an amount that improves hydration/moisturization of treated skin, as measured by, e.g., Nova DPM 9003 (Gloucester, Mass.) by between 1% and 95%, as compared to a pre-treatment baseline level; improves skin elasticity/flexibility, as measured by, e.g., Cutometer SEM 575 (Courage+Khazaka Electronic GmbH, Köln, Germany), by between 1% and 95%, as compared to a pre-treatment baseline; reduces fine lines and wrinkles, as measured by e.g., Visioscan® VC 98, (Courage+Khazaka Electronic GmbH, Köln, Germany), by between 1% and 95%, as compared to a pre-treatment baseline level; reduces under eye, as determined by, e.g., photographic evaluation utilizing the R.W. Johnson Pharmaceutical Research Institute descriptive scale (Griffiths et al., 1992 Arch Dermatol, 128(3): 347-351, incorporated herein by reference), by between 1% and 95%, as compared to a pre-treatment baseline level; reduces under eye dark circles, as determined by, e.g., photographic evaluation utilizing the R.W. Johnson Pharmaceutical Research Institute descriptive scale (Griffiths et al., 1992 Arch Dermatol, 128(3): 347-351, incorporated herein by reference), by between 1% and 95%, as compared to a pre-treatment baseline level; reduces skin inflammation, as determined by, e.g., photographic evaluation, by between 1% and 95%, as compared to a pre-treatment baseline level; decreases skin pore size, as determined by, e.g., photographic evaluation, by between 1% and 95%, as compared to a pre-treatment baseline level; decreases skin roughness, as measured by e.g., Visioscan® VC 98, (Courage+Khazaka Electronic GmbH, Köln, Germany), by between 1% and 95%, as compared to a pre-treatment baseline level; or decreases skin redness, as determined by, e.g., photographic evaluation, by between 1% and 95%, as compared to a pre-treatment baseline level.

The extracellular product of Bacillus coagulans bacterium comprises compounds, e.g., anti-inflammatory or anti-aging compounds, between 3 kDa and 200 k Da, e.g., compounds less than 3 kDa; compounds between 3 kDa and 30 kDa; compounds between 30 kDa and 200 kDa; and compounds between 25 and 75 kDa.

Optionally, the composition further comprises from about 0.1% to about 10% by weight of a penetration enhancer selected from the group consisting of sulfoxides, alcohols, polyols, alkanes, fatty acids, esters, amines and amides, terpenes, surface-active agents, cyclodextrins, and mixtures thereof. In some cases, the extracellular product of Bacillus coagulans is lyophilized or is in the form of a reconstituted liquid from dried powder, i.e., the extracellular product is dried (e.g., freeze-dried, vacuum dried, air dried, or dried by application of heat) and subsequently reconstituted.

An exemplary formulation comprising Bacillus coagulans extracellular product includes the following ingredients: Bacillus coagulans extracellular product, water, isopropyl myristate, isocetyl stearate, glycerin, ricinus communis (castor) seed oil, hydrogenated vegetable oil, vegetable oil, hydrogenated castor oil, cetyl alcohol, polyacrylamide, c13-14 isoparaffin, laureth-7, ethylhexyl methoxycinnamate, squalene, laneth-16, ceteth-16, oleth-16, steareth-16, caprylyl glycol, phenoxyethanol, hexylene glycol, and fragrance.

Drying and reconstituting Bacillus coagulans extracellular product (metabolites/supernatant) in, e.g., saline, results in unexpected anti-inflammatory effects. For example, drying the Bacillus coagulans extracellular product (metabolites/supernatant) inactivates or removes undesirable compounds (e.g., volatile organic compounds <30 kDa) that would otherwise inhibit the anti-inflammatory effects of the Bacillus coagulans extracellular product prior to drying and rehydration. In some cases, the dried and reconstituted Bacillus coagulans extracellular product is spray-dried to remove the undesirable compounds. Exemplary components present in the Bacillus coagulans extracellular product include peptidoglycan from lysed cell walls and/or lipoteichoic acid (LTA).

Culture supernatants that are dried and subsequently rehydrated are useful in products where conditions are not optimal for transportation of large volumes of liquid. Specifically, culture supernatants that are dried and subsequently rehydrated are useful in any situation where an anti-inflammatory effect is desired. For example, dried and reconstituted Bacillus coagulans extracellular product contains 30-200 kDa compounds that reduce migration of inflammatory cells (e.g., leukocytes, phagocytes, monocytes, lymphocytes, and polymorphonuclear leukocytes (PMNs)) and induce the production of anti-inflammatory cytokines (e.g., interleukin-4 (IL-4), IL-6, and tumor necrosis factor alpha (TNFα)).

A method for the topical treatment or reduction of visible signs of aging in a subject is carried out by topically applying to affected skin the composition described above. In some cases, the skin to be treated is not characterized by a pathologic microbial infection such as an infection by a pathologic virus (e.g., Herpes simplex viruses I and II), yeast (e.g., Candida albicans and C. tropicalis), fungus (e.g., Trichophyton mentagrophytes, T. interdigitale, and T. rubrum, and T. yaoundei), or bacteria (e.g., Staphylococcus aureus, S. epidermidis, Streptococcus pyogenes, Pseudomonas aeruginosa, Escherichia coli (enterohemorragic species), Clostridium perfingens, C. Gardnerella vaginalis, Propionibacterium acnes, Aeromonas hydrophilia, Aspergillus species, Proteus species, and Klebsiella species), i.e., the skin does not comprise a dermal pathogen. The method leads to a surprising reduction in visible signs of aging after at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days of treatment.

For example, hydration/moisturization of treated skin is improved, as measured by, e.g., Nova DPM 9003 (Gloucester, Mass.) by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Skin elasticity/flexibility is improved, as measured by, e.g., Cutometer SEM 575 (Courage+Khazaka Electronic GmbH, Köln, Germany), by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline. Fine lines and wrinkles are reduced, as measured by e.g., Visioscan® VC 98, (Courage+Khazaka Electronic GmbH, Köln, Germany), by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Under eye puffiness is reduced, as determined by, e.g., photographic evaluation utilizing the R.W. Johnson Pharmaceutical Research Institute descriptive scale (Griffiths et al., 1992 Arch Dermatol, 128(3): 347-351, incorporated herein by reference), by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Under eye dark circles are reduced, as determined by, e.g., photographic evaluation utilizing the R.W. Johnson Pharmaceutical Research Institute descriptive scale (Griffiths et al., 1992 Arch Dermatol, 128(3): 347-351, incorporated herein by reference), by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Skin inflammation is reduced, as determined by, e.g., photographic evaluation, by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Skin pore size is decreased, as determined by, e.g., photographic evaluation, by at least at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Skin roughness is decreased, as measured by e.g., Visioscan® VC 98, (Courage+Khazaka Electronic GmbH, Köln, Germany), by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Finally, skin redness is decreased, as determined by, e.g., photographic evaluation, by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level.

For example, as described in the examples below, at 4 weeks, the cream plus supernatant increases skin hydration by 7.13% more than a placebo cream. In another example, the cream plus Bacillus coagulans supernatant increases skin moisturization by 19.05%. As described in the examples below, the cream plus supernatant increases skin elasticity by 3.11% more than a placebo cream. As described in the examples below, the cream plus supernatant decreases the number of coarse skin lines by 20.57% more than a placebo cream. In another example, the cream plus Bacillus coagulans supernatant decreases skin roughness by 19.44%. In another example, the cream plus supernatant increases skin smoothness by 4.33% more than a placebo cream. In yet another example, the cream plus supernatant decreases skin shadows by 7.09% more than a placebo cream. As described in the examples below, on visual evaluation, the cream plus supernatant results in a 17% increase in the number of subjects showing improvement of eye area fine lines and wrinkles more than a placebo cream. In another example, the cream plus Bacillus coagulans supernatant decreases skin wrinkle and fine lines by 71.50% compared to only a 12.79% reduction with placebo cream. Finally, as described in the examples below, the cream plus supernatant results in an 8.33% increase in the number of subjects showing improvement of under eye puffiness more than a placebo cream.

The subject is preferably a mammal in need of such treatment, e.g., a subject that has visible signs of aging or a predisposition thereto. For example, the subject is identified as suffering from visible signs of aging or a predisposition thereto by detecting a sign or symptom selected from the group consisting of fine lines or wrinkles around the eye area, under-eye puffiness, dark under-eye circles, rough skin, reduced skin hydration/moisturization, and reduced skin elasticity/flexibility. The mammal can be, e.g., any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

In some cases, the compositions of the invention comprise an aging-reducing amount, i.e., an anti-aging amount, of the extracellular product of Bacillus coagulans bacterium. In another example, the extracellular product of Bacillus coagulans is present in an inflammation-reducing amount. For example, the extracellular product of Bacillus coagulans comprises between 1 μL and 100 L, e.g., between 10 μL and 10 L; between 100 μL and 1 L; between 1 mL and 100 mL; or about 10 mL. In some cases, the extracellular product of Bacillus coagulans is lyophilized. In other cases, the extracellular product of Bacillus coagulans is dried (e.g., freeze-dried, vacuum dried, or air dried) and reconstituted.

Exemplary bacterial species for the compositions and methods described herein include Bacillus coagulans, e.g., Bacillus coagulans hammer, preferably Bacillus coagulans hammer strain Accession No. ATCC 31284, or one or more strains derived from Bacillus coagulans hammer strain Accession No. ATCC 31284 (e.g., ATCC Numbers: GBI-20 (GB-20), ATCC Designation Number PTA-6085; GBI-30 (GB-30/Ganeden BC³⁰™/BC³⁰), ATCC Designation Number PTA-6086; and GBI-40 (GB-40), ATCC Designation Number PTA-6087; see, U.S. Pat. No. 6,849,256 to Farmer). Preferably, the Bacillus coagulans comprises GBI-30 (BC³⁰), or any strain of the organism described in U.S. Ser. No. 11/706,642, hereby incorporated by reference. The Bacillus coagulans Hammer strains of the invention are non-pathogenic and generally regarded as safe for use in human nutrition (i.e., GRAS classification) by the U.S. Federal Drug Administration (FDA) and the U.S. Department of Agriculture (USDA), and by those skilled in the art.

Exemplary formulations of the compositions of the invention include an emulsion, a lotion, a cream, an oil, an ointment, a suspension, a gel, a powder, an aerosol powder, a scrub, a mask, an aerosol spray, a semi-solid formulation, a shampoo, and a conditioner. The compositions of the invention are administered topically, e.g., to the skin. The compositions are administered at least once per day, e.g., at least twice per day, at least 3 times per day, at least 4 times per day, or at least 5 times per day. Preferably, the compositions are administered for at least 24 hours, at least 48 hours, at least 72 hours, or for at least 7 days, at least 14 days, at least 28 days, at least 30 days, at least 60 days, at least 90 days, or for at least 4 months, at least 6 months, at least 9 months, or for at least 1 year, at least 2 years, or at least 3 years.

Drying and reconstituting Bacillus coagulans extracellular product (metabolites/supernatant) in, e.g., saline, results in unexpected anti-inflammatory effects. Drying the Bacillus coagulans extracellular product (metabolites/supernatant) inactivates or removes undesirable compounds (e.g., volatile organic compounds <30 kDa) that would otherwise inhibit the anti-inflammatory effects of the Bacillus coagulans extracellular product prior to drying and rehydration. For example, the dried and reconstituted Bacillus coagulans extracellular product is spray-dried to remove the undesirable compounds. In some cases, components present in the Bacillus coagulans extracellular product include peptidoglycan from lysed cell walls and/or lipoteichoic acid (LTA).

Culture supernatants that are dried and subsequently rehydrated are useful in products where conditions are not optimal for transportation of large volumes of liquid. Specifically, culture supernatants that are dried and subsequently rehydrated are useful in any situation where an anti-inflammatory effect is desired. For example, dried and reconstituted Bacillus coagulans extracellular product contains 30-200 kDa compounds that reduce migration of inflammatory cells (e.g., leukocytes, phagocytes, monocytes, lymphocytes, and polymorphonuclear leukocytes (PMNs)) and induce the production of anti-inflammatory cytokines (e.g., interleukin-4 (IL-4), IL-6, and tumor necrosis factor alpha (TNFα)).

Dried and reconstituted Bacillus coagulans extracellular product is also useful in the various cosmetic, e.g., anti-aging, products described herein, which increase skin hydration/moisturization, increase skin elasticity, reduce the appearance of rough skin, fine lines, and wrinkles, reduce the appearance of under eye puffiness/under eye dark circles, and reduce skin inflammation. Dried and reconstituted Bacillus coagulans extracellular product is also useful in topical formulations designed to inhibit the growth of bacteria, fungus, yeast, and mycotic pathogens, thereby improving local skin flora.

For example, drying and reconstituting the Bacillus coagulans extracellular product results in at least 1% greater anti-inflammatory or anti-aging activity compared to Bacillus coagulans extracellular product alone, e.g., at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 99% greater anti-inflammatory or anti-aging activity compared to Bacillus coagulans extracellular product alone.

Also provided is a composition comprising a dry powder comprising acellular culture supernatant of Bacillus coagulans in a eukaryotic tissue culture medium. By “acellular culture supernatant” is meant a culture supernatant that is substantially free of cell walls, cell wall fragments, and other cellular components. For example, the cells are separated from the culture supernatant by a centrifuge. In some cases, the medium is serum free medium. Suitable media include Roswell Park Memorial Institute (RPMI)-1640 medium, Dulbecco's modified eagle medium (DMEM), Eagle's minimal essential medium (EMEM), minimal essential medium (MEM), Iscove's modified Dulbecco's media (IMDM), Ham's medium, minimal essential medium alpha (AMEM), Glasgow minimal essential medium (GMEM), and Hank's balanced salt solution medium (HBSS).

Other suitable media are described in U.S. Pat. No. 6,383,810 (incorporated herein by reference), and include MCDB 131, MCDB 153, MDEM, M199, McCoy's 5A, Williams' Media E, Leibovitz's L-15 Medium, Grace's Insect Medium, IPL-41 Insect Medium, TC-100 Insect Medium, Schneider's Drosophila Medium, Wolf & Quimby's Amphibian Culture Medium, cell-specific serum-free media (SFM) such as those designed to support the culture of keratinocytes, endothelial cells, hepatocytes, melanocytes, etc., F10 Nutrient Mixture and F12 Nutrient Mixture. Other media, media supplements, and media subgroups suitable for preparation by the invention are available commercially (e.g., from Life Technologies, Inc.™; Rockville, Md., and Sigma-Aldrich®; St. Louis, Mo.). Formulations for these media, media supplements and media subgroups, as well as many other commonly used animal cell culture media, media supplements and media subgroups are well-known in the art and may be found, for example in the GIBCO/BRL Catalogue and Reference Guide (Life Technologies, Inc.™; Rockville, Md.) and in the Sigma-Aldrich® Cell Catalogue (Sigma; St. Louis, Mo.).

Also provided is a topical composition for the reduction of visible signs of aging comprising an isolated Bacillus coagulans bacterium and a dermatologically acceptable carrier. For example, the compositions of the invention comprise an aging-reducing amount, i.e., an anti-aging amount of an isolated Bacillus coagulans bacterium itself. In another example, the Bacillus coagulans is present in an inflammation-reducing amount. For example, an anti-aging amount of the Bacillus coagulans comprises between 0.1 mg and 10 grams, e.g., about 1 mg to about 10 grams, about 10 mg to about 5 grams; about 100 mg to about 2 gram; or about 200 mg to about 1 gram. Alternatively, an anti-aging amount of the Bacillus coagulans comprises an amount that improves hydration/moisturization of treated skin, as measured by, e.g., Nova DPM 9003 (Gloucester, Mass.) by between 1% and 95%, as compared to a pre-treatment baseline level; improves skin elasticity/flexibility, as measured by, e.g., Cutometer SEM 575 (Courage+Khazaka Electronic GmbH, Köln, Germany), by between 1% and 95%, as compared to a pre-treatment baseline; reduces fine lines and wrinkles, as measured by e.g., Visioscan® VC 98, (Courage+Khazaka Electronic GmbH, Köln, Germany), by between 1% and 95%, as compared to a pre-treatment baseline level; reduces under eye, as determined by, e.g., photographic evaluation utilizing the R.W. Johnson Pharmaceutical Research Institute descriptive scale (Griffiths et al., 1992 Arch Dermatol, 128(3): 347-351, incorporated herein by reference), by between 1% and 95%, as compared to a pre-treatment baseline level; reduces under eye dark circles, as determined by, e.g., photographic evaluation utilizing the R.W. Johnson Pharmaceutical Research Institute descriptive scale (Griffiths et al., 1992 Arch Dermatol, 128(3): 347-351, incorporated herein by reference), by between 1% and 95%, as compared to a pre-treatment baseline level; reduces skin inflammation, as determined by, e.g., photographic evaluation, by between 1% and 95%, as compared to a pre-treatment baseline level; decreases skin pore size, as determined by, e.g., photographic evaluation, by between 1% and 95%, as compared to a pre-treatment baseline level; decreases skin roughness, as measured by e.g., Visioscan® VC 98, (Courage+Khazaka Electronic GmbH, Köln, Germany), by between 1% and 95%, as compared to a pre-treatment baseline level; or decreases skin redness, as determined by, e.g., photographic evaluation, by between 1% and 95%, as compared to a pre-treatment baseline level.

Any of a variety of methods for placing the bacterial composition into a composition can be used. However, preferred methods include a “spray-dry” method in which the compositions are exposed in a low humidity chamber to an atomized mix containing a liquid composition, where the chamber is subsequently exposed to approximately 80-110° F. to dry the liquid, thereby impregnating the material of composition with the components.

A typical concentration is from approximately 1×10⁷ to 1×10¹² colony forming units (CFU); 1×10⁸ to 1×10¹¹ CFU; or 1×10⁹ to 1×10¹⁰ CFU of viable bacterium or spores/g of composition. In one aspect, the amount of bacteria is about 10⁴ to 10¹⁴ CFU of bacteria per gram of probiotic composition (i.e., vegetative cells and/or bacterial spores), preferably 10⁵ to 10¹³ CFU/g of composition. Alternatively, the concentrations are 10⁸ to 10¹³ CFU/g; 10⁹ to 10¹² CFU/g; or 10¹⁰ to 10¹¹ CFU/g of composition. For example, the composition comprises about 1×10⁶, 2×10⁶, or 5×10⁷ CFU Bacillus coagulans bacteria (per gram of composition) in the form of spray-dried powder. The actual amount in a composition will vary depending upon the amounts of composition to be dispersed into the composition and upon routes of dispersal. Following drying, the composition is ready for immediate use or for storage in a sterile package.

The Bacillus coagulans bacterium comprises compounds, e.g., anti-inflammatory or anti-aging compounds, between 3 kDa and 200 k Da, e.g., compounds less than 3 kDa; compounds between 3 kDa and 30 kDa; and compounds between 30 kDa and 200 kDa.

The isolated Bacillus coagulans bacterium is in the form of a spore or a vegetative cell. In some cases, the isolated Bacillus coagulans is in the form of a spore. Alternatively, the isolated Bacillus coagulans is in the form of a vegetative cell. In another aspect, the isolated Bacillus coagulans is in the form of a mixture of vegetative cells and spores. The Bacillus coagulans is predominantly in spore form, e.g., about 75%, about 80%, about 85%, about 90%, about 95%, about 96%, about 97%, about 98%, about 99%, or about 100% spores. For example, the Bacillus coagulans comprises 99.9% spores. Alternatively, the Bacillus coagulans is predominantly in vegetative form, e.g., about 75%, about 80%, about 85%, about 90%, about 95%, about 99%, or about 100% vegetative cells. In some cases, the Bacillus coagulans bacterium is lyophilized.

The composition is in the form of an emulsion, a lotion, a cream, an oil, an ointment, a suspension, a gel, a powder, an aerosol powder, a scrub, a mask, an aerosol spray, a semi-solid formulation, a shampoo, or a conditioner. In some cases, the composition is in the form of a dried powder.

The Bacillus coagulans bacteria (e.g., spores or vegetative cells) comprise at least about 1% by volume of the composition, e.g., at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% by volume of the composition. For example, Bacillus coagulans in the form of dried powder comprises at least 1% by volume of the composition. In another example, the Bacillus coagulans bacteria (e.g., spores or vegetative cells) comprise at least about 1% by weight of the composition, e.g., at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% by weight of the composition. For example, Bacillus coagulans in the form of dried powder comprises at least 1% by weight of the composition.

The Bacillus coagulans bacterium is viable or non-viable. For example, the non-viable Bacillus coagulans bacterium is inactivated, irradiated, heat killed or dead.

Optionally, the composition further comprises from about 0.1% to about 10% by weight of a penetration enhancer selected from the group consisting of sulfoxides, alcohols, polyols, alkanes, fatty acids, esters, amines and amides, terpenes, surface-active agents, cyclodextrins, and mixtures thereof.

As stated above, exemplary bacterial species for the compositions and methods described herein include Bacillus coagulans, e.g., Bacillus coagulans hammer, preferably Bacillus coagulans hammer strain Accession No. ATCC 31284, or one or more strains derived from Bacillus coagulans hammer strain Accession No. ATCC 31284 (e.g., ATCC Numbers: GBI-20 (GB-20), ATCC Designation Number PTA-6085; GBI-30 (GB-30/Ganeden BC³⁰™/BC³⁰), ATCC Designation Number PTA-6086; and GBI-40 (GB-40), ATCC Designation Number PTA-6087; see, U.S. Pat. No. 6,849,256 to Farmer). Preferably, the Bacillus coagulans comprises GBI-30 (BC³⁰), or any strain of the organism described in U.S. Ser. No. 11/706,642, hereby incorporated by reference.

A method for the topical treatment or reduction of visible signs of aging in a subject is carried out by topically applying to affected skin a composition comprising an isolated Bacillus coagulans bacterium and a dermatologically acceptable carrier. As described in detail above, suitable Bacillus coagulans bacterium strains include GBI-30 strain (ATCC Designation Number PTA-6086), GBI-20 strain (ATCC Designation Number PTA-6085), and GBI-40 strain (ATCC Designation Number PTA-6087). In some cases, the skin to be treated is not characterized by a pathologic microbial infection such as an infection by a pathologic virus, yeast, fungus, or bacteria, i.e., the skin does not comprise a dermal pathogen. Alternatively, the composition inhibits the growth of pathogenic bacteria, fungus, or yeast. The method leads to a surprising reduction in visible signs of aging in a subject after at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days of treatment.

For example, hydration/moisturization of treated skin is improved, as measured by, e.g., Nova DPM 9003 (Gloucester, Mass.) by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Skin elasticity/flexibility is improved, as measured by, e.g., Cutometer SEM 575 (Courage+Khazaka Electronic GmbH, Köln, Germany), by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Fine lines and wrinkles, as measured by e.g., Visioscan® VC 98, (Courage+Khazaka Electronic GmbH, Köln, Germany), are reduced by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Under eye puffiness is reduced, as determined by, e.g., photographic evaluation utilizing the R.W. Johnson Pharmaceutical Research Institute descriptive scale (Griffiths et al., 1992 Arch Dermatol, 128(3): 347-351, incorporated herein by reference), by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Under eye dark circles are reduced, as determined by, e.g., photographic evaluation utilizing the R.W. Johnson Pharmaceutical Research Institute descriptive scale (Griffiths et al., 1992 Arch Dermatol, 128(3): 347-351, incorporated herein by reference), by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Skin inflammation is reduced, as determined by, e.g., photographic evaluation, by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Skin pore size is decreased, as determined by, e.g., photographic evaluation, by at least at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Skin roughness is decreased, as measured by e.g., Visioscan® VC 98, (Courage+Khazaka Electronic GmbH, Köln, Germany), by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level. Finally, skin redness is decreased, as determined by, e.g., photographic evaluation, by at least 1%, e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a pre-treatment baseline level.

The subject is preferably a mammal in need of such treatment, e.g., a subject that has visible signs of aging or a predisposition thereto. For example, the subject is identified as suffering from visible signs of aging or a predisposition thereto by detecting a sign or symptom selected from the group consisting of fine lines or wrinkles around the eye area, under-eye puffiness, dark under-eye circles, rough skin, reduced skin hydration/moisturization, and reduced skin elasticity. The mammal can be, e.g., any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

For example, the compositions of the invention comprise an aging-reducing amount, i.e., an anti-aging amount of an isolated Bacillus coagulans bacterium itself. In another example, the Bacillus coagulans is present in an inflammation-reducing amount. For example, the Bacillus coagulans comprises between 0.1 mg and 10 grams, e.g., about 1 mg to about 10 grams, about 10 mg to about 5 grams; about 100 mg to about 1 gram; or about 200 mg to about 1 gram. In some cases, the Bacillus coagulans bacterium is lyophilized. In other cases, the Bacillus coagulans is dried (e.g., freeze-dried, vacuum dried, or air dried) and reconstituted.

The composition is in the form of an emulsion, a lotion, a cream, an oil, an ointment, a suspension, a gel, a powder, an aerosol powder, a scrub, a mask, an aerosol spray, a semi-solid formulation, a shampoo, or a conditioner. In some cases, the composition is in the form of a dried powder.

The compositions are administered topically, e.g., to the skin. The compositions are administered at least once per day, e.g., at least twice per day, at least 3 times per day, at least 4 times per day, or at least 5 times per day. Preferably, the compositions are administered for at least 24 hours, at least 48 hours, at least 72 hours, or for at least 7 days, at least 14 days, at least 28 days, at least 30 days, at least 60 days, at least 90 days, or for at least 4 months, at least 6 months, at least 9 months, or for at least 1 year, at least 2 years, or at least 3 years.

The Bacillus coagulans bacterium is viable or non-viable. For example, the non-viable Bacillus coagulans bacterium is inactivated, irradiated, heat killed or dead.

Also provided are methods for topically reducing visible signs of a skin disorder in a subject by topically applying to affected skin a composition comprising an isolated Bacillus coagulans bacterium or an extracellular product thereof and a dermatologically acceptable carrier. For example, the skin disorder is acne. In some cases, the Bacillus coagulans extracellular product is dried and reconstituted.

Purified and/or isolated Bacillus coagulans or Bacillus coagulans extracellular product is particularly useful in the methods and compositions described herein. By “purified” or “substantially purified” is meant a Bacillus coagulans bacterium or Bacillus coagulans extracellular product that is substantially free of contaminating microorganisms or other macromolecules, e.g., polysaccharides, nucleic acids, or proteins. A purified composition comprising Bacillus coagulans bacteria or Bacillus coagulans bacteria extracellular product contains at least 75%, 85%, 95%, or 100% of the desired composition and is substantially free of other sub-cellular components such as cytoplasmic organelles. A purified composition comprising Bacillus coagulans bacteria is at least 60% the desired strain relative to the total population of cells. Preferably, the composition comprising Bacillus coagulans bacteria is at least 75%, more preferably at least 90%, and most preferably at least 99%, the desired strain relative to the total population of cells. For example, a purified population of bacteria is one that is at least 90%, 91%, 92%, 93%, 94%, 95%, 96%, 97%, 98%, 99%, or 100% the desired strain relative to the total population of cells.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to reduce visible signs of aging. Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is an illustration of a typical protein gel electrophoresis method.

FIG. 2 is a photograph depicting the results of a gel electrophoresis experiment with Bacillus coagulans supernatant (GBI-30/GB-30/Ganeden BC³⁰™/BC³⁰, ATCC Designation Number PTA-6086 metabolites) and cell wall fractions.

FIG. 3 is a photograph depicting the results of a gel electrophoresis experiment with Bacillus coagulans supernatant and cell wall fractions, wherein each fraction was further size-fractionated as follows: <3 kDa, 3-30 kDa, and 30-200 kDa.

FIG. 4 is a schematic representation of how PMN migration begins in the blood stream and moves into the tissue via transwell migration plates.

FIG. 5 is a line graph showing the effect of various fractions of Bacillus coagulans supernatant (MET) on leukotriene B4 (LTB4)-directed migration.

FIG. 6 is a line graph showing the effect of various fractions of Bacillus coagulans cell wall fractions (CW) on leukotriene B4 (LTB4)-directed migration.

FIG. 7 is a line graph demonstrating the effect of drying and rehydration of Bacillus coagulans supernatant (MET) and cell wall fractions (CW) on leukotriene B4 (LTB4)-directed migration.

FIG. 8 is a line graph illustrating the effect of various fractions of Bacillus coagulans supernatant (MET) on the expression of CD69 on NK cells.

FIG. 9 is a line graph showing the effect of various fractions of Bacillus coagulans cell wall fractions (CW) on the expression of CD69 on NK cells.

FIG. 10 is a line graph demonstrating the effect of drying and rehydration of Bacillus coagulans supernatant (MET) and cell wall fractions (CW) on the expression of CD69 on NK cells.

FIG. 11 is a line graph showing the effect of various fractions of Bacillus coagulans supernatant (MET) on lymphocyte proliferation.

FIG. 12 is a line graph illustrating the effect of various fractions of Bacillus coagulans cell wall fractions (CW) on lymphocyte proliferation.

FIG. 13 is a line graph demonstrating the effect of drying and rehydration of Bacillus coagulans supernatant (MET) and cell wall fractions (CW) on lymphocyte proliferation.

FIG. 14 is a line graph showing the effect of various fractions of Bacillus coagulans supernatant (MET) on the production of interleukin-2 (IL-2) by peripheral blood mononuclear cells (PBMCs).

FIG. 15 is a line graph illustrating the effect of various fractions of Bacillus coagulans cell wall fractions (CW) on the production of IL-2 by PBMCs.

FIG. 16 is a line graph demonstrating the effect of drying and rehydration of Bacillus coagulans supernatant (MET) and cell wall fractions (CW) on the production of IL-2 by PBMCs.

FIG. 17 is a line graph showing the effect of various fractions of Bacillus coagulans supernatant (MET) on the production of IL-4 by PBMCs.

FIG. 18 is a line graph illustrating the effect of various fractions of Bacillus coagulans cell wall fractions (CW) on the production of IL-4 by PBMCs.

FIG. 19 is a line graph demonstrating the effect of drying and rehydration of Bacillus coagulans supernatant (MET) and cell wall fractions (CW) on the production of IL-4 by PBMCs.

FIG. 20 is a line graph showing the effect of various fractions of Bacillus coagulans supernatant (MET) on the production of IL-6 by PBMCs.

FIG. 21 is a line graph illustrating the effect of various fractions of Bacillus coagulans cell wall fractions (CW) on the production of IL-6 by PBMCs.

FIG. 22 is a line graph demonstrating the effect of drying and rehydration of Bacillus coagulans supernatant (MET) and cell wall fractions (CW) on the production of IL-6 by PBMCs.

FIG. 23 is a line graph showing the effect of various fractions of Bacillus coagulans supernatant (MET) on the production of IL-10 by PBMCs.

FIG. 24 is a line graph illustrating the effect of various fractions of Bacillus coagulans cell wall fractions (CW) on the production of IL-10 by PBMCs.

FIG. 25 is a line graph demonstrating the effect of drying and rehydration of Bacillus coagulans supernatant (MET) and cell wall fractions (CW) on the production of IL-10 by PBMCs.

FIG. 26 is a line graph showing the effect of various fractions of Bacillus coagulans supernatant (MET) on the production of interferon gamma (IFN-γ) by PBMCs.

FIG. 27 is a line graph illustrating the effect of various fractions of Bacillus coagulans cell wall fractions (CW) on the production of IFN-γ by PBMCs.

FIG. 28 is a line graph demonstrating the effect of drying and rehydration of Bacillus coagulans supernatant (MET) and cell wall fractions (CW) on the production of IFN-γ by PBMCs.

FIG. 29 is a line graph showing the effect of various fractions of Bacillus coagulans supernatant (MET) on the production of tumor necrosis factor alpha (TNF-α) by PBMCs.

FIG. 30 is a line graph illustrating the effect of various fractions of Bacillus coagulans cell wall fractions (CW) on the production of TNF-α by PBMCs.

FIG. 31 is a line graph demonstrating the effect of drying and rehydration of Bacillus coagulans supernatant (MET) and cell wall fractions (CW) on the production of TNF-α by PBMCs.

FIG. 32 is a line graph showing the effect of Bacillus coagulans supernatant (MET) and cell wall fractions (CW) on the percentage of PBMCs that express CD14.

FIG. 33 is a line graph illustrating the effect of Bacillus coagulans supernatant (MET) and cell wall fractions (CW) on the expression of CD14 on CD14+ monocytes.

FIG. 34 is a line graph demonstrating the effect of Bacillus coagulans supernatant (MET) and cell wall fractions (CW) on the expression of CD80 on CD14+ monocytes.

FIG. 35 is a line graph showing the effect of Bacillus coagulans supernatant (MET) and cell wall fractions (CW) on the expression of CD86 on CD14+ monocytes.

FIG. 36 is a bar chart showing Novameter readings that demonstrated that the test product M-7293 (i.e., cream with Bonicel (Bacillus coagulans supernatant) dramatically increased the skin moisture content.

FIG. 37 is a bar chart showing Novameter readings that demonstrated that the test product M-7294 (i.e., cream without Bonicel (Bacillus coagulans supernatant) did not increase the skin moisture content.

FIG. 38 is a bar chart showing that the anti-aging test material (AMA Lab No.: M-7293 (Cream with Bonicel (Bacillus coagulans supernatant), Lot 28378) demonstrated a dramatic decrease compared to placebo treatment (AMA Lab No.: M-7294 (Cream without Bonicel, Lot 28378) in the Visioscan parameters of surface roughness (SEr) associated with the depth of fine and course wrinkles.

FIG. 39 is a bar chart showing Visioscan readings that demonstrated that the test product M-7294 (i.e., cream without Bonicel (Bacillus coagulans supernatant) did not decrease surface roughness associated with the depth of fine and course wrinkles.

FIG. 40 is a bar chart showing Cutometer measurements of the skin's Elasticity/Flexibility in the group treated with the test product M-7293 (i.e., cream with Bonicel (Bacillus coagulans supernatant)).

FIG. 41 is a bar chart showing Cutometer measurements of the skin's Elasticity/Flexibility in the group treated with the test product M-7294 (i.e., cream without Bonicel (Bacillus coagulans supernatant)).

FIG. 42 is a dot plot showing the results of a reverse photo engineering experiment to analyze wrinkle reduction in the presence of the test product M-7293 (i.e., cream with Bonicel (Bacillus coagulans supernatant)).

FIG. 43 is a dot plot showing the results of a reverse photo engineering experiment to analyze wrinkle reduction in the presence of the test product M-7294 (i.e., cream without Bonicel (Bacillus coagulans supernatant)).

FIG. 44 is a schematic showing test sites on a face.

### DETAILED DESCRIPTION OF THE INVENTION

The Bacillus coagulans bacterium described herein (e.g., ATCC Numbers: GBI-20 (GB-20), ATCC Designation Number PTA-6085; GBI-30 (GB-30/Ganeden BC³⁰™/BC³⁰/BC30), ATCC Designation Number PTA-6086; and GBI-40 (GB-40), ATCC Designation Number PTA-6087; see, U.S. Pat. No. 6,849,256 to Farmer), along with the extracellular products (i.e., metabolites/supernatants) thereof are useful in topical cosmetic formulations. The topical cosmetic formulations contain a supernatant obtained from the culture of B. coagulans, e.g., BC30. The supernatant is the metabolic byproduct produced during bacterial fermentation. It is naturally derived from Ganeden BC30, an organism that is generally regarded as safe (GRAS). The supernatant includes the following compounds: naturally derived L+ lactic acid, bacteriocin, hydrogen peroxide, enzymes, and other metabolites.

The cosmetic formulation described herein reduces inflammation, improves skin elasticity, improves skin hydration, reduces the appearances of fine lines and wrinkles, reduces under eye puffiness, reduces under eye dark circles, decreases skin pore size, reduces skin roughness, reduces skin redness, and/or improves skin flora, e.g., by reducing bacterial levels, reducing fungal levels, or reducing yeast levels. For example, the composition optionally inhibits the growth of pathogenic bacteria, fungus, or yeast. Delivery of the composition for skin care is accomplished using the supernatant or vegetative cells formulated into lotions, creams, gels, powders, scrubs, masks, shampoos, or conditioners.

The Effect of Bacillus coagulans Bacterium and Extracellular Product on Inflammation

The health benefits of the extra-cellular materials produced by Bacillus coagulans bacteria during their respective fermentation processes are described herein. The extra-cellular material called “supernatant” contains enzymes, lactic acid, hydrogen peroxide, bacteriocins, and other materials that are beneficial to a host.

The benefits of the supernatant from lactic acid bacteria on localized and systemic immune function are described in detail below. The compounds present in the supernatant from these bacteria have a profound effect on immune function as it pertains to accelerated healing and disease mitigation. These compounds include peptidoglycans, Lipotechoic acids and other organic molecules, which have a significant effect on inflammation and other host-cell interactions.

Inflammation is part of the complex biological response of skin and vascular tissues to harmful stimuli, such as pathogens, damaged cells, allergens and antigens. Inflammation is a protective attempt by the body to remove deleterious stimuli and to initiate the healing process through cytokine expression. Inflammation is a stereotyped response, and is considered a mechanism of innate immunity, as compared to adaptive immunity, which is specific for each pathogen or allergen.

Inflammation is important to disease mitigation. Without it, infections would never heal. As a result, progressive destruction of the tissue would compromise the survival of the organism or body. However, continued inflammation at the site of a wound after antibiotics or other anti-infective compounds are utilized can have deleterious effects on the healing process by restricting circulation to the infected site and prolonging the painful symptoms that accompany the infection. For this reason, steroid preparations are commonly utilized with many anti-infective strategies. This is also true with autoimmune and allergenic induced inflammation.

Inflammation can be classified as either acute or chronic. Acute inflammation is the initial response of the body to harmful stimuli, and is achieved by the increased movement of plasma and leukocytes from the blood into the injured tissues. A cascade of biochemical events (cytokine activity) propagates the inflammatory response, involving the local vascular system, the immune system, and various cells within the injured tissue. Prolonged inflammation, known as chronic inflammation, leads to a progressive shift in the type of cells present at the site of inflammation, and is characterized by simultaneous destruction and healing of the tissue from the inflammatory process.

Most of the unpleasant symptoms of an infection are the result of inflammation. By reducing the inflammation, the symptoms of the infection are reduced as well. In the case of athlete's foot, which is caused by the fungal species Thichophyton, very unpleasant symptoms accompany the infection. These symptoms may include itching, redness, burning and the formation of painful cracks in the skin that can bleed and lead to secondary infections. By reducing the inflammation associated with this fungal infection, circulation to the infected site is increased which allows nutrients and immune cells to migrate to the site to enhance recovery and accelerate the healing process. This mechanism is the same for other infections as well.

Described in detail below is the liquid fermentation product or supernatant of Bacillus coagulans BC³⁰. The Bacillus coagulans supernatant is manufactured under strict current good manufacturing practices (cGMP) guidelines using the most modern fermentation equipment and infrastructure. In clinical trials, the Bacillus coagulans supernatant modulates systemic and localized immune function, and assists the body in making proper immune decisions. This modulation includes down-regulation of inflammatory cytokine expression through a number of host-cell interactions between bacterial cell wall components.

For example, the immune modulating activity of the Bacillus coagulans supernatant includes: increasing systemic lymphocyte proliferation, increasing the maturation rate of dendritic cells, increasing Natural Killer Cell (NK) activation, favorably modulating TNF-α and other cytokine expression, reducing C-reactive protein (Systemic Inflammation Score), and increasing CD4 cell ratios in HIV+ patients.

As described in detail below, the Bacillus coagulans supernatant/fermentation product (e.g., supernatant of Ganeden Biotech BC³⁰) is a safe and effective compound for reduction of inflammation associated with infections and allergic reactions. Decreased inflammation score directly translates to reductions in the associated symptoms of infections (e.g., burning, itching, pain, swelling, redness, heat, and accumulation of immune cells), allergic reactions, and topical auto-immune manifestations, reduced incidence of secondary infection, and faster/accelerated healing.

As described in detail below, the Bacillus coagulans extracellular product (i.e., supernatant/fermentation product) has favorable effects on skin that is damaged by the consequences of aging or general exposure by reducing pore size, redness, roughness, wrinkles and fine lines, increasing hydration, reducing puffiness, and increasing elasticity. For example, in the anti-aging studies described below, the Bacillus coagulans supernatant decreased fine lines and wrinkles by 50%, increased skin hydration by 16.20%, reduced under eye puffiness by 8.33%, and increased general skin elasticity by 10.97%. Bacillus coagulans supernatant also decreased skin pore size by 27%-58%, decreased skin roughness by 20%, and reduced skin redness by 62%.

The Bacillus coagulans supernatant/fermentation product is formulated into virtually any cosmetic product without losing its activity. Formulations include, inter alia, creams, lotions, gels, shampoos, and cream rinses.

### Lactic Acid-Producing Bacteria

The bacteria described herein are non-pathogenic, non-toxigenic, and retain viability during storage. Since probiotics do not generally permanently colonize the host, they need to be administered regularly for any health promoting properties to persist. A probiotic lactic acid-producing bacterium suitable for use in the methods and compositions of the invention produces acid and is non-pathogenic. Purified and/or isolated Bacillus coagulans or the extracellular product of Bacillus coagulans is particularly useful as a probiotic in the compositions described herein. By “purified” or “substantially purified” is meant a Bacillus coagulans bacterium or the extracellular product of a Bacillus coagulans bacterium that is substantially free of contaminating microorganisms or other macromolecules, e.g., polysaccharides, nucleic acids, or proteins.

Purified defines a degree of sterility that is safe for administration to a human subject, e.g., lacking infectious or toxic agents. Specifically, as used herein, an “isolated” or “purified” Bacillus coagulans or extracellular product is substantially free of other cellular material or culture medium. A purified composition comprising Bacillus coagulans bacteria or an extracellular product of a Bacillus coagulans bacterium contains at least 75%, 85%, 95%, or 100% of the desired composition and is substantially free of other sub-cellular components such as cytoplasmic organelles. A purified composition comprising Bacillus coagulans bacteria is at least 60% the desired strain relative to the total population of cells. Preferably, the composition comprising Bacillus coagulans bacteria is at least 75%, more preferably at least 90%, and most preferably at least 99%, the desired strain relative to the total population of cells. For example, a purified population of bacteria is one that is at least 90%, 91%, 92%, 93%, 94%, 95%, 96%, 97%, 98%, 99%, or 100% the desired strain relative to the total population of cells. Purity is measured by any appropriate standard method.

The compositions include a lactic acid-producing bacterium, such as a spore-forming Bacillus species, e.g., B. coagulans. Preferably, the spore-forming Bacillus species of the invention is B. coagulans Hammer or a species derived therefrom. There are many suitable bacteria identified as described herein, although the invention is not limited to currently known bacterial species insofar as the purposes and objectives of the bacteria is described. The property of acid production is important to the effectiveness of the probiotic lactic acid-producing bacteria of this invention.

Exemplary methods and compositions are described herein using Bacillus coagulans extracellular product or Bacillus coagulans itself as a cosmetic agent. Purified Bacillus coagulans extracellular product is particularly useful in the compositions described herein. B. coagulans is non-pathogenic and is generally regarded as safe (i.e., GRAS classification) by the U.S. Federal Drug Administration (FDA) and the U.S. Department of Agriculture (USDA), and by those skilled in the art.

Bacillus coagulans

Bacillus coagulans is a non-pathogenic gram positive spore-forming bacteria that produces L(+) lactic acid (dextrorotatory) in fermentation conditions. It has been isolated from natural sources, such as heat-treated soil samples inoculated into nutrient medium (Bergey's Manual off Systemic Bacteriology, Vol. 2, Sneath, P. H. A., et al., eds., Williams & Wilkins, Baltimore, Md., 1986). Purified B. coagulans strains have served as a source of enzymes including endonucleases (e.g., U.S. Pat. No. 5,200,336), amylase (U.S. Pat. No. 4,980,180), lactase (U.S. Pat. No. 4,323,651), and cyclo-malto-dextrin glucano-transferase (U.S. Pat. No. 5,102,800). B. coagulans has been used to produce lactic acid (U.S. Pat. No. 5,079,164). A strain of B. coagulans (referred to as L. sporogenes; Sakaguti & Nakayama (ATCC 31284)) has been combined with other lactic acid producing bacteria and B. natto to produce a fermented food product from steamed soybeans (U.S. Pat. No. 4,110,477).

Bacterial species include Bacillus coagulans, e.g., Bacillus coagulans hammer, preferably Bacillus coagulans hammer strain Accession No. ATCC 31284, or one or more strains derived from Bacillus coagulans hammer strain Accession No. ATCC 31284 (e.g., ATCC Numbers: GBI-20, ATCC Designation Number PTA-6085; GBI-30 (BC³⁰), ATCC Designation Number PTA-6086; and GBI-40, ATCC Designation Number PTA-6087; see U.S. Pat. No. 6,849,256 to Farmer).

Bacillus coagulans was previously mis-characterized as a Lactobacillus and labeled as Lactobacillus sporogenes (Nakamura et al. 1988. Int. J. Syst. Bacteriol. 38: 63-73). However, initial classification was incorrect because Bacillus coagulans produces spores and excretes L(+)-lactic acid through metabolism. Both of these characteristics provide key features to the utility of Bacillus coagulans. These developmental and metabolic aspects required that the bacterium be classified as a lactic acid Bacillus.

In one aspect, a Bacillus coagulans strain is included in the composition in the form of vegetative cells. In another aspect, the Bacillus coagulans strain is included in the composition in the form of spores. Preferably, the Bacillus coagulans extracellular product or supernatant is utilized as a cosmetic agent in the compositions described herein. The invention also provides for including the Bacillus coagulans strain in the composition in the form of a powder, a dried cell mass, a stabilized paste, or a stabilized gel.

Because Bacillus spores are heat and pressure-resistant and can be stored as a dry powder, they are particularly useful for formulation into and manufacture of cosmetic compositions. Specifically, the probiotic organisms described herein, e.g., Bacillus coagulans strain GBI-30 or BC³⁰, ATCC Designation Number PTA-6086, can withstand the manufacturing process of cosmetic products. A Bacillus species is well suited for the present invention, particularly species having the ability to form spores which are relatively resistant to heat and other conditions, making them ideal for storage (shelf-life) in product formulations. Due to the shelf-stable properties of the Bacillus coagulans strains described herein, e.g., Bacillus coagulans strain GBI-30 or BC³⁰, ATCC Designation Number PTA-6086, the product formulations of the invention are not confined to a refrigerator and may be stored at room temperature. The Bacillus coagulans of the invention survives storage (shelf-life) from about 12 days to about 2 years; from about 1 month to about 18 months; from about 3 months to about 1 year; or from about 6 months to about 9 months.

The invention is directed to the surprising discovery that the extracellular products of lactic acid-producing bacteria, particularly Bacillus species, reduce the visible signs of aging. Specifically, the probiotic organisms described herein, e.g., Bacillus coagulans strain GBI-30 or BC³⁰, ATCC Designation Number PTA-6086, improve skin hydration/moisturization, improve skin elasticity, reduce the appearance of fine lines and wrinkles around the eye area, decrease the appearance of under-eye puffiness and dark circles, reduce the appearance of skin inflammation, reduce skin pore size, reduce skin roughness, and decrease skin redness.

The Bacillus coagulans extracellular product or Bacillus coagulans itself is topically administered. Any of a variety of methods for providing a bacterial composition can be used. In one aspect, a “spray-dry” method is used, in which the compositions are exposed in a low humidity chamber to an atomized mix containing a liquid composition, where the chamber is subsequently exposed to approximately 80-110° F. to dry the liquid, thereby impregnating a material of the composition with the components. In some cases, Bacillus coagulans bacteria in the form of a spray-dried powder is included in or on the surface of the compositions described herein.

The active ingredients (i.e., live bacteria or extracellular components), comprise between about 0.01% to about 10%; 0.01% to about 1%; or about 0.05% to about 0.1% by weight of the probiotic composition. Optionally, the isolated Bacillus coagulans comprise about 1 mg to about 10 mg; about 10 mg to about 1 g; or about 25 mg to about 75 mg by weight of the cosmetic composition.

### Micro-Encapsulation

In one aspect, the extracellular products of the lactic-acid producing bacteria or the lactic-acid producing bacteria themselves are incorporated into a microcapsule coating, using any micro-encapsulation process well-known in the art. The Bacillus coagulans or Bacillus coagulans extracellular product are packaged, or encapsulated, within another material in order to protect the bacteria from the surrounding environment. The capsules of the invention range in size from one-thousandth of a millimeter to seven millimeters.

The internal ingredients of the microcapsule are released from their shells in various ways, including mechanical rupture of the capsule wall, dissolution of the wall, melting of the wall and diffusion through the wall. Thus, micro-encapsulation provides additional protection to the isolated Bacillus bacterium or the extracellular product of the Bacillus bacterium during manufacturing and storage of the compositions of the invention. Physical methods of micro-encapsulation include pan coating, air-suspension coating, centrifugal extrusion, vibrational nozzle, and spray-drying. Chemical methods of micro-encapsulation include interfacial polymerization, in-situ polymerization, and matrix polymerization.

### Cosmetic Compositions

The invention is directed to the surprising discovery that the extracellular product of lactic acid-producing bacteria, particularly Bacillus species, reduces visible signs of aging. For example, the extracellular product of Bacillus coagulans improves skin hydration/moisturization, improves skin elasticity, reduces the appearance of fine lines and wrinkles around the eye area, and decreases the appearance of under-eye puffiness and dark circles. As described herein, the compositions are formulated in many configurations because the bacterium is present as a vegetative cell or as a spore, or both, depending on the species and form of the probiotic organism. Preferably, the extracellular product of the bacterium is utilized in the cosmetic compositions described herein.

Cosmetics are substances used to enhance the appearance or odor of the human body. Cosmetics include skin-care creams, lotions, powders, perfumes, lipsticks, eye and facial makeup, gels, deodorants, hand sanitizer, bath oils, bath salts, butters, and many other types of products. A subset of cosmetics is called “make-up,” which refers primarily to colored products intended to alter the user's appearance. The U.S. Food and Drug Administration (FDA) which regulates cosmetics in the United States defines cosmetics as: “intended to be applied to the human body for cleansing, beautifying, promoting attractiveness, or altering the appearance without affecting the body's structure or functions.”

Accordingly, the cosmetic compositions described herein include various skin care products. These include creams and lotions to moisturize the face and body which are typically formulated for different skin types, and treatment products to repair or hide skin imperfections (acne, wrinkles, dark circles under eyes, etc.). For each skin type, the correct types of products must be used in order to maintain healthy and attractive skin. Regular use of a suitable moisturizer benefits the skin, as it hydrates and prevents the dehydration of skin. Thus, the compositions described herein protect the skin against the drying influences of the environment, including the harsh effects of the sun, cold and heat. Oil free moisturizers are utilized for oily skins. Types of moisturizers include oil-in water emulsions and water-in-oil emulsions. For normal and combination skin, a water based moisturizer containing minimal oil is suitable. Sensitive and dry types of skin require moisturizers containing a high content of oil.

The cosmetic compositions described herein include natural or organic ingredients. All natural products contain mineral and plant ingredients, while organic products are made with organic agricultural products.

### Bacillus Coagulans Extracellular Product to Reduce Visible Signs of Aging

The compositions of the invention reduce visible signs of aging. Specifically, Bacillus coagulans extracellular products and Bacillus coagulans itself, e.g., Bacillus coagulans strain GBI-30 or BC³⁰, ATCC Designation Number PTA-6086, improve skin hydration/moisturization, improve skin elasticity, reduce the appearance of fine lines and wrinkles around the eye area, and decrease the appearance of under-eye puffiness and dark circles.

Accordingly, compositions comprising Bacillus coagulans bacteria extracellular product and Bacillus coagulans itself are administered to reduce visible signs of aging. For example, the bacteria extracellular product is administered in an amount that reduces visible signs of aging in the subject compared to the signs of aging in the subject prior to the administration. In some cases, a subject comprising a visible sign of aging is identified prior to administration of the bacteria. Preferably, the bacteria extracellular product is purified.

Skin hydration is increased by at least 1% following the administration of Bacillus coagulans bacteria extracellular product, e.g., skin hydration is increased by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, at least 95%, or at least 99% following the administration of Bacillus coagulans bacteria extracellular product compared to the skin hydration in the subject prior to the administration of Bacillus coagulans bacteria extracellular product.

Skin elasticity is increased by at least 1% following the administration of Bacillus coagulans bacteria extracellular product, e.g., skin elasticity is increased by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, at least 95%, or at least 99% following the administration of Bacillus coagulans bacteria extracellular product compared to the skin elasticity in the subject prior to the administration of Bacillus coagulans bacteria extracellular product.

Fine lines and wrinkles are reduced by at least 1% following the administration of Bacillus coagulans bacteria extracellular product, e.g., fine lines and wrinkles are reduced by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, at least 95%, or at least 99% following the administration of Bacillus coagulans bacteria extracellular product compared to the quantity of fines lines and wrinkles in the subject prior to the administration of Bacillus coagulans bacteria extracellular product.

Under eye puffiness and/or under eye dark circles are reduced by at least 1% following the administration of Bacillus coagulans bacteria extracellular product, e.g., under eye puffiness and/or under eye dark circles are reduced by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, at least 95%, or at least 99% following the administration of Bacillus coagulans bacteria extracellular product compared to the under eye puffiness and/or under eye dark circles in the subject prior to the administration of Bacillus coagulans bacteria extracellular product.

The compositions of the invention comprise a skin aging-reducing amount of Bacillus coagulans bacteria extracellular product. For example, the Bacillus coagulans bacteria extracellular product is provided at a concentration of 1%, 2%, 3%, 4%, 5%, 6%, 7%, 8%, 9%, 10%, 15%, 20%, 25%, 35%, 50%, 60%, 75%, 90%, 99% or 100% in the cosmetic compositions described herein.

Exemplary formulations of the compositions of the invention include a suspension, a powder, a cream, a lotion, a salve, a gel, a scrub, a mask, a shampoo, and a conditioner.

The compositions of the invention are administered topically. The compositions are administered at least once per day, e.g., at least twice per day, at least 3 times per day, at least 4 times per day, or at least 5 times per day. Preferably, the compositions are administered for at least 24 hours, at least 48 hours, at least 72 hours, or for at least 7 days, at least 14 days, at least 30 days, at least 60 days, at least 90 days, or for at least 4 months, at least 6 months, at least 9 months, or for at least 1 year, at least 2 years, or at least 3 years.

### Example 1

### Preparation of Bacillus coagulans Cultures

Bacillus coagulans Hammer bacteria (ATCC Accession No. 31284) was inoculated and grown to a cell density of about 10⁸ to 10⁹ cells/ml in nutrient broth containing 5 g Peptone, 3 g Meat extract, 10-30 mg MnSO₄, and 1,000 ml distilled water, adjusted to pH 7.0, using a standard airlift fermentation vessel at 30° C. The range of MnSO₄ acceptable for sporulation is 1 mg/l to 1 g/l. The vegetative cells can actively reproduce up to 45° C., and the spores are stable up to 90° C. After fermentation, the B. coagulans bacterial cells or spores are collected using standard methods (e.g., filtration, centrifugation) and the collected cells and spores can be lyophilized, spray-dried, air-dried, or frozen. The supernatant from the cell culture is collected and used as an extracellular agent secreted by B. coagulans.

A typical yield from the above culture is in the range of about 10⁹ to 10¹⁰ viable spores and more typically about 100 to 150 billion cells/spores per gram before drying. Spores maintain at least 90% viability after drying when stored at room temperature for up to ten years, and thus the effective shelf life of a composition containing B. coagulans Hammer spores at room temperature is about 10 years.

### Example 2

### Preparation of Bacillus coagulans Spores

A culture of dried B. coagulans spores was prepared as follows. Ten million spores were inoculated into a one liter culture containing 24 g potato dextrose broth, 10 g of enzymic-digest of poultry and fish tissue, 5 g of FOS and 10 g MnSO₄. The culture was maintained for 72 hours under a high oxygen environment at 37° C. to produce culture having about 150 billion cells per gram of culture. Thereafter, the culture was filtered to remove culture medium liquid, and the bacterial pellet was resuspended in water and freeze-dried. The freeze-dried powder is then ground to a fine powder using standard good manufacturing practice (GMP).

### Example 3

### Production of Bacillus coagulans Supernatant

Bacillus coagulans GBI-30 (GB-30/Ganeden BC³⁰™/BC³⁰), ATCC Designation Number PTA-6086, supernatant is produced as outlined below.

TABLE 1
Trace Mineral solution (pH will be ~3.0 to 3.5).
Add one milliliter Trace mineral stock per 1 liter of medium.

| Ingredient | For GYE Media (gm/L) | |
|---|---|---|
| NaCl | 10 | gm |
| FeSO₄•7 H₂O | 18 | gm |
| MnSO₄•5 H₂O | 16 | gm |
| ZnSO₄•7 H₂O | 1.6 | gm |
| CuSO₄•5 H₂O | 1.6 | gm |
| CoSO₄•7 H₂O | 1.6 | gm |
| DI Water | 1 | Liter |

Solution will be pink. Stable in refrigerator for ~60 days.

TABLE 2
Standard Media.

| Ingredient | For GYE Media (gm/L) | |
|---|---|---|
| Yeast Extract | 5 | gm |
| (Difco/Amberex) | | |
| KH₂PO₄ | 0.5 | gm |
| K₂HPO₄ | 0.5 | gm |
| MgSO₄ | 0.3 | gm |
| Trace Mineral Solution | 1 | ml. |
| Peptone | 5 | gm |
| Glucose | 5 gm (70% solution sterilized | |
| | separately; added to media | |
| | after autoclave) | |
| Water | 1 | Liter |

### Fermentation Settings

### Shake Flask Inoculum Prep

1. Aseptic transfer of 1.0 ml. Bacillus coagulans BC³⁰ working stock into each of 12 flasks with 1 liter medium.
2. Record initial pH, OD₆₀₀, and glucose concentration.

### 3. Shaker: 200 RPM

4. pH: should stay above 5.5
5. Incubate for 5 days at 37° C.
6. Alternate method: Add 5 gm glucose and 5 gm peptone per liter after day 1, 2, 3, and 4.
7. Bring to ambient and adjust pH to 4.0 (Phosphoric acid or NaOH).
8. Centrifuge to sediment cell mass and filter supernatant to pass a 0.2 μM pore size.
9. Final product: A. keep refrigerated in aseptic vessel; or B. lyophilization; or C. add approved preservative in specified concentration.
An alternative protocol is provided below:
Production Bacillus coagulans BC30
Trace Mineral solution (pH will be ˜3.0 to 3.5).

TABLE 3
Add one milliliter Trace mineral stock per 1 liter of medium.

| Ingredient | For GYE Media (gm/L) | | Fermentor Protocol (gm/L) | |
|---|---|---|---|---|
| NaCl | 10 | gm | 10 | gm |
| FeSO₄•7 H₂O | 18 | gm | 20 | gm |
| MnSO₄•5 H₂O | 16 | gm | — | |
| MnSO₄•H₂O | — | | 20 | gm |
| ZnSO₄•7 H₂O | 1.6 | gm | 5 | gm |
| CuSO₄•5 H₂O | 1.6 | gm | 5 | gm |
| CoSO₄•7 H₂O | 1.6 | gm | 5 | gm |
| DI Water | 1 | Liter | 1 | Liter |

Solution will be pink. Stable in refrigerator for ~60 days.

TABLE 4
Standard Media.

| | For GYE Media | | Fermentor Protocol | |
|---|---|---|---|---|
| Ingredient | (gm/L) | | (gm/L) | |
| Yeast Extract | 5 | gm | 5 | gm |
| (Difco/Amberex) | | | | |
| KH₂PO₄ | 0.5 | gm | 1 | gm |
| K₂HPO₄ | 0.5 | gm | 1 | gm |
| MgSO₄ | 0.3 | gm | — | |
| MgSO₄•7 H₂O | — | | 1 | gm |
| Trace Mineral Solu- | 1 | ml. | 1 | ml. |
| tion | | | | |
| Peptone | 5 | gm | 2 | gm |
| Glucose | 5 gm (added before | | 5 gm (70% solution sterilized | |
| | autoclave) | | separately; added to media | |
| | | | after autoclave) | |
| Water | 1 | Liter | 1 | Liter |

| | | | |
|---|---|---|---|
| antifoam | — | 0.25 | ml. |

### Fermentation Settings

### Shake Flask Inoculum Prep

1. Aseptic transfer of 1.0 ml. Bacillus coagulans BC³⁰ working stock into each of 12 flasks with 1 liter medium.
2. Record initial pH, OD₆₀₀, and glucose concentration.

### 3. Temp: 45° C.

### 4. Shaker: 300 RPM

5. pH: should stay above 5.5
6. At OD₆₀₀=1.5 to 2.0, pH will be ˜5.5
7. Time: 10 hr to 18 hrs to develop target OD₆₀₀=1.5 to 2.0

### Secondary Seed Fermentation (3000 L)

1. Sterilize 60 minutes at 121° C.
2. Add sterile glucose solution (70%) to 5 gm/L final conc.

### 3. Temp: 45° C.

4. pH: 6.4
5. Agitation: 60 max-RPM

### 6. DO: 20% to 30%

### 7. Pressure: 0.5 gm

8. On DO spike when initial glucose has been depleted, add 5 gm/L bolus but no more than conc. of 10 gm/L.
9. In 12 to 15 hours when OD₆₀₀=15 to 20, transfer to 90,000 L tank (should have less than 5% spores as free or intracellular).

### Final Fermentation (90,000 L)

1. Medium prep. and fermentor settings as above for 65,000 liters.
2. At DO spike (−4 hrs) start feeding 450 L/hr and increase feed to 800 to 1000 L/hr over a period of 5 hours keeping glucose conc. at 5 to 10 gm/L.
3. At 12 to 15 hours at (OD₆₀₀=20 to 50 (use DI water blank at OD>5) dry wt= 6/12 gm/L) stop glucose feed and continue fermentation until glucose is depleted.
4. Chill and adjust to pH 4.0 (85% phosphoric acid or conc. NaOH).
5. Centrifuge to sediment cells and spores

In some cases, medium is generated using dried Corn Steep Liquor solids, supplemented with yeast extract, and either Soy flour or Cottonseed flour added for protein/nitrogen with additional glucose, as needed.

An exemplary formulation comprising Bacillus coagulans extracellular product includes the following ingredients: Bacillus coagulans extracellular product, water, isopropyl myristate, isocetyl stearate, glycerin, ricinus communis (castor) seed oil, hydrogenated vegetable oil, vegetable oil, hydrogenated castor oil, cetyl alcohol, polyacrylamide, c13-14 isoparaffin, laureth-7, ethylhexyl methoxycinnamate, squalene, laneth-16, ceteth-16, oleth-16, steareth-16, caprylyl glycol, phenoxyethanol, hexylene glycol, and fragrance.

### Example 4

### Effect of Bacillus coagulans Extracellular Product on Aging

A human clinical trial using the supernatant-containing formulation was conducted using 24 female subjects, ages 35-60. Skin condition at baseline, after using active formula, and after using placebo was evaluated.

At 4 weeks, the cream plus supernatant increased skin hydration by 7.13% more than a placebo cream, while the cream plus supernatant increased skin elasticity by 3.11% more than a placebo cream. Off the silicone replicas, the cream plus supernatant decreased the number of coarse skin lines by 20.57% more than a placebo cream, while the cream plus supernatant increased skin smoothness by 4.33% more than a placebo cream. The cream plus supernatant decreased skin shadows by 7.09% more than a placebo cream. Off of visual evaluation, the cream plus supernatant had 17% increase in the number of subjects showing improvement of eye area fine lines and wrinkles more than a placebo cream, while the cream plus supernatant had 8.33% increase in the number of subjects showing improvement of under eye puffiness more than a placebo cream.

The results are summarized below.

| Indication | Improvement over baseline |
|---|---|
| Skin hydration | 16.20% |
| Skin elasticity | 10.97% |
| Reduction in fine lines and wrinkles | 50.00% |
| Under eye puffiness | 8.33% |

Described below is a summary of results of a double blind study to compare the efficacy of an anti-aging skin care product versus a placebo. The test products references in this example are “product A” and “product B.” Product A is the placebo cream, while product B is cream with 5% of the extracellular product/supernatant of Bacillus coagulans GBI-30 (GB-30/Ganeden BC³⁰™/BC³⁰/BC30), ATCC Designation Number PTA-6086.

The Bacillus coagulans was cultured in RPMI 1640. As described herein, RPMI 1640 with and without glutamate is an acceptable culture medium for the production of Bacillus coagulans supernatant. See, e.g., Jensen et al., 2010 BMC Immunology, 11:15, incorporated herein by reference. In some cases, medium is supplemented with serum, e.g., fetal calf serum. In other cases, the medium is serum free.

### Skin Hydration

Twenty-four female subjects (twelve in each treatment group), ranging in age from 35-60 years, consented, enrolled and completed the clinical study to assess the efficacy of two test products: Product A (placebo cream) and Product B (cream with 5% extracellular product of Bacillus coagulans).

TABLE 5

| | Percent Change In Mean Skin | |
|---|---|---|
| | Hydration From Baseline | |
| | Product A | Product B |
| Interval | (N = 12) | (N = 12) |
| 4 Weeks Post-Treatment | 9.07%* (p ≦ 0.05) | 16.20%* (p ≦ 0.001) |
| 8 Weeks Post-Treatment | 13.13%* (p ≦ 0.001) | 16.38%* (p ≦ 0.001) |

*Statistically significant value

There was a statistically significant increase in skin capacitance after 4 weeks and 8 weeks of product application for Product A, compared to baseline values. This indicates an increase in skin hydration. Furthermore, there was a statistically significant increase in skin capacitance after 4 weeks and 8 weeks of product application for Product B when compared against baseline values.

### 4 Weeks

Test Product A
83.33% of subjects showed an improvement in skin hydration.

Test Product B
91.67% of subjects showed an improvement in skin hydration.

### 8 Weeks

Test Product A
83.33% of subjects showed an improvement in skin hydration.

Test Product B
91.67% of subjects showed an improvement in skin hydration.

TABLE 6
Differences Between Product A and Product B.

| Interval | Difference between Product A and Product B |
|---|---|
| (N = 24) | (Product A − Product B) |
| 4 Weeks Post-Treatment | −7.13% |
| 8 Weeks Post-Treatment | −3.24% |

Positive differences indicate that Product A was more hydrating

There were no statistically significant differences from baseline in skin hydration between the two (2) test products at Week 4 or Week 8 post-treatment.

TABLE 7
Differences Between Product B and Product A.

| Interval | Difference between Product A and Product B |
|---|---|
| (N = 24) | (Product B − Product A) |
| 4 Weeks Post-Treatment | 7.13% |
| 8 Weeks Post-Treatment | 3.24% |

Positive differences indicate that Product B was more hydrating

There were no statistically significant differences from baseline in skin hydration between the two (2) test products at Week 4 or Week 8 post-treatment.

TABLE 8
Skin Elasticity.

Percent Change In Mean Skin
Elasticity From Baseline

| | Product A | Product B |
|---|---|---|
| Interval | (N = 14) | (N = 14) |
| 4 Weeks Post-Treatment | 7.86% | 10.97% |
| 8 Weeks Post-Treatment | −2.27% | 2.86% |

Positive value indicates improvement in skin elasticity

There was an increase in skin elasticity after 4 weeks of product application for Product A when compared against baseline. Furthermore, there was an increase in skin elasticity after 4 weeks and 8 weeks of product application for Product B when compared against baseline values.

### 4 Weeks

Test Product A
66.67% of subjects showed an improvement in skin elasticity.

Test Product B
66.67% of subjects showed an improvement in skin elasticity.

### 8 Weeks

Test Product A
50.00% of subjects showed an improvement in skin elasticity.

Test Product B
75.00% of subjects showed an improvement in skin elasticity.

TABLE 9
Differences Between Product A and Product B.

| Interval | Difference between Product A and Product B |
|---|---|
| (N = 28) | (Product A − Product B) |
| 4 Weeks Post-Treatment | −3.11% |
| 8 Weeks Post-Treatment | −5.14% |

Positive differences indicate that Product A site more elastic
* Statistically significant value (p < 0.05)

There were no statistically significant differences from baseline in skin elasticity between the two (2) test products at Week 4 or Week 8 post-treatment.

TABLE 10
Differences Between Product B and Product A.

| Interval | Difference between Product A and Product B |
|---|---|
| (N = 28) | (Product B − Product A) |
| 4 Weeks Post-Treatment | 3.11% |
| 8 Weeks Post-Treatment | 5.14% |

Positive differences indicate that Product A site more elastic
* Statistically significant value (p < 0.05)

There were no statistically significant differences from baseline in skin elasticity between the two (2) test products at Week 4 or Week 8 post-treatment.

### Periocular Wrinkles and Fine Lines (Silicone Replicas)

Parameters for Skin Texture: Rz and Ra=skin roughness texture parameters; decreases in Rz and/or Ra indicate an increase in skin smoothness. IDL=length of line; decrease in IDL indicates an increase in skin smoothness. Shadows=area of shadows cast by all lines; decrease in Shadows indicates an increase in skin smoothness. NumWr=total number of shadowy features; decrease in NumWr indicates an increase in skin smoothness. Parameters for Number and Depth of Fine and Coarse Lines: FNum=number of markers indicative of coarse or fine lines per mm; decrease in FNum indicates a decrease in number of coarse or fine lines. Spacing=mean distance between adjacent strong shadow features; increase in Spacing indicates a decrease in number of coarse or fine lines. Breadth=depth of the wrinkle/line producing shadow; decrease in breadth indicates a decrease in depth of coarse or fine lines.

TABLE 11

| | Test Product | |
|---|---|---|
| | A | B |
| Coarse at Week 4 | | |
| % Change in Rz (negative value indicates | 6.92% | 5.57% |
| increase in skin smoothness) | (p = 0.065) | (p = 0.201) |
| % Change in Ra (negative value indicates | 4.25% | 4.86% |
| increase in skin smoothness) | (p = 0.107) | (p = 0.393) |
| % Change in IDL (negative value indicates | 14.61% | 18.83% |
| increase in skin smoothness) | (p ≦ 0.01) | (p = 0.003) |
| % Change in Shadows (negative value | 7.15% | −5.13% |
| indicates increase in skin smoothness) | (p = 0.581) | (p = 0.508) |
| % Change in NumWr (negative value | 24.45% | 29.66% |
| indicates increase in skin smoothness) | (p = 0.012) | (p = 0.004) |
| % Change in FNUM (negative value | −16.37% | −36.94% |
| indicates decrease in number of coarse lines) | (p ≦ 0.001) | (p = 0.003) |
| % Change in Spacing (positive value | 5.56% | −6.54% |
| indicates decrease in number of coarse lines) | (p = 0.517) | (p = 0.399) |
| % Change in Breadth (negative value | 10.31% | 1.04% |
| indicates decrease in depth of coarse lines) | (p = 0.061) | (p = 0.806 |
| Coarse at Week 8 | | |
| % Change in Rz (negative value indicates | 1.32% | 2.92% |
| increase in skin smoothness) | (p = 0.789) | (p = 0.63) |
| % Change in Ra (negative value indicates | 2.56% | −0.44% |
| increase in skin smoothness) | (p = 0.714) | (p = 0.413) |
| % Change in IDL (negative value indicates | 4.07% | 5.76% |
| increase in skin smoothness) | (p = 0.528) | (p = 0.429) |
| % Change in Shadows (negative value | −11.56% | −7.41% |
| indicates increase in skin smoothness) | (p = 0.317) | (p = 0.233) |
| % Change in NumWr (negative value | 0.88% | 15.85% |
| indicates increase in skin smoothness) | (p = 0.935) | (p = 0.15) |
| % Change in FNUM (negative value | −16.32% | −38.43% |
| indicates decrease in number of fine lines) | (p ≦ 0.001) | (p ≦ 0.001) |
| % Change in Spacing (positive value | 16.23% | 20.82% |
| indicates decrease in number of fine lines) | (p = 0.129) | (p = 0.185) |
| % Change in Breadth (negative value | 13.75% | 13.18% |
| indicates decrease in depth of fine lines) | (p = 0.092) | (p = 0.064) |
| Fine Lines at Week 4 | | |
| % Change in Rz (negative value indicates | 1.40% | −2.93% |
| increase in skin smoothness) | (p = 0.713) | (p = 0.527) |
| % Change in Ra (negative value indicates | 0.05% | −8.22% |
| increase in skin smoothness) | (p = 0.991) | (p = 0.066) |
| % Change in IDL (negative value indicates | 6.71% | 1.82% |
| increase in skin smoothness) | (p = 0.14) | (p = 0.774) |
| % Change in Shadows (negative value | −11.18% | −18.27% |
| indicates increase in skin smoothness) | (p = 0.347) | (p = 0.173) |
| % Change in NumWr (negative value | 12.15% | 9.49% |
| indicates increase in skin smoothness) | (p = 0.343) | (p = 0.527) |
| % Change in FNUM (negative value | −16.65% | −14.32% |
| indicates decrease in number of fine lines) | (p ≦ 0.001) | (p = 0.013) |
| % Change in Spacing (positive value | 26.98% | 8.38% |
| indicates decrease in number of fine lines) | (p = 0.097) | (p = 0.47) |
| % Change in Breadth (negative value | 6.92% | 2.53% |
| indicates decrease in depth of fine lines) | (p = 0.034) | (p = 0.521) |
| Fine Lines at Week 8 | | |
| % Change in Rz (negative value indicates | 10.60% | 6.15% |
| increase in skin smoothness) | (p = 0.076) | (p = 0.123) |
| % Change in Ra (negative value indicates | 12.14% | 6.01% |
| increase in skin smoothness) | (p = 0.088) | (p = 0.158) |
| % Change in IDL (negative value indicates | 16.29% | 15.96% |
| increase in skin smoothness) | (p = 0.046) | (p = 0.006) |
| % Change in Shadows (negative value | 17.82% | 21.85% |
| indicates increase in skin smoothness) | (p = 0.154) | (p = 0.301) |
| % Change in NumWr (negative value | 33.92% | 49.86% |
| indicates increase in skin smoothness) | (p = 0.005) | (p = 0.003) |
| % Change in FNUM (negative value | −20.19% | −11.61% |
| indicates decrease in number of fine lines) | (p = 0.006) | (p = 0.009) |
| % Change in Spacing (positive value | −5.45% | −18.43% |
| indicates decrease in number of fine lines) | (p = 0.709) | (p = 0.051) |
| % Change in Breadth (negative value | 14.06% | 11.40% |
| indicates decrease in depth of fine lines) | (p = 0.009) | (p = 0.009) |

### Visual Evaluations of Clinical Photographs

### Week 4

### Test Product A

No statistically significant overall improvement in appearance of eye area fine lines and wrinkles (N=12)

33.33% of subjects showed an improvement in appearance of eye area fine lines and wrinkles (N=12)

No statistically significant overall improvement in appearance of under eye puffiness. (N=8)

0.00% of subjects showed an improvement in appearance of under eye puffiness. (N=8)

No statistically significant overall improvement in appearance of dark circles. (N=9)

22.22% of subjects showed an improvement in appearance of dark circles. (N=9)

### Test Product B

No statistically significant overall improvement in appearance of eye area fine lines and wrinkles (N=12)

50.00% of subjects showed an improvement in appearance of eye area fine lines and wrinkles (N=12)

No statistically significant overall improvement in appearance of under eye puffiness. (N=12)

8.33% of subjects showed an improvement in appearance of under eye puffiness. (N=12)

No statistically significant overall improvement in appearance of dark circles. (N=11)

9.09% of subjects showed an improvement in appearance of dark circles. (N=11)

### Week 8

### Test Product A

No statistically significant overall improvement in appearance of eye area fine lines and wrinkles (N=12)

25.00% of subjects showed an improvement in appearance of eye area fine lines and wrinkles (N=12)

No statistically significant overall improvement in appearance of under eye puffiness. (N=8)

37.50% of subjects showed an improvement in appearance of under eye puffiness. (N=8)

No statistically significant overall improvement in appearance of dark circles. (N=9)

44.44% of subjects showed an improvement in appearance of dark circles. (N=9)

### Test Product B

No statistically significant overall improvement in appearance of eye area fine lines and wrinkles (N=12)

41.67% of subjects showed an improvement in appearance of eye area fine lines and wrinkles (N=12)

No statistically significant overall improvement in appearance of under eye puffiness. (N=12)

25.00% of subjects showed an improvement in appearance of under eye puffiness. (N=12)

No statistically significant overall improvement in appearance of dark circles. (N=11)

36.36% of subjects showed an improvement in appearance of dark circles. (N=11)

### Test Products Comparison (Product a Versus Product B)

There were no statistically significant differences from baseline in appearance of eye area fine lines and wrinkles between the two (2) test products at Week 4 or Week 8 post-treatment.

There were no statistically significant differences from baseline in appearance of under eye puffiness between the two (2) test products at Week 4 or Week 8 post treatment.

There were no statistically significant differences from baseline in appearance of dark circles between the two (2) test products at Week 4 or Week 8 post-treatment.

TABLE 12
Self-Assessment Post-Treatment Questionnaire.

| | Percent of Subjects “Agreeing” | |
|---|---|---|
| | Product A | Product B |
| Statement | (N = 12) | (N = 12) |
| The skin around my eyes feels more | 75.00% | 66.67% |
| hydrated/moisturized. | | |
| The fine lines/wrinkles around my | 66.67% | 58.33% |
| eyes are less visible. | | |
| The skin around my eyes feels more | 75.00% | 75.00% |
| toned/firmer. | | |
| The skin around my eyes is less puffy. | 83.33% | 66.67% |
| The dark circles under my eyes are | 75.00% | 58.33% |
| less visible. | | |
| The skin around my eyes feels | 91.67% | 83.33% |
| smoother. | | |

Bold Values indicate statically significant.

### CONCLUSION

As summarized above, Product B (cream plus 5% extracellular product of Bacillus coagulans) showed greater improvement than Product A (placebo) when compared to baseline for the following:

Significantly more hydrating (p<0.050) at Week 4 and Week 8 post-treatment.

Larger number of subjects showed improved hydration at Week 4 and Week 8 post-treatment.

Improved elasticity at Week 4 and Week 8 post-treatment.

Larger number of subjects showed improved elasticity at Week 8 post-treatment.

Improvement of fine lines and wrinkles at Week 4 and Week 8 post-treatment (Visual Grading)

Improvement of under eye puffiness at Week 4 post-treatment.

Decreased the number of coarse lines at Week 4 post-treatment (Silicone Replicas)

Increased skin smoothness of coarse lines at Week 8 post-treatment (Silicone Replicas)

Increased skin smoothness of fine lines at Week 4 post-treatment (Silicone Replicas)

Decreased the number of fine lines at Week 8 post-treatment (Silicone Replicas)

Product A showed a greater improvement than Product B when compared to baseline for the following:

Improvement in the appearance of under eye dark circles at Week 4 and Week 8 post-treatment.

Improvement of under eye puffiness at Week 8 post-treatment.

Decreased the number of fine lines at Week 8 post-treatment (Silicone Replicas)

The experimental details of the results summarized above are provided below.

### Study Objective

Evaluate and compare the effectiveness of a topical anti-aging product versus placebo to:

1) Improve skin hydration/moisturization
2) Improve skin elasticity
3) Reduce the appearance of fine lines and wrinkles around the eye area
4) Decrease the appearance of under-eye puffiness
5) Decrease the appearance of dark circles

### Study Participation Recruitment

Panel selection was accomplished by advertisements in local periodicals, community bulletin boards, phone solicitation, electronic media or any combination thereof.

### Inclusion Criteria

a. Female (any race)

b. 35-60 years of age

c. Individuals who were free of any dermatological or systemic disorder, which would interfere with the results, at the discretion of the Investigator.

d. Individuals who were in good general health.

e. Individuals who completed a preliminary medical history and photography release form.

f. Individuals who read, understood and signed an informed consent document.

g. Individuals who were able to and agreed to cooperate with the Investigator and research staff, apply the test product according to the study protocol, and complete the full course of the study.

h. Individuals who were not concurrently participating in any other clinical study and had not participated in any facial anti-aging study in the past 30 days.

i. Individuals who showed presence of mild/moderate/fine lines and wrinkles around the eye areas (crow's feet).

j. Individuals who showed presence of mild/moderate dark circles under the eyes (25% of population in each treatment group).

k. Individuals who showed presence of mild/moderate under eye puffiness (25% of population in each treatment group).

l. Individuals who agreed to refrain from excessive sun exposure which may result in facial sunburn, tanning or wind-burn during the study.

m. Individuals who agreed to refrain from using all face/cosmetic products (e.g., soaps, creams, lotions), with the exception of products provided by the testing facility and eye and lip products for the duration of the study.

n. Individuals who agreed not to wear facial make up (including lip and eye make up) on their study day visits.

### Exclusion Criteria

a. Individuals who had a history of any acute or chronic disease that would interfere with or increase the risk on study participation.

b. Individuals with an active (flaring) disease or chronic skin allergies (atopic dermatitis/eczema), or had recently treated skin cancer (within the last 12 months).

c. Individuals with damaged skin in close proximity to test sites (e.g., sunburn, tattoos, scars or other disfigurations).

d. Individuals who had any history, which, in the Investigator's opinion, indicates the potential for harm to the subject or places the validity of the study in jeopardy.

e. Individuals who indicated that they were pregnant, planning a pregnancy or nursing.

f. Individuals who used injectable insulin to control their diabetes.

g. Individuals who had any medical procedure, such as laser resurfacing, or plastic surgery to the test areas within the last 12 months. This included Botox, Restylyn, collagen or other cosmetic filling procedures.

h. Individuals who were currently using or during the last 3 months had used, Retin A, or other Rx/OTC Retinyl A, hydroquinone (skin lightening) or other astringent derived products or alpha hydroxyl acid treatments for photo-aging and fine lines/wrinkles

i. Individuals who had a known history of hypersensitivity to any cosmetics, personal care products and alpha hydroxy acid products.

### Experimental Techniques

### Bioinstrumental Method to Measure Moisture Content of Human Skin

The use of moisturizers affects the water content of the outermost layers of skin, i.e., the stratum corneum (SC) (Jemec G B, Serup J. Epidermal Hydration and Skin Mechanics Acta Derm. Venereal.: 70: 245-250 (1990)). Changes in skin conductance, impedance or capacitance are used to study epidermal hydration in vivo. The measurement is made on the difference in dielectric constant; skin has a low dielectric constant and water has a high dielectric constant of 81. When skin is hydrated, conductance and capacitance increases and impedance decreases. The measuring capacitor shows changes in capacitance according to the moisture content of the tissue. A glass lamina separates the metallic tracks in the probe head from the skin in order to prevent current conduction in the tissue. An electric scatter field penetrates the skin during the measurement and the dielectricity is determined

Corneometer CM 825 (Courage+Khazaka Electronic GmbH, Köln, Germany) was used to measure the electrical capacitance of the skin.

### Bioinstrumental Method to Measure Elasticity of Human Skin

The biomechanical properties of human skin are a complex combination of elastic (elastin fibers) and viscous (collagen fibers and surrounding intercellular ground substance) components. The Cutometer allows the measurement of the viscoelastic properties of the skin in vivo. See, Undine B, Elsner P. Hardware and Measuring Principle: The Cutometer. In the Bioengineering of the Skin—Skin Biomechanics 2002; Pp 91-98; and Agache P, Varchon D. Skin Mechanical Function. In the Measuring the Skin. 2004; Pp 429-467, each of which is incorporated herein by reference. The measuring principle of the Cutometer is based on suction. A defined negative air pressure is created and applied on the skin surface through the opening of a probe drawing the skin into its aperture. The resulting vertical deformation of the skin is measured by determining the depth of skin penetration into the probe. This is achieved by a noncontact optical system consisting of a light transmitter and a light recipient. Two glass prisms project the light from transmitter to recipient, where the diminution of the infrared light beam depending on the penetration depth of the skin is measured.

Cutometer MPA 580 (Courage+Khazaka Electronic GmbH, Köln, Germany) was used to measure skin elasticity.

### Measurement of Fine Lines and Wrinkles

This was achieved by obtaining a topical 3D micro-anatomical profilometry via silicon replicas. See, Skin Res. Technol., 2: 112-117, 2002. Generally, sites evaluated for fine lines and wrinkles include the periocular areas located on the side of the eyes (Crow's feet) Two (2)×two (2) cm adhesive templates were affixed to the test site and Replifo vinyl silicone (Cuderm Corporation, Dallas, Tex.) was dispensed onto the template demarcated areas. After ˜5 minutes, the replicas were cured and gently removed from the skin surface. Image analysis using a Cohu solid state B&W camera, 50 mm lens/30 mm extension, Coreco TCI Ultra frame grabber interfaced with an IBM compatible PC is conducted by Cuderm Corporation, Dallas, Tex.

Specifically, during the image analysis phase, a collimated light source was directed at a 25-degree angle from the place of the replica. The replica was gently placed in the holder and was rotated to align for normal or parallel exposure to the incident light direction. Further changes in the gradient of light intensity can produce changes in luminance, which in turn is used to assess changes in skin roughness displayed by the replica. The normal sampling orientation provides texture measurements sensitive to the major expression-induced lines and the parallel sampling orientation provided texture measurements sensitive to the minor fine lines.

The shadow texture produced by the oblique lighting of the negative replica is analyzed by two types of assay methods:

1) Measuring the luminance along a set of 10 equal length parallel lines running across the replica parallel to the lighting direction. The variance in luminance are treated as indicative of the roughness and analyzed by traditional surface roughness statistics.
2) The replica image area is divided into 10 equal width bands and the shadow like features are detected according to their luminance values.

The 8 wrinkle texture parameters measure various aspects of the image produced by the replica surface: Rz and Ra=skin roughness texture; IDL=increases with roughness of the surface; FNum=number of markers indicative of fine and coarse lines per mm; Spacing=mean distance between adjacent strong shadow features; Breadth=proportional to the depth of the wrinkle producing the shadow; Shadows=relative area of shadows cast by all the wrinkles and fine lines; and NumWr=total number of shadowy features available to calculate spacing and breadth.

### Clinical Photography for Visual Evaluation of Fine Lines and Wrinkles Around the Eye Areas, Under-Eye Dark Circles and Under-Eye Puffiness.

Photographs were taken in accordance with regulations provided by consumer protection agencies such as the Federal Trade Commission, the Food and Drug Administration and several other regulatory authorities. The following guidelines were followed: 1) Head position was the same in before and after photos, 2) Same lighting conditions were used and the distance from the camera was same for both, before and after picture, and 3) Same room and background was used for both before and after picture.

Clinical photographs of subjects' faces (frontal, left lateral and right lateral) were taken with Canfield VISTA CR system using parallel polarized mode and UV mode.

Photographs obtained were evaluated for fine lines/wrinkles around the eye areas, under-eye dark circles and under-eye puffiness by a descriptive scale for Evaluation of Photodamage according to the R.W. Johnson Pharmaceutical Research (Griffiths et al., 1992 Arch Dermatol, 128(3): 347-351, incorporated herein by reference) Scale 7-9 listed below: Overall Rating Scale: 0=None, 1-3=Mild, 4-6=Moderate, 7-9=Severe.

### Procedure

1. Subjects reported to the facility; prior to beginning all study related activities subjects completed an informed consent form, photography release form and a medical history form.
2. Subjects were enrolled on the study according to the inclusion/exclusion criteria listed above
3. A minimum of 5 days prior to the start of the study, enrolled subjects began the washout period. Subjects received a neutral soap (Dove soap bar) used for facial cleansing during the entire study period.
4. Subjects were given specific instructions prohibiting use of all cosmetics (exception: lip and eye makeup) and personal care products (e.g., soaps, creams, lotions, masks and any other treatment), on their face for the entire study duration. Subjects were instructed not to begin use of any new products with the exception of products provided by the testing facility for the duration of the study.
5. Following the washout period subjects returned to the facility for baseline measurements.
6. Subjects were instructed to cleanse their face with neutral soap (Dove soap bar) and gently pat dry with paper towel.
7. Thereafter, subjects remained quietly seated for a minimum of 15 minutes in a room maintained at approximately 20-24° C. and approximately 30%-50% relative humidity. Temperature and humidity were recorded during subject testing.
8. The following evaluations were made on their face at baseline (prior to any product treatment):
a. Close-up facial photographs using Standard 1 and parallel polarized modes (frontal, left lateral and right lateral)
b. Skin hydration measurements at sites 1 and 2 (3 readings at each test site)
c. Skin elasticity measurements at sites 1 and 2 (1 reading at each test site)
d. Silicone replicas at sites 1 and 2 (1 replica at each site)
See schematic representation of test sites below.
9. Subjects were assigned to one of the two treatment groups and were provided with the assigned test product along with use instructions as directed by the Sponsor for a period of 8 weeks.

### Use Instructions:

Apply twice daily (once in AM and once in PM) as follows: 1) Wash face with provided bar soap; 2) Gently pat dry; 3) Dispense a dime size amount onto your finger and gently rub in under and to the side of your eye (crow's feet area) until absorbed; 4) Be careful to avoid applying product to your eyelids and eye; 5) Repeat steps 3 and 4 for other eye 10. After 4 weeks (±3 days) and 8 weeks (±3 days) of treatment with the test product subjects were instructed to return to the testing facility. Subjects were instructed not to use the test product until after completion of their scheduled visit. 11. Procedures 6 through 8 were repeated. 12. At the last visit (Week 8) subjects were instructed to complete a post-treatment questionnaire and return any remaining test product to the testing facility.

### Schematic Representation of Test Sites

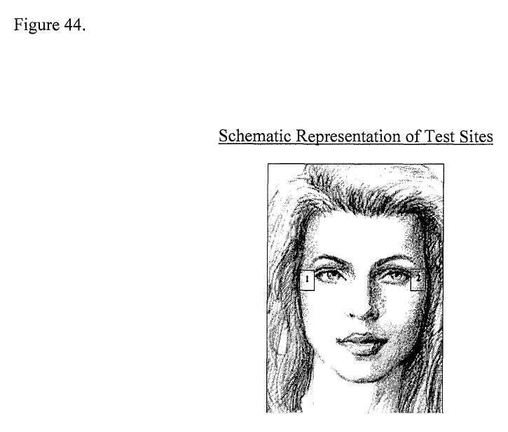

A schematic representation of test sites is shown in FIG. 44.

### Study Results

### Adverse Events

There were no adverse events reported during the study.

### Subjects

Twenty-four female subjects, ranging in age from 35 to 60 years, consented, enrolled and completed the study to assess the efficacy of the test products to improve skin hydration, improve skin elasticity, reduce the appearance of fine lines and wrinkles around the eye areas, decrease the appearance of under-eye dark circles and decrease the appearance of under-eye puffiness.

TABLE 13
Subject Demographics.

| | | | | | Test | | Under |
|---|---|---|---|---|---|---|---|
| | Subject | Subject | | | Product | Dark | Eye |
| No. | ID | Initials | Age | Race | Assigned | Circles | Puffiness |
| 1 | 314 | K L C | 49 | C | A | X | X |
| 2 | 690 | N-L | 44 | H | A | X | X |
| 3 | 893 | A M A | 54 | H | B | X | X |
| 4 | 1298 | L-C | 57 | C | B | X | X |
| 5 | 1521 | K R S | 35 | H | A | | |
| 6 | 1984 | K L S | 53 | AA | A | | X |
| 7 | 2377 | C A H | 56 | C | A | X | |
| 8 | 3153 | M M G | 52 | AA | B | X | X |
| 9 | 4343 | K B M | 45 | C | B | X | X |
| 10 | 4395 | K A F | 52 | C | A | X | X |
| 11 | 4517 | R R M | 60 | AA | A | X | X |
| 12 | 4575 | T L H | 49 | C | B | X | X |
| 13 | 5155 | J J V | 58 | H | B | X | X |
| 14 | 5503 | D J N | 52 | C | B | X | X |
| 15 | 5506 | K S J | 58 | C | B | X | X |
| 16 | 5606 | S-W | 48 | C | B | X | X |
| 17 | 5861 | B E L | 55 | AA | A | X | |
| 18 | 6576 | S R M | 46 | C | A | X | |
| 19 | 6913 | P A S | 59 | C | A | | X |
| 20 | 7005 | W A C | 55 | C | A | X | X |
| 21 | 7633 | S L H | 60 | C | B | | X |
| 22 | 7822 | L R F | 48 | AA | A | X | X |
| 23 | 7832 | C Y K | 59 | C | B | X | X |
| 24 | 8224 | K F J | 53 | C | B | X | X |

| | |
|---|---|
| Average Age | 52.38 |

AA = African American, C = Caucasian, H = Hispanic

### Corneometer Measurements for Skin Hydration

TABLE 14
Mean skin hydration values for Test Products A and B.

| Mean Skin Hydration Values | |
|---|---|
| Test Product A | Test Product B |
| (N = 12) | (N = 12) |

| Interval | Mean | SD | Mean | SD |
|---|---|---|---|---|
| Baseline | 52.56 | 17.26 | 43.77 | 11.66 |
| Week 4 | 57.33 | 15.77 | 50.86 | 12.73 |
| Week 8 | 59.46 | 17.11 | 50.94 | 11.39 |

TABLE 15
Descriptive statistics of skin hydration differences
from baseline for Test Products A and B.

| | | Skin Hydration Differences from Baseline | |
|---|---|---|---|
| | | Test Product | Test Product |
| Interval | Parameter | A | B |
| Week 4 | Mean | 4.77 | 7.09 |
| | SD | 5.28 | 4.66 |
| | % Change | 9.07% | 16.20% |
| | p | <0.05 | <0.001 |
| | % Improvers | 83.33% | 91.67% |
| | P | NS | NS |
| Week 8 | Mean | 6.90 | 7.17 |
| | SD | 5.15 | 5.58 |
| | % Change | 13.13% | 16.38% |
| | p | <0.001 | <0.001 |
| | % Improvers | 83.33% | 91.67% |
| | P | NS | NS |

Note:
Positive difference indicates increased skin hydration.

TABLE 16
Data analysis of skin hydration Test Product A differences from
baseline versus Test Product B differences from baseline.

| Comparison | | | |
|---|---|---|---|
| (% ΔA − % ΔB) | Interval | Variation Mean % | p-value |
| A − B | Week 4 | −7.13% | NS |
| A − B | Week 8 | −3.24% | NS |

Note:
Positive difference indicates Test Product A site more hydrated.
NS = not significant

TABLE 17
Analysis of skin hydration Test Product B differences from
baseline versus Test Product A differences from baseline.

| Comparison | | | |
|---|---|---|---|
| (% ΔB − % ΔA) | Interval | Variation Mean % | p-value |
| B − A | Week 4 | 7.13% | NS |
| B − A | Week 8 | 3.24% | NS |

Note:
Positive difference indicates Test Product B site more hydrated.
NS = not significant

### Cutometer Measurements for Skin Elasticity

TABLE 18
Mean skin elasticity values for Test Products A and B.

| Mean Skin Elasticity Values | |
|---|---|
| Test Product A | Test Product B |
| (N = 12) | (N = 12) |

| Interval | Mean | SD | Mean | SD |
|---|---|---|---|---|
| Baseline | 0.5368 | 0.1191 | 0.5069 | 0.0839 |
| Week 4 | 0.5790 | 0.0852 | 0.5625 | 0.1195 |
| Week 8 | 0.5246 | 0.0736 | 0.5214 | 0.1174 |

TABLE 19
Descriptive statistics of skin elasticity differences
from baseline for Test Products A and B.

| | | Skin Elasticity Differences from Baseline | |
|---|---|---|---|
| | | Test Product | Test Product |
| Interval | Parameter | A | B |
| Week 4 | Mean | 0.0422 | 0.0556 |
| | SD | 0.0765 | 0.1042 |
| | % Change | 7.86% | 10.97% |
| | P | NS | NS |
| | % improvers | 66.67% | 66.67% |
| | P | NS | NS |
| Week 8 | Mean | −0.0122 | 0.0145 |
| | SD | 0.1083 | 0.0848 |
| | % Change | −2.27% | 2.86% |
| | P | NS | NS |
| | % improvers | 50.00% | 75.00% |
| | P | NS | NS |

Note:
Positive difference indicates increased skin elasticity.
Statistically significant values have p < 0.05.
NS = not significant

TABLE 20
Analysis of skin elasticity Test Product A differences from
baseline versus Test Product B differences from baseline.

| Comparison | | | |
|---|---|---|---|
| (% ΔA − % ΔB) | Interval | Variation Mean % | p-value |
| A − B | Week 4 | −3.11% | NS |
| A − B | Week 8 | −5.14% | NS |

Note:
Positive difference indicates Test Product A site more elastic.
NS = not significant

TABLE 21
Analysis of skin elasticity Test Product B differences from
baseline versus Test Product A differences from baseline.

| Comparison | | | |
|---|---|---|---|
| (% ΔB − % ΔA) | Interval | Variation Mean % | p-value |
| B − A | Week 4 | 3.11% | NS |
| B − A | Week 8 | 5.14% | NS |

Note:
Positive difference indicates Test Product B site more elastic.
NS = not significant

### Silicone Replica Analysis for Periocular Wrinkles and Fine Lines

Parameters for Skin Texture:

Rz and Ra=skin roughness texture parameters; decreases in Rz and/or Ra indicate an increase in skin smoothness; IDL=length of line; decrease in IDL indicates an increase in skin smoothness; Shadows=area of shadows cast by all lines; decrease in Shadows indicates an increase in skin smoothness; NumWr=total number of shadowy features; and decrease in NumWr indicates an increase in skin smoothness.

Parameters for Number and Depth of Fine and Coarse Lines:

FNum=number of markers indicative of coarse or fine lines per mm; decrease in FNum indicates a decrease in number of coarse or fine lines; Spacing=mean distance between adjacent strong shadow features; increase in Spacing indicates a decrease in number of coarse or fine lines; Breadth=depth of the wrinkle/line producing shadow; and decrease in Breadth indicates a decrease in depth of coarse or fine lines.

TABLE 22
Data analysis of mean differences from baseline in silicone replica parameters
for Fine Lines after 4 and 8 weeks of treatment with the Test Product A.
Fine Lines (Sample P)

| | | | | Week 4 | | Week 8 | |
|---|---|---|---|---|---|---|---|
| | | | | | Percent | | Percent |
| | Mean | Mean | Mean | Difference | Difference | Difference | Difference |
| | Baseline | Week 4 | Week 8 | From | From | From | From |
| Parameter | Value | Value | Value | Baseline | Baseline | Baseline | Baseline |
| Rz | 118.43 | 120.09 | 130.99 | 1.66 | 1.40% | 12.56 | 10.60% |
| Ra | 24.54 | 24.55 | 27.52 | 0.01 | 0.05% | 2.98 | 12.14% |
| FNUM | 0.67 | 0.56 | 0.54 | −0.11 | −16.65% | −0.14 | −20.19% |
| IDL | 4.74 | 5.05 | 5.51 | 0.32 | 6.71% | 0.77 | 16.29% |
| Spacing | 1.53 | 1.94 | 1.44 | 0.41 | 26.98% | −0.08 | −5.45 |
| Breadth | 0.17 | 0.18 | 0.19 | 0.01 | 6.92% | 0.02 | 14.06% |
| Shadows | 5.52 | 4.90 | 6.50 | −0.62 | −11.18% | 0.98 | 17.82% |
| NumWr | 61.04 | 68.46 | 81.75 | 7.42 | 12.15% | 20.71 | 33.92% |

TABLE 23
Data analysis of mean differences from baseline in silicone replica parameters
for Fine Lines after 4 and 8 weeks of treatment with the Test Product B.
Fine Lines (Sample P)

| | | | | Week 4 | | Week 8 | |
|---|---|---|---|---|---|---|---|
| | | | | | Percent | | Percent |
| | Mean | Mean | Mean | Difference | Difference | Difference | Difference |
| | Baseline | Week 4 | Week 8 | From | From | From | From |
| Parameter | Value | Value | Value | Baseline | Baseline | Baseline | Baseline |
| Rz | 112.31 | 109.02 | 119.21 | −3.29 | −2.93% | 6.90 | 6.15% |
| Ra | 23.80 | 21.85 | 25.23 | −1.96 | −8.22% | 1.43 | 6.01% |
| FNUM | 0.58 | 0.50 | 0.51 | −0.08 | −14.32% | −0.07 | −11.61% |
| IDL | 4.44 | 4.52 | 5.15 | 0.08 | 1.82 | 0.71 | 15.96% |
| Spacing | 1.68 | 1.82 | 1.37 | 0.14 | 8.38% | −0.31 | −18.43% |
| Breadth | 0.18 | 0.18 | 0.20 | 0.005 | 2.53% | 0.02 | 11.40% |
| Shadows | 4.43 | 3.62 | 5.39 | −0.81 | −18.27% | 0.97 | 21.85% |
| NumWr | 46.13 | 50.50 | 69.13 | 4.38 | 9.49% | 23 | 49.86% |

TABLE 24
Data analysis of mean differences from baseline in silicone replica parameters
for Coarse Lines after 4 and 8 weeks of treatment with Test Product A.
For Coarse Lines

| | | | | Week 4 | | Week 8 | |
|---|---|---|---|---|---|---|---|
| | | | | | Percent | | Percent |
| | Mean | Mean | Mean | Difference | Difference | Difference | Difference |
| | Baseline | Week 4 | Week 8 | From | From | From | From |
| Parameter | Value | Value | Value | Baseline | Baseline | Baseline | Baseline |
| Rz | 153.99 | 164.65 | 156.03 | 10.65 | 6.92% | 2.03 | 1.32% |
| Ra | 33.82 | 35.25 | 34.68 | 1.44 | 4.25% | 0.87 | 2.56% |
| FNUM | 0.68 | 0.58 | 0.57 | −0.10 | −14.46% | −0.11 | −16.32% |
| IDL | 6.59 | 7.55 | 6.86 | 0.96 | 14.61% | 0.27 | 4.07% |
| Spacing | 0.96 | 1.01 | 1.12 | 0.05 | 5.56% | 0.16 | 16.23% |
| Breadth | 0.20 | 0.22 | 0.23 | 0.02 | 10.31% | 0.03 | 13.75% |
| Shadows | 10.20 | 10.93 | 9.03 | 0.73 | 7.15% | −1.18 | −11.56% |
| NumWr | 95.08 | 118.33 | 95.92 | 23.25 | 24.45% | 0.83 | 0.88% |

TABLE 25
Data analysis of mean differences from baseline in silicone replica parameters
for Coarse Lines after 4 and 8 weeks of treatment with Test Product B.
For Coarse Lines

| | | | | Week 4 | | Week 8 | |
|---|---|---|---|---|---|---|---|
| | | | | | Percent | | Percent |
| | Mean | Mean | Mean | Difference | Difference | Difference | Difference |
| | Baseline | Week 4 | Week 8 | From | From | From | From |
| Parameter | Value | Value | Value | Baseline | Baseline | Baseline | Baseline |
| Rz | 157.82 | 166.76 | 162.43 | 8.95 | 5.67% | 4.61 | 2.92% |
| Ra | 34.73 | 36.41 | 34.57 | 1.69 | 4.86% | −0.15 | −0.44% |
| FNUM | 0.74 | 0.46 | 0.45 | −0.27 | −36.94% | −0.28 | −38.43% |
| IDL | 6.19 | 7.36 | 6.55 | 1.17 | 18.83% | 0.36 | 5.76% |
| Spacing | 1.12 | 1.05 | 1.35 | −0.07 | −6.54% | 0.23 | 20.82% |
| Breadth | 0.25 | 0.25 | 0.28 | 0.003 | 1.04% | 0.03 | 13.18% |
| Shadows | 12.26 | 11.63 | 11.35 | −0.63 | −5.13% | −0.91 | −7.41% |
| NumWr | 81.21 | 105.29 | 94.08 | 24.08 | 29.66% | 12.88 | 15.85% |

TABLE 26
Percentage of subjects showing improvement.

| | Test Product A | | | | Test Product B | | | |
|---|---|---|---|---|---|---|---|---|
| | Fine Lines | | Coarse Lines | | Fine Lines | | Coarse Lines | |
| | Week | Week | Week | Week | Week | Week | Week | Week |
| Parameter | 4 | 8 | 4 | 8 | 4 | 8 | 4 | 8 |
| Rz | 41.67% | 16.67% | 41.67% | 41.67% | 50% | 33.33% | 33.33% | 50% |
| Ra | 50% | 33.33% | 33.33% | 50% | 66.67% | 50% | 41.67% | 58.33% |
| FNUM | 100% | 91.67% | 91.67% | 91.67% | 83.33% | 83.33% | 83.33% | 91.67% |
| IDL | 16.67 | 25% | 25% | 41.67% | 58.33% | 16.67% | 8.33% | 25% |
| Spacing | 83.33% | 50% | 50% | 66.67% | 66.67% | 33.33% | 50% | 66.67% |
| Breadth | 8.33% | 8.33% | 41.67% | 33.33% | 33.33% | 25% | 50% | 25% |
| Shadows | 58.33% | 41.67% | 58.33% | 50% | 58.33% | 41.67% | 50% | 61.67% |
| NumWr | 50% | 8.33% | 8.33% | 50% | 41.67% | 8.33% | 8.33% | 33.33% |

### Visual Evaluations of Clinical Photographs

TABLE 27
Descriptive statistics of mean differences from baseline for
evaluations of clinical photographs for Test Product A.

| | Week 4 | | | Week 8 | | |
|---|---|---|---|---|---|---|
| | Fine Lines/ | Under Eye | | Fine Lines/ | Under Eye | |
| | Wrinkles | Puffiness | Dark Circles | Wrinkles | Puffiness | Dark Circles |
| Parameter | (N = 12) | (N = 8) | (N = 9) | (N = 12) | (N = 8) | (N = 9) |
| Mean | −0.17 | 0.13 | −0.22 | −0.25 | −0.38 | −0.44 |
| Difference | | | | | | |
| SD | 0.72 | 0.35 | 0.44 | 0.97 | 0.52 | 0.53 |
| p-value | NS | NS | NS | NS | NS | NS |
| Percent of | 33.33% | 0.00% | 22.22% | 25.00% | 37.50% | 44.44% |
| Subjects | | | | | | |
| Improving | | | | | | |
| p-value | NS | NS | NS | NS | NS | NS |

Note:
Negative differences indicate improvementin parameter.
NS = Not Significant

TABLE 28
Descriptive statistics of mean differences from baseline for
evaluations of clinical photographs
for Test Product B.

| | Week 4 | | | Week 8 | | |
|---|---|---|---|---|---|---|
| | Fine Lines/ | Under Eye | | Fine Lines/ | Under Eye | |
| | Wrinkles | Puffiness | Dark Circles | Wrinkles | Puffiness | Dark Circles |
| Parameter | (N = 12) | (N = 12) | (N = 11) | (N = 12) | (N = 12) | (N =11) |
| Mean | −0.50 | −0.08 | −0.09 | −0.58 | −0.17 | −0.36 |
| Difference | | | | | | |
| SD | 0.52 | 0.29 | 0.70 | 0.79 | 0.58 | 0.50 |
| p-value | <0.05 | NS | NS | NS | NS | NS |
| Percent of | 50.00% | 8.33% | 9.09% | 41.67% | 25.00% | 36.36% |
| Subjects | | | | | | |
| Improving | | | | | | |
| p-value | NS | NS | NS | NS | NS | NS |

Note:
Negative differences indicate improvement in parameter.
Statistically significant values have p < 0.05.
NS = Not Significant

TABLE 29
Analysis of comparisons of mean visual evaluations differences from
baseline for each treatment group at each post-treatment interval.

| | | Test Product A | Test Product B |
|---|---|---|---|
| 1 Parameter | Interval | A − B | B − A |
| Fine Lines/ | Week 4 | 0.33 | −0.33 |
| Wrinkles | p-value | NS | NS |
| | Week 8 | 0.33 | −0.33 |
| | p-value | NS | NS |
| Under Eye | Week 4 | 0.21 | −0.21 |
| Puffiness | p-value | NS | NS |
| | Week 8 | −0.21 | 0.21 |
| | p-value | NS | NS |
| Dark Circles | Week 4 | −0.13 | 0.13 |
| | p-value | NS | NS |
| | Week 8 | −0.08 | 0.08 |
| | p-value | NS | NS |

Note:
Negative differences under “Test Product” column head indicate greater improvement for test product compared to test product in sub-column head.
NS = Not Significant

Subject Evaluations from Post-Treatment Questionnaires

TABLE 30
Analysis of subjects' responses from each treatment group
for the following questions 8 weeks post-treatment.
Scale: 4 = Strongly Agree, 3 = Agree, 2 =
Disagree, 1 = Strongly Disagree

| | Percent of Subjects “Agreeing” | |
|---|---|---|
| | Product A | Product B |
| Statement | (N = 12) | (N = 12) |
| The skin around my eyes feels more | 75.00% | 66.67% |
| hydrated/moisturized. | | |
| The fine lines/wrinkles around my | 66.67% | 58.33% |
| eyes are less visible. | | |
| The skin around my eyes feels more | 75.00% | 75.00% |
| toned/firmer. | | |
| The skin around my eyes is less puffy. | 83.33% | 66.67% |
| The dark circles under my eyes are | 75.00% | 58.33% |
| less visible. | | |
| The skin around my eyes feels | 91.67% | 83.33% |
| smoother. | | |

Bold Values indicate statistical significance <0.05

The raw data for the experiments detailed above is provided in the tables below.

TABLE 31
(A) Raw Data for Corneometer for Product A.

| | | | Baseline | | Week 4 | | Week 8 | |
|---|---|---|---|---|---|---|---|---|
| Subject | Subject | | Right | Left | Right | Left | Right | Left |
| ID | Initials | Replicate | PA | PA | PA | PA | PA | PA |
| 314 | KLC | 1 | 24.5 | 32.2 | 27.2 | 32.9 | 25.9 | 33.5 |
| | | 2 | 27.1 | 35.2 | 24.6 | 34.6 | 26.2 | 34.2 |
| | | 3 | 28.9 | 36.7 | 26.6 | 35.5 | 28.2 | 35.3 |
| 690 | N-L | 1 | 64.8 | 57.6 | 69.5 | 63.0 | 73.4 | 69.1 |
| | | 2 | 64.0 | 59.8 | 70.5 | 64.4 | 75.5 | 73.2 |
| | | 3 | 66.3 | 62.4 | 72.4 | 64.9 | 76.6 | 74.7 |
| 1521 | KRS | 1 | 49.4 | 47.4 | 57.4 | 49.2 | 57.6 | 49.6 |
| | | 2 | 48.0 | 49.9 | 59.5 | 55.6 | 61.3 | 56.3 |
| | | 3 | 49.1 | 50.3 | 58.8 | 54.3 | 59.1 | 52.4 |
| 1984 | KLS | 1 | 87.5 | 78.1 | 83.0 | 74.5 | 96.8 | 85.1 |
| | | 2 | 87.2 | 78.9 | 84.8 | 75.5 | 98.4 | 86.4 |
| | | 3 | 89.9 | 80.2 | 86.4 | 75.3 | 99.1 | 87.5 |
| 2377 | CAH | 1 | 65.0 | 57.3 | 67.8 | 61.5 | 65.6 | 58.1 |
| | | 2 | 67.9 | 59.9 | 69.9 | 63.9 | 67.4 | 59.1 |
| | | 3 | 70.1 | 60.4 | 69.0 | 65.2 | 68.0 | 60.3 |
| 4395 | KAF | 1 | 23.9 | 26.0 | 26.7 | 31.0 | 35.3 | 30.5 |
| | | 2 | 17.2 | 28.1 | 28.5 | 28.9 | 39.1 | 33.3 |
| | | 3 | 20.7 | 26.1 | 26.0 | 31.3 | 36.7 | 34.7 |
| 4517 | RRM | 1 | 63.4 | 59.8 | 68.0 | 70.7 | 70.4 | 69.0 |
| | | 2 | 66.7 | 60.6 | 70.6 | 72.6 | 75.2 | 70.8 |
| | | 3 | 68.2 | 62.1 | 71.6 | 75.5 | 73.1 | 73.3 |
| 5861 | BEL | 1 | 62.1 | 63.2 | 62.1 | 68.2 | 62.2 | 69.9 |
| | | 2 | 63.5 | 67.2 | 63.1 | 69.7 | 65.2 | 69.1 |
| | | 3 | 64.2 | 64.2 | 63.3 | 67.1 | 67.8 | 69.1 |
| 6576 | SRM | 1 | 51.4 | 68.1 | 57.4 | 69.3 | 58.2 | 69.3 |
| | | 2 | 53.9 | 68.1 | 58.9 | 71.2 | 60.5 | 70.8 |
| | | 3 | 56.1 | 69.2 | 60.4 | 72.0 | 61.0 | 71.6 |
| 6913 | PAS | 1 | 54.0 | 51.6 | 57.7 | 55.6 | 57.9 | 57.4 |
| | | 2 | 56.2 | 51.6 | 58.9 | 59.2 | 58.2 | 58.6 |
| | | 3 | 57.2 | 51.2 | 61.3 | 58.0 | 60.1 | 62.4 |
| 7005 | WAC | 1 | 36.3 | 40.5 | 39.0 | 51.7 | 41.5 | 48.5 |
| | | 2 | 35.6 | 43.2 | 38.8 | 52.4 | 40.3 | 49.3 |
| | | 3 | 37.5 | 41.7 | 40.1 | 47.6 | 40.9 | 51.1 |
| 7822 | LRF | 1 | 37.7 | 34.7 | 58.4 | 51.1 | 53.0 | 52.2 |
| | | 2 | 38.5 | 35.0 | 55.0 | 52.0 | 55.4 | 53.0 |
| | | 3 | 39.9 | 32.0 | 55.9 | 53.3 | 56.1 | 55.5 |

TABLE 32
(B) Raw Data for Corneometer for Product B. (N = 12).

| | | | Baseline | | Week 4 | | Week 8 | |
|---|---|---|---|---|---|---|---|---|
| Subject | Subject | | Right | Left | Right | Left | Right | Left |
| ID | Initials | Replicate | PA | PA | PA | PA | PA | PA |
| 893 | AMA | 1 | 41.3 | 38.4 | 59.9 | 47.1 | 54.0 | 48.2 |
| | | 2 | 44.0 | 37.7 | 60.3 | 46.8 | 54.9 | 47.4 |
| | | 3 | 45.7 | 40.9 | 55.3 | 49.3 | 57.2 | 51.5 |
| 1298 | L-C | 1 | 35.4 | 38.1 | 45.8 | 40.7 | 41.4 | 41.7 |
| | | 2 | 36.3 | 38.3 | 46.7 | 43.1 | 43.4 | 43.4 |
| | | 3 | 36.0 | 39.9 | 48.6 | 43.0 | 40.6 | 41.9 |
| 3153 | MMG | 1 | 59.1 | 55.5 | 64.3 | 66.0 | 66.0 | 72.8 |
| | | 2 | 64.0 | 56.9 | 67.9 | 70.3 | 68.3 | 73.9 |
| | | 3 | 66.2 | 58.2 | 69.3 | 67.7 | 69.9 | 74.2 |
| 4343 | KBM | 1 | 50.4 | 55.0 | 63.1 | 61.7 | 51.9 | 51.1 |
| | | 2 | 52.4 | 59.1 | 65.9 | 65.4 | 55.7 | 54.4 |
| | | 3 | 55.3 | 60.2 | 64.4 | 60.1 | 51.3 | 57.8 |
| 4575 | TLH | 1 | 10.8 | 21.6 | 18.5 | 26.5 | 30.4 | 32.9 |
| | | 2 | 11.4 | 17.4 | 21.2 | 29.9 | 30.3 | 33.7 |
| | | 3 | 10.4 | 21.2 | 16.5 | 28.6 | 31.6 | 33.3 |
| 5155 | JJV | 1 | 47.6 | 49.9 | 66.4 | 59.8 | 64.3 | 58.4 |
| | | 2 | 48.2 | 52.9 | 69.0 | 60.4 | 66.7 | 58.6 |
| | | 3 | 48.8 | 50.6 | 69.8 | 61.0 | 68.1 | 59.0 |
| 5503 | DJN | 1 | 41.1 | 40.7 | 38.3 | 44.6 | 39.1 | 45.7 |
| | | 2 | 38.0 | 42.7 | 42.5 | 40.1 | 39.6 | 46.8 |
| | | 3 | 41.9 | 43.0 | 41.1 | 42.7 | 42.3 | 46.6 |
| 5506 | KSJ | 1 | 44.4 | 48.8 | 55.3 | 58.7 | 60.9 | 56.6 |
| | | 2 | 45.9 | 51.0 | 57.6 | 61.3 | 61.7 | 57.5 |
| | | 3 | 45.2 | 49.9 | 59.2 | 60.6 | 61.8 | 58.2 |
| 5606 | S-W | 1 | 35.2 | 30.6 | 38.3 | 36.1 | 35.1 | 36.3 |
| | | 2 | 32.9 | 31.1 | 35.9 | 40.1 | 34.6 | 35.2 |
| | | 3 | 36.1 | 32.1 | 35.9 | 38.0 | 33.3 | 38.5 |
| 7633 | SLH | 1 | 45.2 | 42.6 | 57.8 | 54.9 | 50.6 | 51.1 |
| | | 2 | 47.6 | 43.0 | 53.3 | 55.1 | 51.0 | 50.9 |
| | | 3 | 48.4 | 44.0 | 53.6 | 54.1 | 51.7 | 52.7 |
| 7832 | CYK | 1 | 45.8 | 46.9 | 47.8 | 49.1 | 47.5 | 47.5 |
| | | 2 | 47.9 | 48.4 | 46.6 | 45.0 | 47.3 | 48.6 |
| | | 3 | 49.4 | 47.2 | 48.1 | 45.6 | 49.3 | 51.3 |
| 8224 | KFJ | 1 | 48.6 | 50.7 | 55.6 | 49.5 | 63.0 | 53.6 |
| | | 2 | 51.1 | 52.4 | 57.7 | 47.7 | 64.1 | 56.9 |
| | | 3 | 53.4 | 53.2 | 60.9 | 53.1 | 65.4 | 55.1 |

TABLE 33
(C) Raw data for Cutometer (Elasticity) for Product A. (N = 12).

| | | Baseline | | Week 4 | | Week 8 | |
|---|---|---|---|---|---|---|---|
| Subject | Subject | Right | Left | Right | Left | Right | Left |
| ID | Initials | PA | PA | PA | PA | PA | PA |
| 314 | KLC | 0.7158 | 0.6536 | 0.6219 | 0.5237 | 0.4825 | 0.5268 |
| 690 | N-L | 0.5792 | 0.3420 | 0.5829 | 0.3771 | 0.5159 | 0.4082 |
| 1521 | KRS | 0.5471 | 0.6708 | 0.5666 | 0.6502 | 0.4734 | 0.5115 |
| 1984 | KLS | 0.5844 | 0.7261 | 0.6605 | 0.6812 | 0.6193 | 0.7389 |
| 2377 | CAH | 0.4582 | 0.5366 | 0.6274 | 0.6905 | 0.5193 | 0.4686 |
| 4395 | KAF | 0.7200 | 0.8157 | 0.7403 | 0.7393 | 0.5621 | 0.6395 |
| 4517 | RRM | 0.4471 | 0.5963 | 0.5374 | 0.7188 | 0.4473 | 0.5333 |
| 5861 | BEL | 0.3644 | 0.6122 | 0.6203 | 0.4916 | 0.6306 | 0.6263 |
| 6576 | SRM | 0.3819 | 0.5263 | 0.5000 | 0.6203 | 0.4566 | 0.4895 |
| 6913 | PAS | 0.4244 | 0.3029 | 0.4929 | 0.4187 | 0.3704 | 0.5781 |
| 7005 | WAC | 0.4089 | 0.4232 | 0.4762 | 0.5520 | 0.5662 | 0.5304 |
| 7822 | LRF | 0.5118 | 0.5351 | 0.5618 | 0.4451 | 0.4278 | 0.4684 |

TABLE 34
(D) Raw data for Cutometer (Elasticity) for Product B. (N = 12).

| | | Baseline | | Week 4 | | Week 8 | |
|---|---|---|---|---|---|---|---|
| Subject | Subject | Right | Left | Right | Left | Right | Left |
| ID | Initials | PA | PA | PA | PA | PA | PA |
| 893 | AMA | 0.4468 | 0.4273 | 0.4134 | 0.4686 | 0.4281 | 0.4563 |
| 1298 | L-C | 0.6205 | 0.6982 | 0.7095 | 0.6031 | 0.6680 | 0.7035 |
| 3153 | MMG | 0.5191 | 0.6226 | 0.7122 | 0.7470 | 0.5456 | 0.6633 |
| 4343 | KBM | 0.5616 | 0.5594 | 0.5099 | 0.4327 | 0.5069 | 0.4822 |
| 4575 | TLH | 0.5104 | 0.4481 | 0.7385 | 0.7118 | 0.6109 | 0.6202 |
| 5155 | JJV | 0.4382 | 0.4542 | 0.3310 | 0.4343 | 0.3307 | 0.3689 |
| 5503 | DJN | 0.6151 | 0.5177 | 0.6331 | 0.5184 | 0.4044 | 0.7401 |
| 5506 | KSJ | 0.4098 | 0.5729 | 0.4796 | 0.4511 | 0.3651 | 0.3509 |
| 5606 | S-W | 0.6024 | 0.5892 | 0.7409 | 0.6679 | 0.5730 | 0.6844 |
| 7633 | SLH | 0.3676 | 0.5017 | 0.5699 | 0.6228 | 0.4419 | 0.4936 |
| 7832 | CYK | 0.3438 | 0.3864 | 0.5342 | 0.4773 | 0.3352 | 0.4626 |
| 8224 | KFJ | 0.3564 | 0.5964 | 0.6151 | 0.3786 | 0.7500 | 0.5273 |

TABLE 35
(E) Raw Data for Visual Grading of Fine Lines and Wrinkles
(Crow's Feet) for Product A. (N = 12).
Scale: 0 = None, 1-3 = Mild, 4-6 =
Moderate, 7-9 = Severe

| Subject | Subject | | | |
|---|---|---|---|---|
| ID | Initials | Baseline | Week 4 | Week 8 |
| 314 | K L C | 5 | 4 | 3 |
| 690 | N-L | 1 | 1 | 1 |
| 1521 | K R S | 1 | 1 | 1 |
| 1984 | K L S | 2 | 2 | 2 |
| 2377 | C A H | 6 | 5 | 4 |
| 4395 | K A F | 2 | 3 | 3 |
| 4517 | R R M | 1 | 1 | 2 |
| 5861 | B E L | 1 | 1 | 1 |
| 6576 | S R M | 3 | 4 | 2 |
| 6913 | P A S | 8 | 7 | 8 |
| 7005 | W A C | 5 | 4 | 5 |
| 7822 | L R F | 2 | 2 | 2 |

TABLE 36
(F) Raw Data for Visual Grading of Fine Lines and Wrinkles
(Crow's Feet) for Product B. (N = 12).
Scale: 0 = None, 1-3 = Mild, 4-6 =
Moderate, 7-9 = Severe

| Subject | Subject | | | |
|---|---|---|---|---|
| ID | Initials | Baseline | Week 4 | Week 8 |
| 893 | A M A | 4 | 4 | 4 |
| 1298 | L-C | 5 | 4 | 5 |
| 3153 | M M G | 2 | 1 | 1 |
| 4343 | K B M | 2 | 2 | 2 |
| 4575 | T L H | 3 | 3 | 3 |
| 5155 | J J V | 7 | 6 | 5 |
| 5503 | D J N | 7 | 6 | 6 |
| 5506 | K S J | 7 | 6 | 5 |
| 5606 | S-W | 5 | 4 | 5 |
| 7633 | S L H | 6 | 6 | 5 |
| 7832 | C Y K | 5 | 5 | 5 |
| 8224 | K F J | 3 | 3 | 3 |

TABLE 37
(G) Raw Data for Visual Grading of Under Eye
Puffiness for Product A. (N = 8).
Scale: 0 = None, 1-3 = Mild, 4-6 =
Moderate, 7-9 = Severe

| Subject | Subject | | | |
|---|---|---|---|---|
| ID | Initials | Baseline | Week 4 | Week 8 |
| 314 | K L C | 3 | 3 | 2 |
| 690 | N-L | 4 | 4 | 3 |
| 1984 | K L S | 2 | 2 | 2 |
| 4395 | K A F | 3 | 3 | 3 |
| 4517 | R R M | 4 | 4 | 3 |
| 6913 | P A S | 6 | 6 | 6 |
| 7005 | W A C | 2 | 2 | 2 |
| 7822 | L R F | 2 | 3 | 2 |

TABLE 38
(H) Raw Data for Visual Grading of Under Eye
Puffiness for Product B. (N = 12).
Scale: 0 = None, 1-3 = Mild, 4-6 =
Moderate, 7-9 = Severe

| Subject | Subject | | | |
|---|---|---|---|---|
| ID | Initials | Baseline | Week 4 | Week 8 |
| 893 | A M A | 3 | 3 | 3 |
| 1298 | L-C | 5 | 4 | 4 |
| 3153 | M M G | 2 | 2 | 2 |
| 4343 | K B M | 3 | 3 | 3 |
| 4575 | T L H | 4 | 4 | 3 |
| 5155 | J J V | 2 | 2 | 3 |
| 5503 | D J N | 2 | 2 | 2 |
| 5506 | K S J | 3 | 3 | 3 |
| 5606 | S-W | 2 | 2 | 2 |
| 7633 | S L H | 4 | 4 | 4 |
| 7832 | C Y K | 3 | 3 | 3 |
| 8224 | K F J | 3 | 3 | 2 |

TABLE 39
(I) Raw Data for Visual Grading of Dark Circles for Product A. (N = 9).
Scale: 0 = None, 1-3 = Mild, 4-6 = Moderate, 7-9 = Severe

| Subject | Subject | | | |
|---|---|---|---|---|
| ID | Initials | Baseline | Week 4 | Week 8 |
| 314 | K L C | 5 | 4 | 4 |
| 690 | N-L | 4 | 4 | 4 |
| 2377 | C A H | 3 | 3 | 2 |
| 4395 | K A F | 2 | 2 | 2 |
| 4517 | R R M | 6 | 5 | 5 |
| 5861 | B E L | 4 | 4 | 4 |
| 6576 | S R M | 2 | 2 | 1 |
| 7005 | W A C | 2 | 2 | 2 |
| 7822 | L R F | 2 | 2 | 2 |

TABLE 40
(J) Raw Data for Visual Grading of Dark Circles for Product B. (N = 11).
Scale: 0 = None, 1-3 = Mild, 4-6 = Moderate, 7-9 = Severe

| Subject | Subject | | | |
|---|---|---|---|---|
| ID | Initials | Baseline | Week 4 | Week 8 |
| 893 | A M A | 4 | 4 | 4 |
| 1298 | L-C | 5 | 5 | 4 |
| 3153 | M M G | 4 | 4 | 4 |
| 4343 | K B M | 3 | 3 | 3 |
| 4575 | T L H | 4 | 4 | 3 |
| 5155 | J J V | 3 | 3 | 3 |
| 5503 | D J N | 6 | 6 | 5 |
| 5506 | K S J | 3 | 3 | 3 |
| 5606 | S-W | 5 | 3 | 4 |
| 7832 | C Y K | 3 | 4 | 3 |
| 8224 | K F J | 3 | 3 | 3 |

TABLE 41
(K) Raw Data for Subject Post-treatment Questionnaire
for Product A. (N = 12).

| | The skin | The fine | | | | |
|---|---|---|---|---|---|---|
| | around | lines/ | The skin | | The dark | The skin |
| | my | wrinkles | around | The skin | circles | around |
| | eyes feels | around | my | under | under | my |
| | more | my | eyes feels | my | my | eyes |
| | hydrated/ | eyes are | more | eyes are | eyes are | feels |
| Subject | mois- | less | toned/ | less | less | smooth- |
| ID | turized. | visible. | firmer. | puffy. | visible. | er. |
| 314 | 2 | 2 | 3 | 2 | 2 | 3 |
| 690 | 3 | 3 | 2 | 3 | 2 | 3 |
| 1521 | 3 | 2 | 3 | 3 | 2 | 3 |
| 1984 | 3 | 3 | 3 | 3 | 3 | 3 |
| 2377 | 3 | 2 | 2 | 3 | 3 | 3 |
| 4395 | 3 | 3 | 3 | 3 | 3 | 3 |
| 4517 | 2 | 3 | 3 | 4 | 3 | 3 |
| 5861 | 3 | 3 | 3 | 3 | 3 | 3 |
| 6576 | 4 | 4 | 4 | 4 | 4 | 4 |
| 6913 | 4 | 3 | 3 | 2 | 3 | 3 |
| 7005 | 2 | 2 | 2 | 3 | 3 | 2 |
| 7822 | 3 | 3 | 3 | 3 | 3 | 3 |

Scale:
4 = Strongly Agree,
3-Agree,
2 = Disagree,
1 = Strongly Disagree

TABLE 42
(L) Raw Data for Subject Post-treatment Questionnaire
for Product B. (N = 12)

| | The skin | The fine | The skin | | The dark | |
|---|---|---|---|---|---|---|
| | around my | lines/ | around | The skin | circles | The skin |
| | eyes feels | wrinkles | my | under | under | around |
| | more | around my | eyes feels | my | my | my eyes |
| Sub- | hydrated/ | eyes are | more | eyes are | eyes are | feels |
| ject | mois- | less | toned/ | less | less | smooth- |
| ID | turized. | visible. | firmer. | puffy. | visible. | er. |
| 893 | 4 | 3 | 3 | 3 | 3 | 3 |
| 1298 | 3 | 2 | 3 | 2 | 2 | 3 |
| 3153 | 4 | 3 | 3 | 3 | 3 | 3 |
| 4343 | 2 | 3 | 3 | 3 | 3 | 4 |
| 4575 | 3 | 3 | 3 | 2 | 2 | 3 |
| 5155 | 2 | 2 | 2 | 2 | 2 | 2 |
| 5503 | 4 | 2 | 3 | 3 | 3 | 3 |
| 5506 | 3 | 3 | 3 | 3 | 3 | 3 |
| 5606 | 2 | 2 | 2 | 3 | 2 | 3 |
| 7633 | 4 | 3 | 4 | 3 | 3 | 4 |
| 7832 | 3 | 3 | 3 | 3 | 4 | 3 |
| 8224 | 1 | 1 | 1 | 1 | 1 | 2 |

Scale:
4 = Stron l Agree,
3-Agree,
2 = Disagree,
1 = Strongly Disagree

### Example 5

### Bacillus Coagulans Dried Supernatant

As described below, the effect of drying and subsequent rehydration of Bacillus coagulans (BC) supernatant and cell wall fractions, as well as further fractionation based on molecular weight ranges was examined. As described in detail below, gel electrophoresis was performed to compare the crude preparations of BC cell wall and supernatant fractions. Moreover, three different molecular weight ranges from supernatant and cell wall fractions were evaluated in selected bioassays to identify which compounds may be associated with biological activity. The effect of crude cell wall and supernatant fractions of BC on dendritic cell maturation was examined. Finally, as described in detail below, it was determined if selected key biological activities of the BC supernatant and cell wall fractions is preserved after drying and rehydration.

Described herein are anti-inflammatory compound(s) present in the high molecular weight fraction (30-200 kDa) of BC30. As described below, both high- and low-molecular weight immune modulating compound(s) present in BC30 fractions activate NK cells. Additionally, compounds, particularly compounds in the metabolite fraction, trigger induction of IL-6 and TNF-alpha.

As described below, drying and reconstituting Bacillus coagulans extracellular product (metabolites/supernatant) results in unexpected anti-inflammatory effects. Drying the Bacillus coagulans extracellular product (metabolites/supernatant) inactivated or removed undesirable compounds (e.g., volatile organic compounds) that would otherwise inhibit the anti-inflammatory effects of the Bacillus coagulans extracellular product. For example, drying and reconstituting the Bacillus coagulans extracellular product results in at least 1% greater anti-inflammatory activity compared to Bacillus coagulans extracellular product alone, e.g., at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% greater anti-inflammatory activity compared to Bacillus coagulans extracellular product alone.

### Fractionation of GBI-30 (GB-30/Ganeden BC³⁰™/BC³⁰)

The test fractions of Bacillus coagulans (BC30) supernatant (metabolites fraction) and cell wall were prepared as follows. A sample of Bacillus coagulans spores was heat-activated at 50° C. and inoculated in liquid culture medium. The sample was incubated at 37° C. for 24 hours at which time additional media was added followed by incubation at 37° C. for an additional 24 hours. This time period allowed the formation of a log-phase bacterial culture where death and bacterial breakdown was not prominent. After the incubation, the two fractions (Bacillus coagulans supernatant (BC1) and Bacillus coagulans cell wall components (BC2)) were prepared. The initial separation occurred by decanting the entire culture into a 50 mL vial followed by centrifugation at 2400 rpm. This resulted in the bacteria forming a pellet. The supernatant was gently decanted into a new vial. From this vial, smaller 1 mL samples were aliquoted into Eppendorf vials and subjected to high speed centrifugation, followed by two serial filtrations with a 0.2 um filter, to eliminate any intact bacteria and fractions thereof. The sterile, filtered supernatant was aliquoted and multiple aliquots frozen and stored at −20° C. For later biological assays, one aliquot was thawed on each testing day.

The original pellet from the initial centrifugation was used to prepare the cell wall fraction. The bacterial pellet was washed twice with physiological saline, and the wet pellet was frozen and thawed several times to break open the bacterial walls so that the intracellular compounds could be removed by washing. The thawed slush was transferred to a glass vial and subjected to multiple rounds of bead milling using low-protein-binding Zirconium beads with a diameter of 100 micrometer. The milling was performed by repeated ‘pulsing’ using a Vortex mixer. This method is effective to break up cell walls of bacteria and cyanobacteria. The beads were removed and the slush containing the broken cell wall fragments were sterile-filtered into multiple aliquots that were frozen immediately and stored at −20° C. For later assays, one aliquot was thawed on each testing day. Similar volumes of Bacillus Coagulans cell wall or supernatant were placed into centrifugation columns that filter out specific molecular weight fractions. After centrifugation, the remaining volumes were serial diluted and used in downstream bioassays.

### Example 6

### Electrophoresis of Bacillus coagulans Supernatant and Cell Wall Components

In order to identify the molecular weights of predominant protein/carbohydrate compounds in the supernatant and cell wall fractions of BC, electrophoresis was used to understand the protein and polysaccharide makeup of BC30 (GBI-30/GB-30/Ganeden BC³⁰™/BC³⁰, ATCC Designation Number PTA-6086) fractions and spores. Crude BC cell wall and supernatant (extracellular product/metabolite) fractions were further separated into three different size ranges using molecular weight cutoff filters.

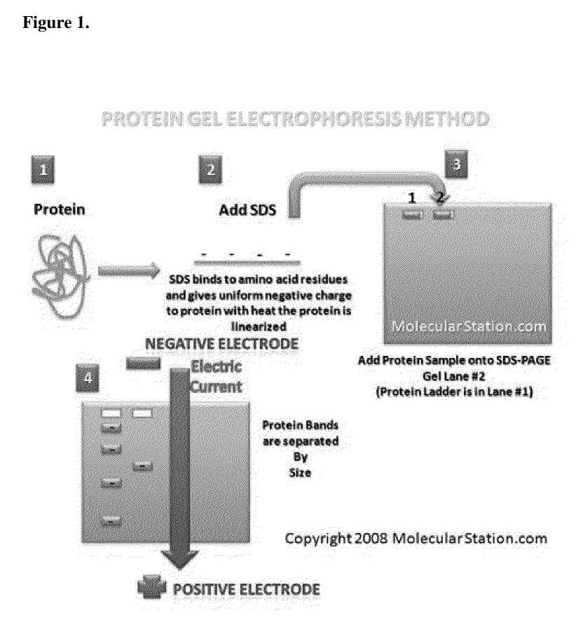

A typical protein gel electrophoresis method is shown in FIG. 1. This process separates the proteins and polysaccharides by molecular weight and gives a valuable fingerprint for each of the BC fractions. Electrophoretic separation provides information about the relative quantity of specific proteins and polysaccaharides in the product.

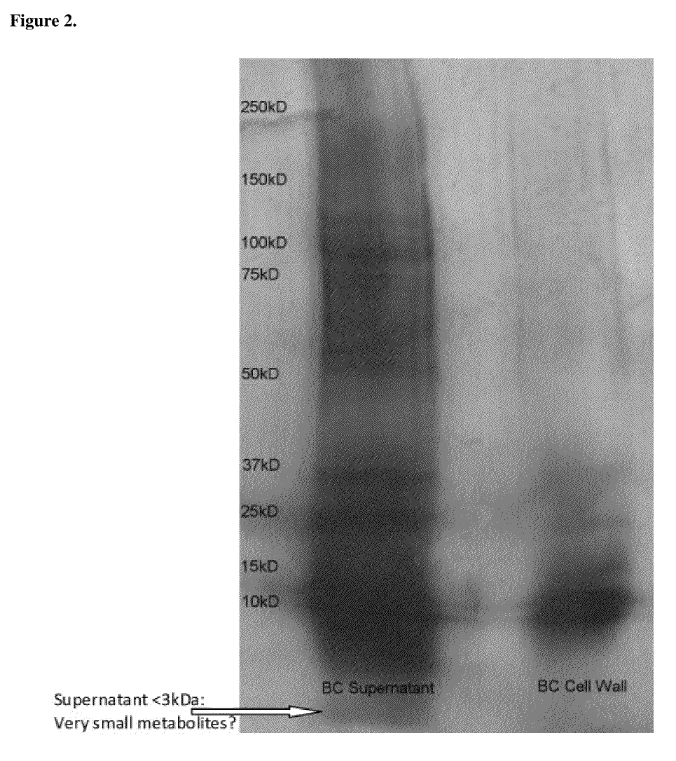

Gel electrophoresis of the previous batch of supernatant and cell wall fractions showed several regions of interest. The supernatant contained compounds lower than 5-10 kDa, i.e., lower than the range that can be clearly fractionated by SDS gel electrophoresis (see, smear below the words “BC Supernatant” in FIG. 2). Both fractions contained double bands in the 10 kDa range. The supernatant contained several additional prominent bands between 20-30 kDa and between 50-150 kDa. The dual bands seen in both cell wall and supernatant fractions may be identical compounds, and therefore be responsible for the same biological activities. However, the additional prominent compounds in the supernatant may possess other biological activities.

Fractionation of the BC³⁰ crude supernatant (metabolites) and the crude cell wall fraction was carried out to yield three fractions or purified preparations A) below 3 kDa, B) between 3-30 kDa, and C) between 30-200 kDa. The major bioactive compounds from the cell wall are in fraction B. Electrophoresis is used as a tool to ensure product consistency during stages of product development. It is also useful as a regular quality control tool during manufacturing.

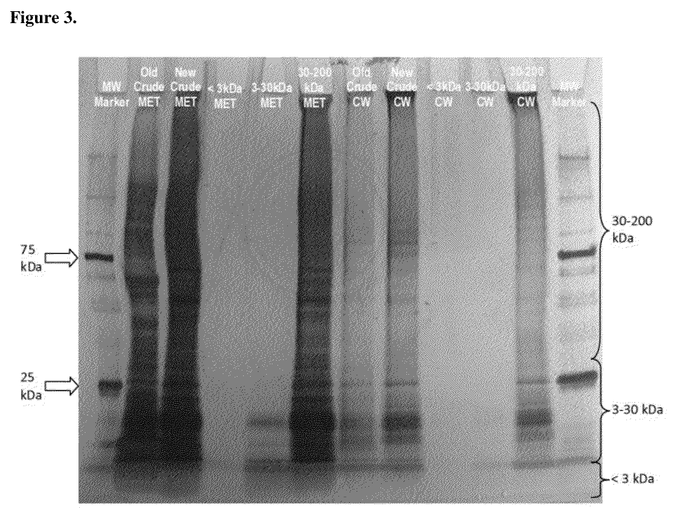

As shown in FIG. 3, gel electrophoresis shows that the preparations of BC30 supernatant (metabolites) and cell wall fractions are concentrated, and confirms the presence of compounds in the BC crude cell wall and metabolite fractions. FIG. 3 also shows the results of the fractionation steps where only very small compounds are seen in the <3 kDa lanes, slightly larger compounds are recovered in the 3-30 kDa fraction lanes, and the 30-200 kDa fractions are most similar to the un-fractionated crude metabolite and cell wall preparations. Many similar sized compounds are shared between the crude metabolite and crude cell wall preparations. This could be due to identical compounds in the two fractions or different compounds that have the same molecular weight. The biggest difference between the crude metabolite and crude cell wall fractions is the presence of more bands in the metabolite fraction, particularly in the size range between 75 kDa and 25 kDa (2 darkest bands seen in the MW marker lanes).

As described in detail below, size fractionation by molecular weight (<3, 3-30, 30-200 kDa) of both supernatant and cell wall fractions was performed to further characterize the following three biological activities: a) Anti-inflammatory effect, as measured by inhibition of cell migration in response to inflammatory mediators; b) Effect on NK cell activation; and c) Effect on cytokine production.

### Example 7

### Anti-Inflammatory Effect: Inhibition of Leukotriene B4 Directed Migration

The polymorphonuclear leukocyte (PMN) cell is a highly active and migratory cell type. Bacillus coagulans fractions have strong anti-inflammatory effects when exposed to the known inflammatory cytokine leukotriene B4 (LTB4). Crude BC30 cell wall and BC supernatant were fractionated into the following molecular weight ranges: a) <3 kDa, b) 3-30 kDa, and c) 30-200 kDa, and LTB4-directed migration was examined. Similar volumes of Bacillus coagulans cell wall and supernatant were placed into centrifugation columns that filter out specific molecular weight fractions. After centrifugation, the remaining volumes were serial diluted and placed in with the PMN's before plating into the top chamber.

As described below, the repeat experiments were performed using primary immune cells from three different blood donors. In the last of these 3 experiments, an additional test was included, where crude cell wall and supernatant fractions were allowed to dry, then were reconstituted to the original sample volume. The bioactivity and dose response were tested in comparison to the non-dried cell wall and supernatant fractions in order to identify if drying and reconstitution affects biological activity in key assays.

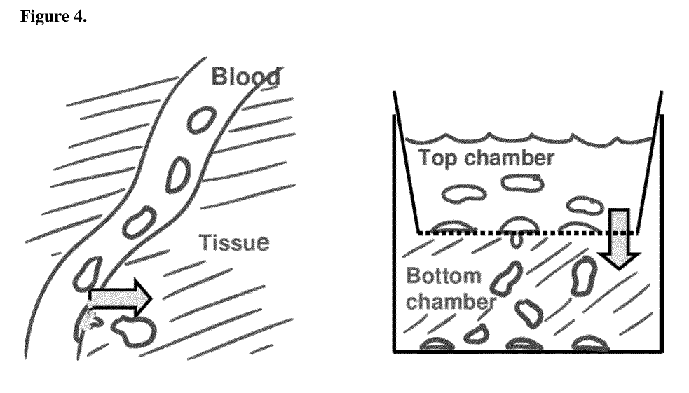

Freshly purified PMN cells cultures were set up in double-chamber migration plates, where the bottom chamber mimics tissue, and the top chamber mimics the blood stream as described in FIG. 4. Cells were plated in the top chambers with and without test products, and the different chemo-attractant (LTB4) was present in the bottom chambers. All assays were performed in quadruplicate, and repeated 3 times with cells obtained from 3 different blood donors. As described herein, the testing of migration towards the inflammatory chemo-attractant LTB4 identifies selective responses in this in vitro system, which closely mimics some in vivo models of inflammation, such as rat paw edema. The assay allows a distinction between normal PMN defense mechanisms versus response to inflammation.

### The Effect of BC Supernatant and Cell Wall Fractions on Migration

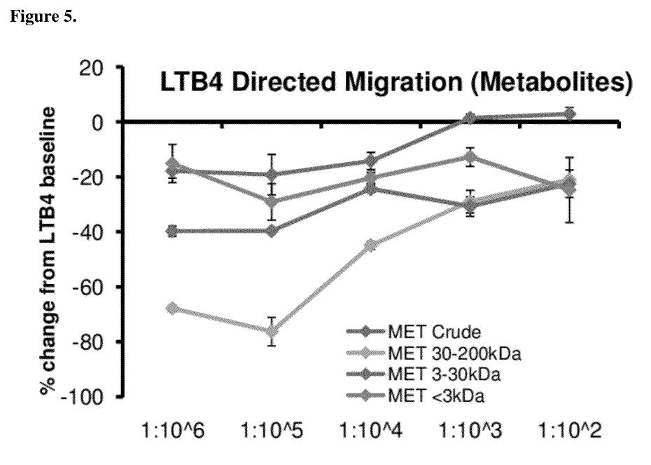

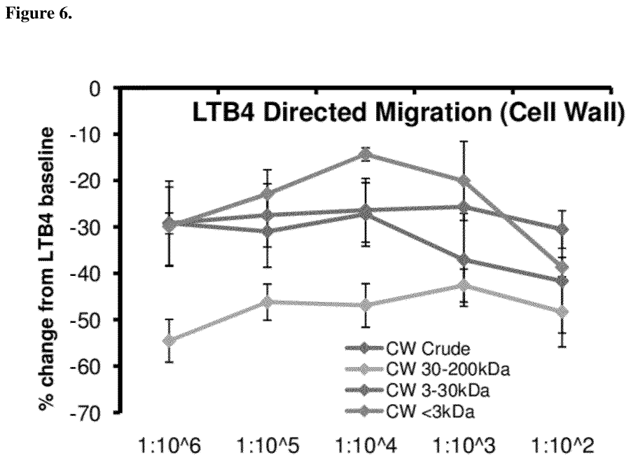

As shown in FIGS. 5 and 6, both Bacillus coagulans supernatant (metabolites) and cell wall (CW) reduced the migration of PMN cells towards the inflammatory mediator LTB4. This anti-inflammatory effect was the strongest with the 30-200 kDa fraction of both the metabolite and cell wall fractions. Because the removal of small compounds in the 30-200 kDa fraction led to an increase in reduction of migration that was greater than either crude fraction alone, it is likely that the presence of small compounds in the crude fractions inhibit the anti-inflammatory effect, and that multiple signals are generated due to the presence of differently-sized compounds in the BC30 crude fractions.

Thus, it is concluded that potent anti-inflammatory compounds exist in the high molecular weight fraction of both the supernatant (metabolites) and cell wall of Bacillus coagulans.

### The Effect of Drying/Rehydration of BC Supernatant and Cell Wall Fractions on Migration

The difference between a reconstituted sample and a normal, non-dried sample was evaluated. An aliquot of crude BC metabolites and crude BC cell wall were each allowed to evaporate under sterile conditions followed by reconstitution to their original volume with sterile water. These samples were then compared to crude metabolites and crude cell wall that had not undergone the drying and reconstitution steps.

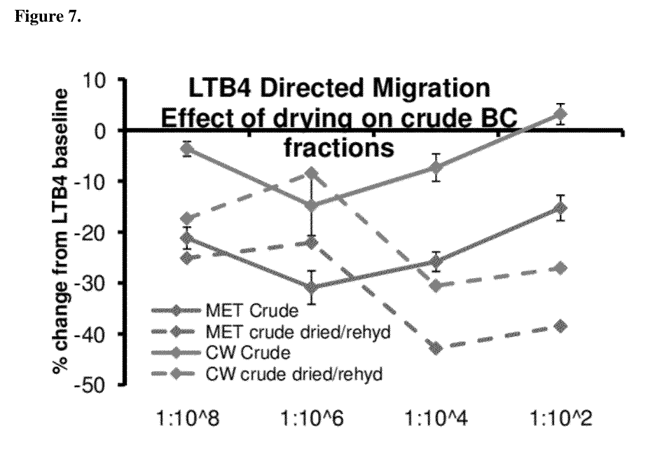

As shown in FIG. 7, the dried/reconstituted crude metabolites and crude cell wall fractions unexpectedly inhibited migration greater than the non-dried fractions. The parallel pattern of inhibition of LTB4-directed migration seen with the non-dried fractions and a different, but also parallel response seen with the dried fractions suggests that the drying and rehydration process inactivated compounds that were inhibiting the anti-inflammatory effect. Because FIGS. 5 and 6 show that the removal of the <30 kDa fraction from both the metabolite and cell wall fractions led to an enhancement in the reduction of migration of PMN cells towards LTB4, it is likely that small compounds (<30 kDa) were inactivated by the drying process.

Thus, drying and reconstitution unexpectedly increased the anti-inflammatory effect of both the supernatant (metabolites) and cell wall (CW) fractions, which was likely due to the inactivation of small compounds (<30 kDa). Moreover, the results demonstrate that the anti-inflammatory compounds present in Bacillus coagulans supernatant and cell walls are stable after drying and rehydration, which property is useful in the large scale production of these fractions.

### Example 8

### Natural Killer Cell Activation (CD69 Expression)

Crude BC30 cell wall and BC supernatant (metabolite) were fractionated into the following molecular weight ranges: a) <3 kDa, b) 3-30 kDa, and c) 30-200 kDa. Both BC fractions activated NK cells. Induction of the CD69 activation marker on the NK cells was determined in order to identify in which fractions most activity is observed.

Freshly purified human peripheral blood mononuclear cells were used for these assays. The cells are plated in 96-well micro-assay plates in duplicate. Negative control wells in quadruplicate were left untreated. Positive controls were treated with interleukin-2 (IL-2) at a dose of 100 international units per mL (IU/mL). After 18 hours of culture, cells were stained for the activation molecule CD69 on the surface of CD3-negative, CD56-positive NK cells. In this manner, the direct activation of NK cells is examined in vitro.

### The Effect of BC Supernatant and Cell Wall Fractions on NK Cell Activation

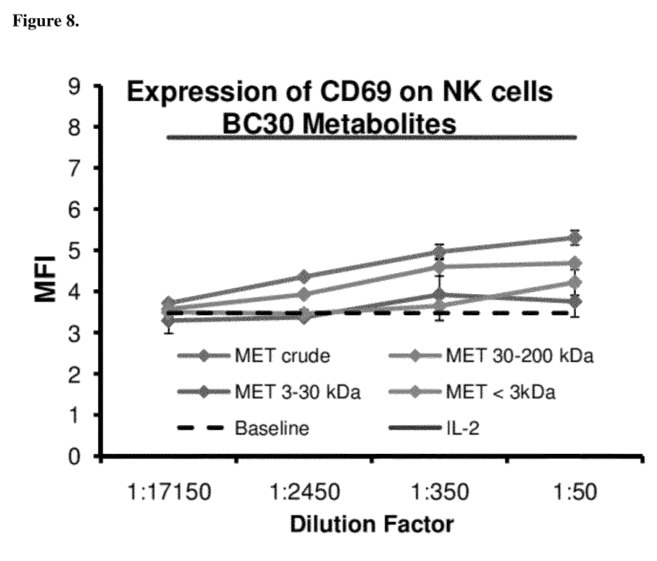

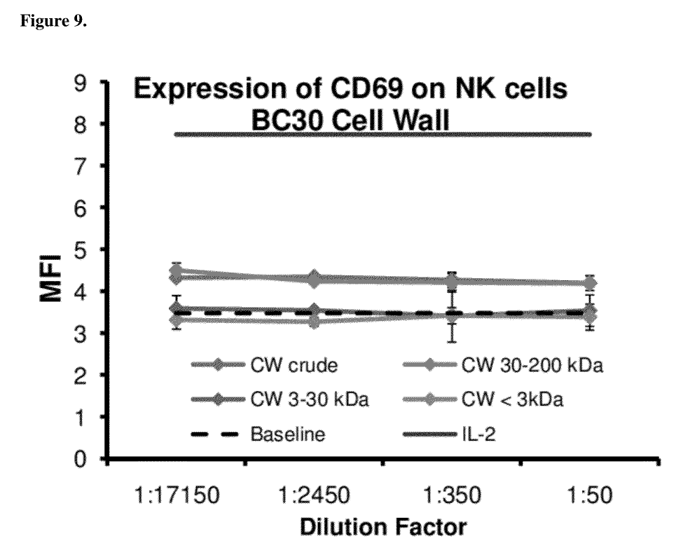

As shown in FIGS. 8 and 9, both crude supernatant (metabolites) and crude cell wall fractions increased NK cell activation as indicated by an increase in CD69 expression. However, as described below, there were some differences in supernatant and crude cell wall fractions.

Metabolites (supernatant): Crude metabolites demonstrated a dose dependent response, and the 3 highest doses resulted in highly statistically significant increases in CD69 expression (P<0.01). This increase in CD69 expression on NK cells by crude metabolites was mirrored by the 30-200 kDa fraction while the 3-30 kDa and <3 kDa fractions increased CD69 expression to much lesser degrees. Examining the effects of the individual metabolite fractions, synergy between large and small compounds both activating NK cells allows the crude preparation to work the best.

Cell Wall: Crude cell wall increased NK cell CD69 expression at all 4 doses tested (P<0.05), and an identical result was seen with the 30-200 kDa cell wall fraction. Conversely, the 3-30 kDa and <3 kDa cell wall fractions did not change NK cell CD69 expression from baseline levels. It is clear with the cell wall preparations that it is large compounds that are having an effect.

These results demonstrate that both large and small molecular weight compounds present in the metabolite preparation are capable of activating NK cells, while in the cell wall preparation, the large molecules are responsible for activating NK cells.

### The Effect of Drying/Rehydration of BC Supernatant and Cell Wall Fractions on NK Cell Activation

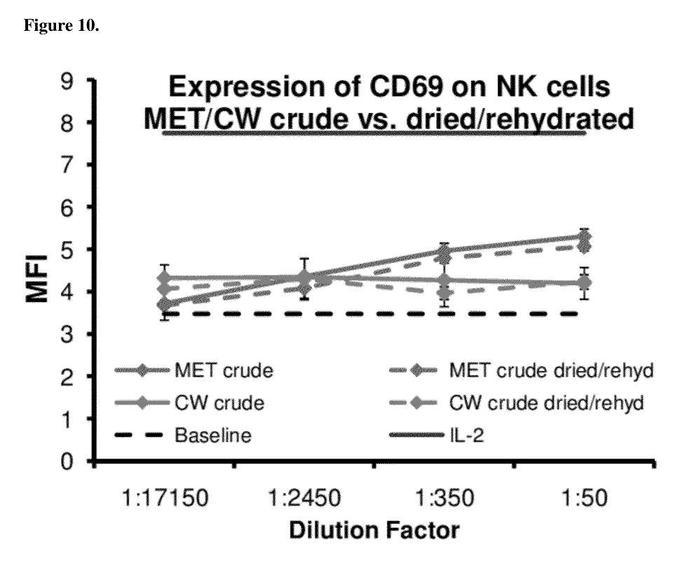

Biological activity of Bacillus coagulans supernatant and cell wall components was also assessed after drying and reconstitution to determine if bioactivity is preserved after drying. As shown in FIG. 10, drying and rehydration of crude metabolites and crude cell wall did not have much effect on the ability of the fractions to increase NK cell CD69 expression. That is, the dried and rehydrated fractions (dotted lines) parallel the activity seen with the non-dried fractions (solid lines). As shown in FIG. 10, the treatment of peripheral blood mononuclear cells (PBMC) with crude metabolites led to greater NK cell activation than treatment with crude cell wall.

### Example 9

### Lymphocyte Proliferation and Cytokine Production

Crude BC cell wall and BC supernatant were fractionated into the following molecular weight ranges: a) <3 kDa, b) 3-30 kDa, and c) 30-200 kDa. The affect of the crude and size-fractionated BC30 preparations on lymphocyte proliferation was examined.

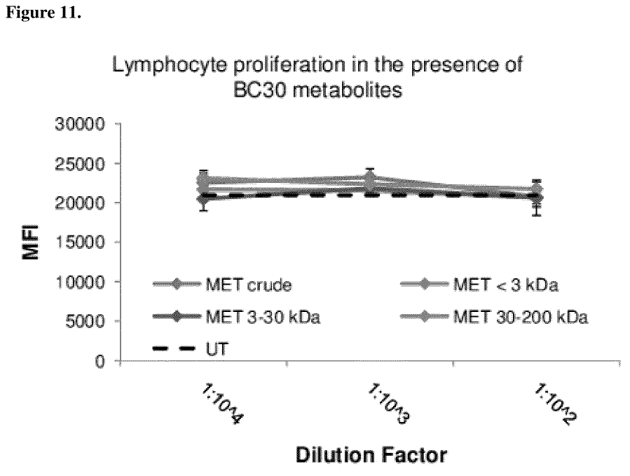

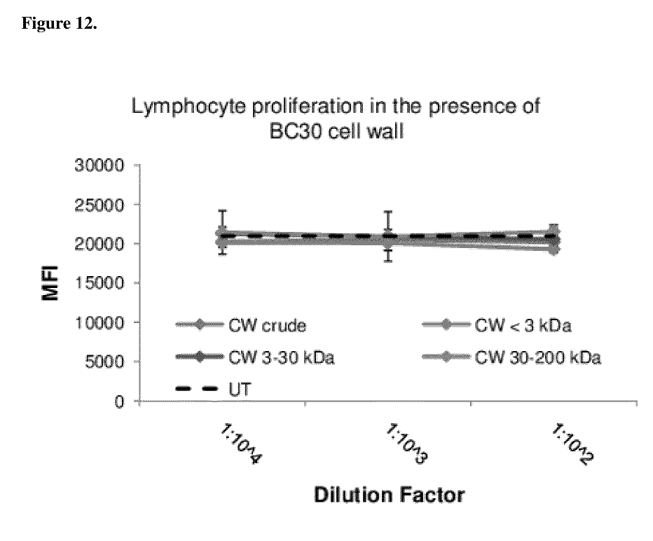

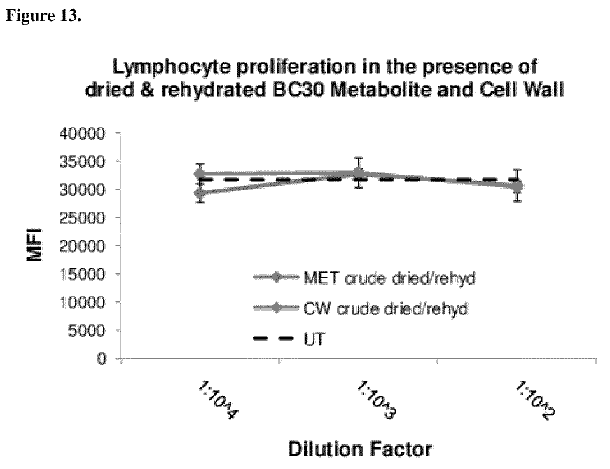

As shown in FIGS. 11-13, crude BC30 metabolites and cell wall did not affect the proliferation of PBMC in culture. The individual size-selected fractions also did not have an effect on cell proliferation. Crude metabolite and cell wall fractions that were dried and then rehydrated, performed similarly to their non-dried counterparts, i.e., there was no effect on proliferation.

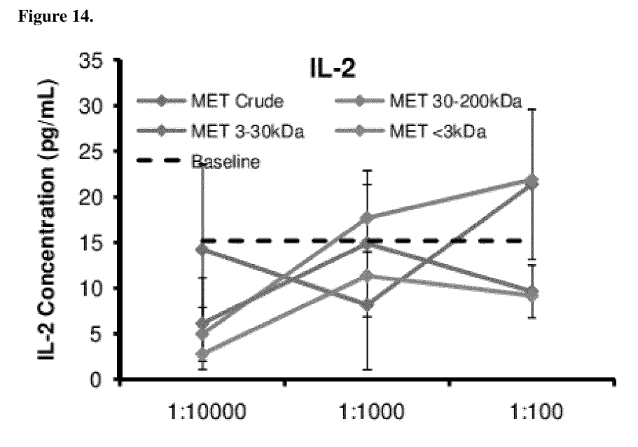

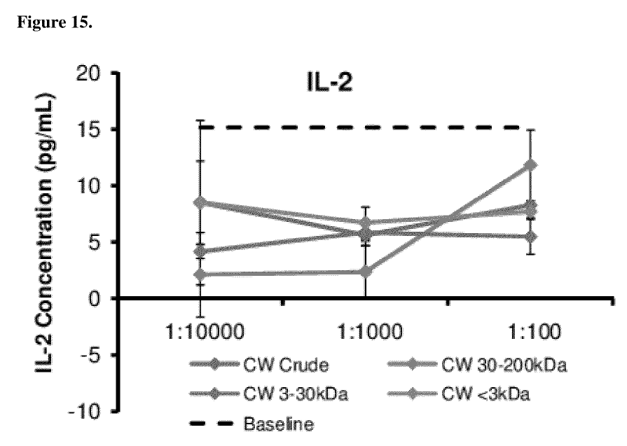

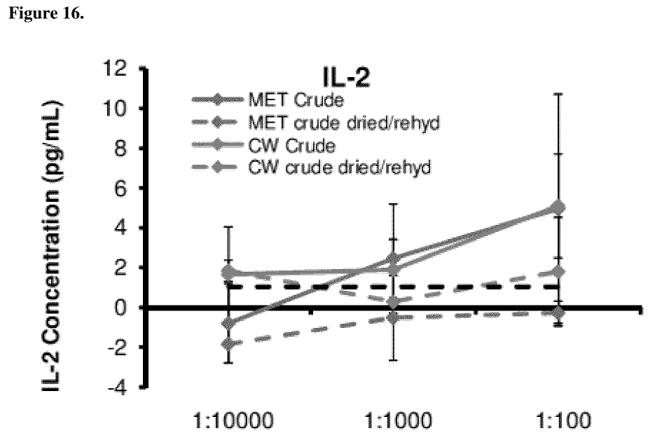

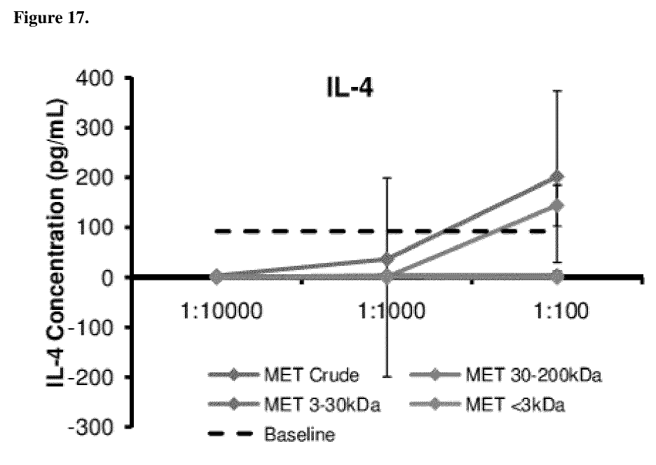

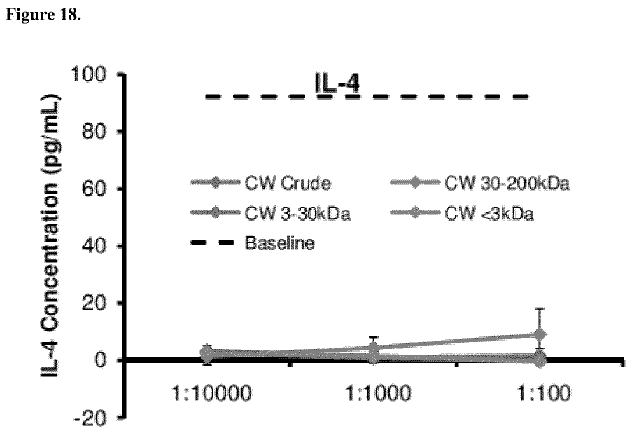

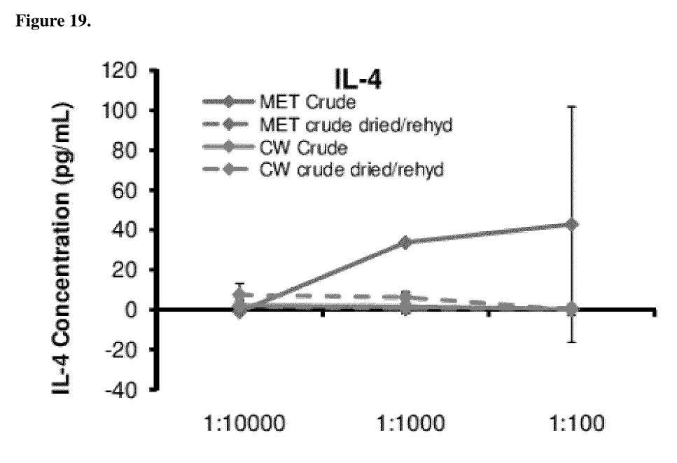

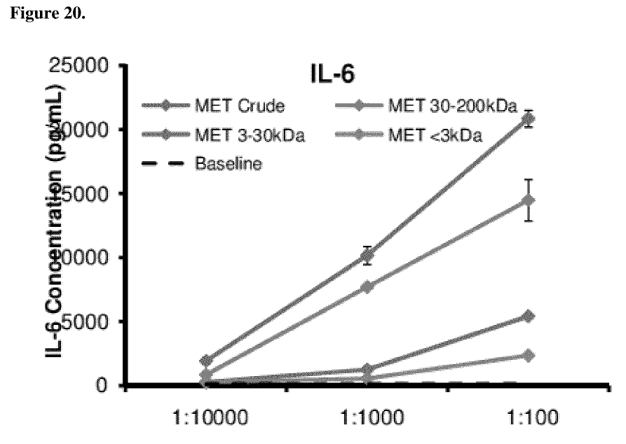

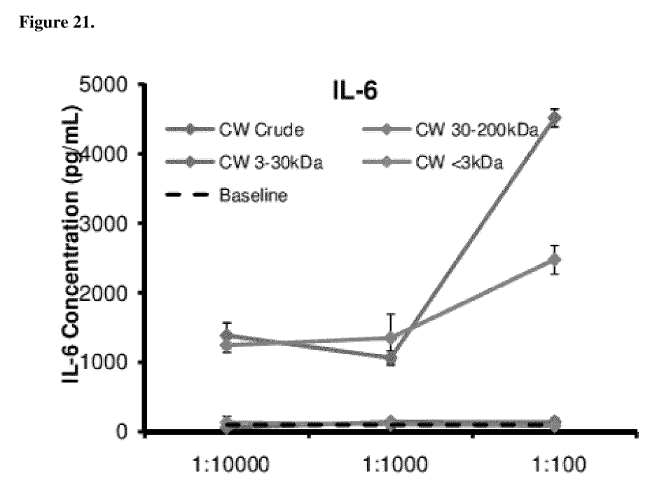

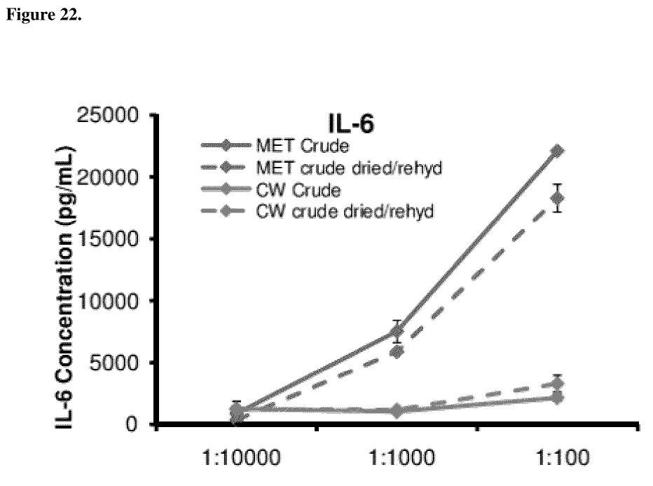

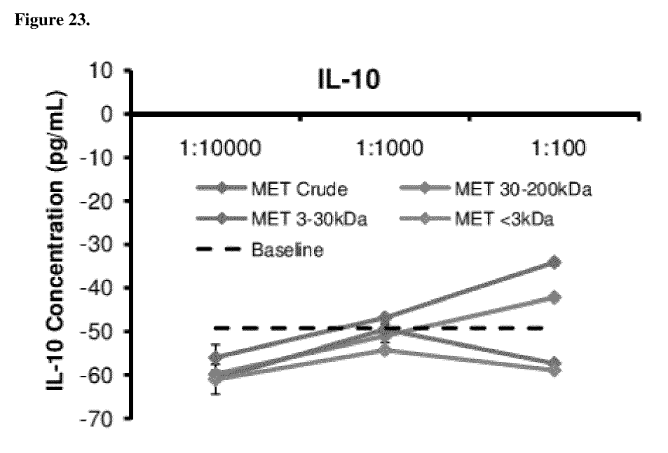

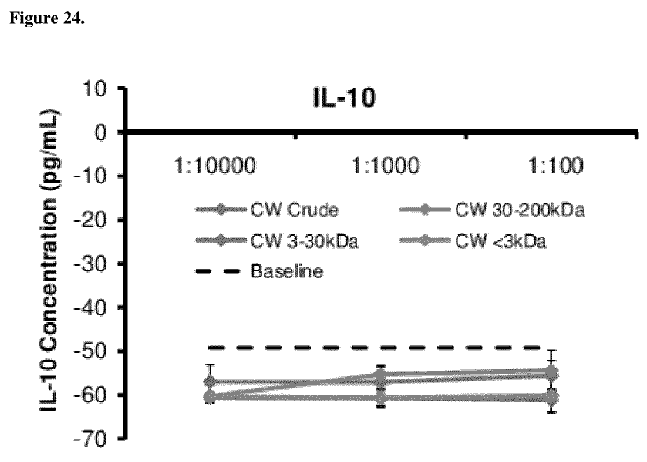

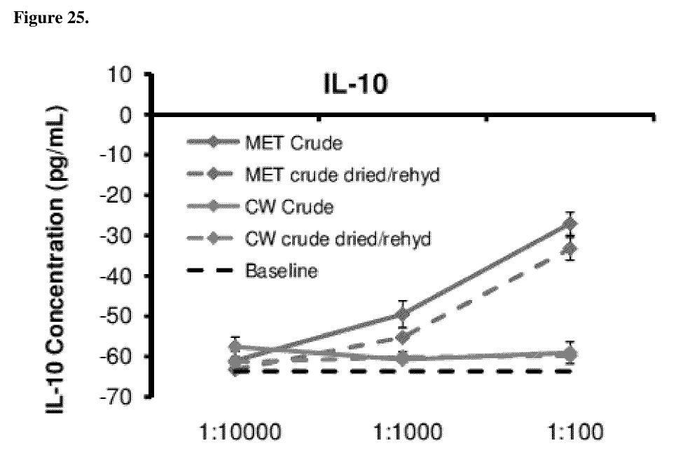

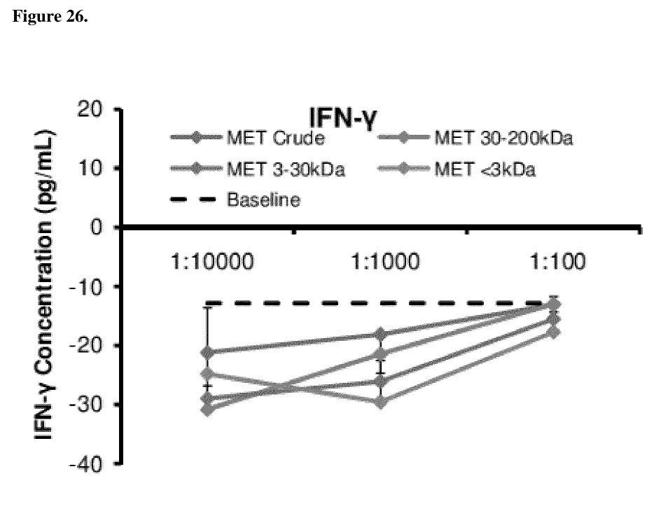

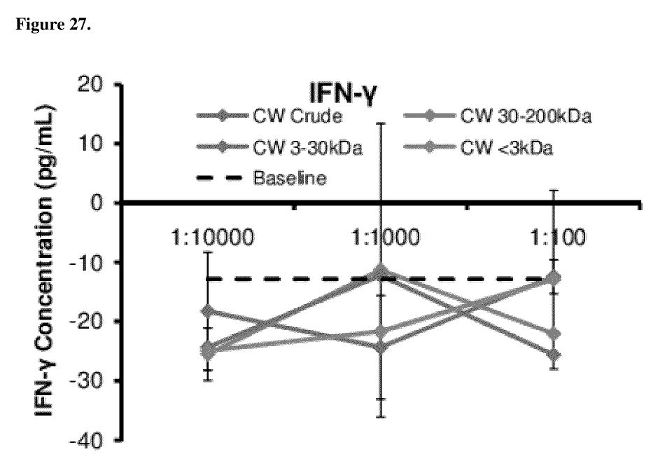

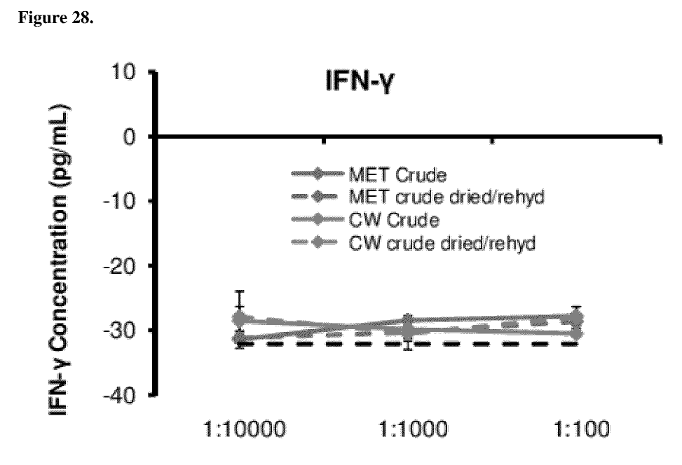

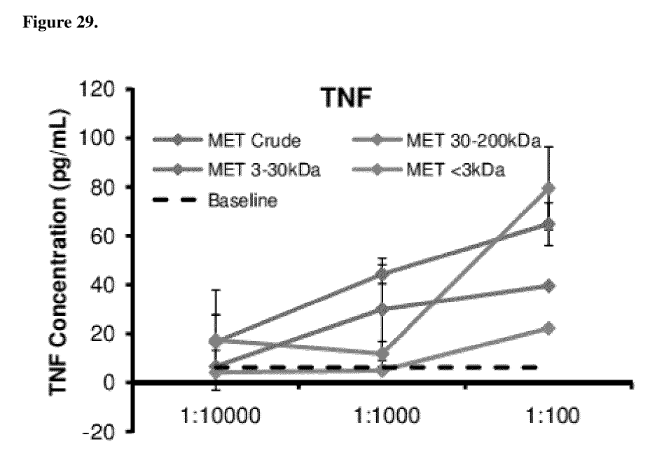

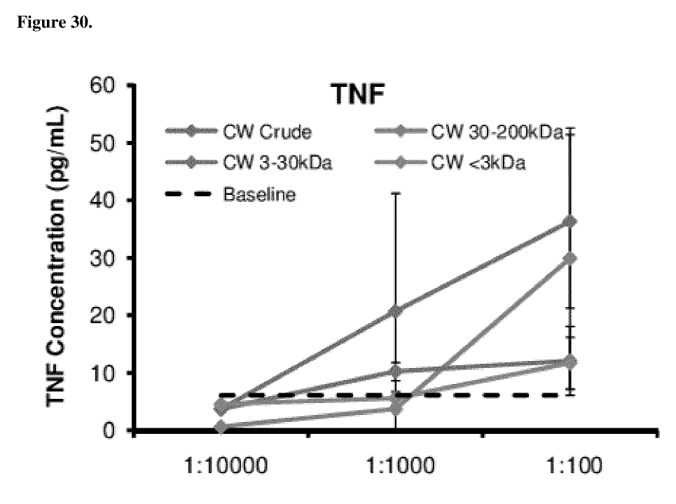

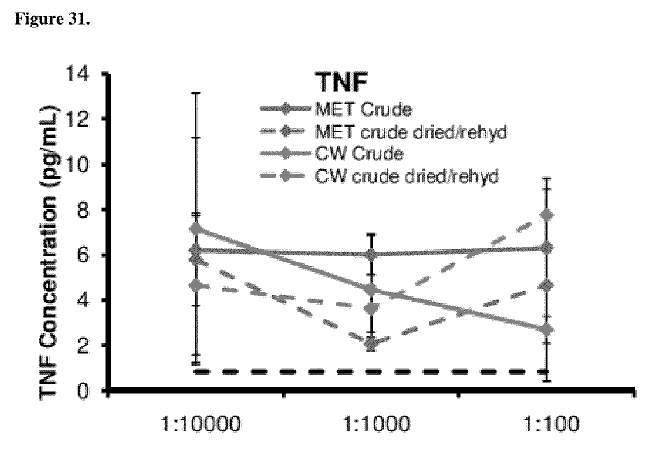

Previous experiments showed that the BC fractions directly induced changes in cytokine production. The fractions were examined to identify which molecular weight ranges of compounds in the BC supernatant and cell wall fractions are responsible for this change. Freshly purified human peripheral blood mononuclear cells (PBMC) were cultured for four days in the absence versus presence of serial dilutions of BC fractions. Supernatants from these cultures were then used to examine changes in cytokine production. Biological activity of Bacillus coagulans supernatant and cell wall components is also assessed after drying and reconstitution to determine if bioactivity is preserved after drying. The BD BioSciences cytokine bead array for flow cytometry was utilized to simultaneously measure levels of IL-2 (FIGS. 14-16), IL-4 (FIGS. 17-19), IL-6 (FIGS. 20-22), IL-10 (FIGS. 23-25), IFN-gamma (FIGS. 26-28), and TNF-alpha (FIGS. 29-31). The results shown in FIGS. 14-31 are summarized below.

Metabolites (BC30 supernatant): IL-2, IL-10 and IFN-γ did not show significant changes from untreated cells. IL-4 levels were slightly increased by the crude metabolite and 30-200 kDa metabolite fractions. IL-6 levels were highly increased by all fractions, and there was synergy of different sized molecules as indicated by the largest increase being observed with the crude fraction. TNF-alpha levels were increased by crude as well as all fractions.

Cell Wall: IL-10 and IFN-γ did not show significant changes from untreated cells. IL-2 and IL-4 levels were reduced by all cell wall fractions. IL-6 levels were greatly increased by both crude cell wall and the 30-200 kDa fraction; however, the fractions containing small compounds (<3 kDa and 3-30 kDa) had no effect. TNF-alpha levels were increased by crude cell wall as well as all fractions, but to a lesser extent than metabolites.

Thus, the results show some differences in the effects of individual fractions on cytokine production, as well as differences between the BC30 metabolites and cell wall fractions.

### Example 10

### Dendritic Cell Maturation

Probiotics and commensal bacteria interface with the mucosal immune system in the gastrointestinal tract. Dendritic cells play a major role in this interaction, and there is a direct effect of probiotics on dendritic cell biology. This includes effects on dendritic cell maturation and cytokine production.

Dendritic cells (DC) are immune cells that play an important role in both adaptive and innate immunity through their function as professional antigen presenting cells (adaptive immunity) and the generation of the type 1 interferons alpha and beta during viral infection (innate immunity). Dendritic cells circulate in the blood and are also present in environmental contact sites such as the skin and mucosal linings of the nose, lungs, stomach and intestines. Dendritic cells can be separated into different types based on expression of cell surface markers including Toll-like receptors (TLR) and by their anatomical location.

Dendritic cell maturation: Immature dendritic cells in the blood and mucosa interact with pathogens such as viruses and bacteria through toll-like receptor molecules on their surface. The recognition of antigen by immature dendritic cells results in their maturation and migration to lymph nodes where they interact with T and B cells and initiate an adaptive immune response. The maturation of dendritic cells involves the expression of a number of cell surface proteins. This maturation process can be monitored through the use of fluorescently-labeled antibodies to these cell surface proteins combined with flow cytometry. Two cell surface proteins that increase in expression during the maturation of dendritic cells are CD80 and CD86.

Dendritic cells also play a role in the development of tolerogenic/regulatory T cells that prevent the body from mounting an immune response to a particular antigen. This is an important process in the development and maintenance of immune recognition. When this recognition goes wrong, it can be seen either as wrongful self recognition such as in autoimmune disease or wrongful recognition of harmless antigens such as in allergic reactions. It may also be seen as a lack of recognition such as in immunological anergy (unresponsiveness) such as what can be involved in the development and progression of cancer.

Freshly purified human peripheral blood mononuclear cells were used for the assays described below. The cells were plated in 96-well micro-assay plates in duplicate. Negative control wells in quadruplicate were left untreated. Positive controls in triplicate were treated with LPS. Following a 3 day incubation, cells were harvested and stained with fluorescently-labeled antibodies to maturation markers. Initial staining used the combination of CD14, CD80 and CD86. The assay was repeated 3 times using cells from 3 different blood donors. These experiments on the effects of BC on dendritic cell biology was performed with BC30 crude metabolites and crude cell wall.

Part of the assessment of effects on monocytes and dendritic cells involved staining for the CD14 cell surface receptor. This is a bacterial pattern recognition receptor, also involved in recognition of lipopolysaccharide (LPS) present in the cell wall of gramnegative bacteria.

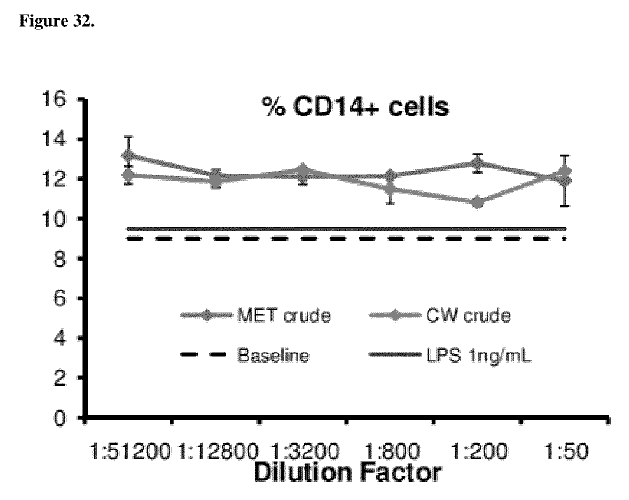

At all dilutions tested, both crude metabolites and cell wall increased the percentage of cells expressing CD14 (FIG. 32). This effect was uniform across a wide dose range. This is in contrast to LPS treatment, which did not lead to statistically significant increases in CD14 positive cells when compared to untreated cells. It is unlikely that this increase is due to proliferation of monocytes/macrophages. By contrast, it is likely that this increase is due to the differentiation of CD 14− dendritic cells towards a CD 14+ monocyte phenotype.

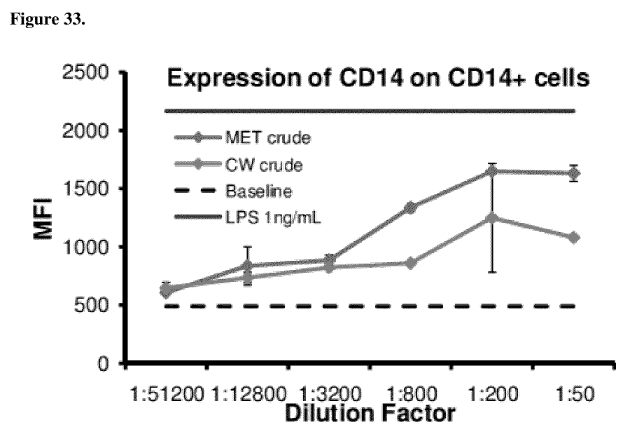

Both crude metabolites and cell wall demonstrated a dose-dependent effect on increasing the expression of CD14 on CD14+ cells (monocyte population; FIG. 33). LPS treatment led to the largest increase (almost 500%). Thus, LPS treatment induces cells that are already expressing CD14, to express more of the protein on their cell surface. The treatment of cells with the BC30 crude fractions also leads to an increase in CD14 expression on CD14+ cells, but also to a statistically significant increase in the percentage of PBMC that express CD 14. This increase in CD 14+ cells was seen even when the crude fractions were diluted 1:51,200.

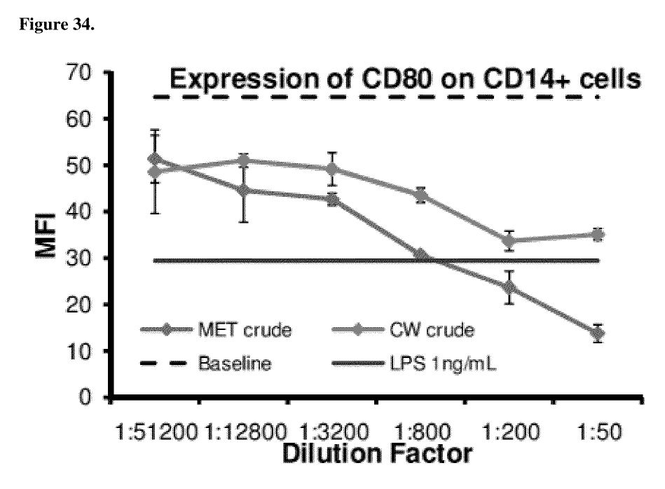

Both crude metabolite and cell wall fractions treatment of PBMC led to a decrease in CD80 expression on the cell surface of CD14+ cells (FIG. 34). At the two highest concentration of crude metabolites, CD80 expression was decreased to levels below that resulting from treatment of cells with LPS.

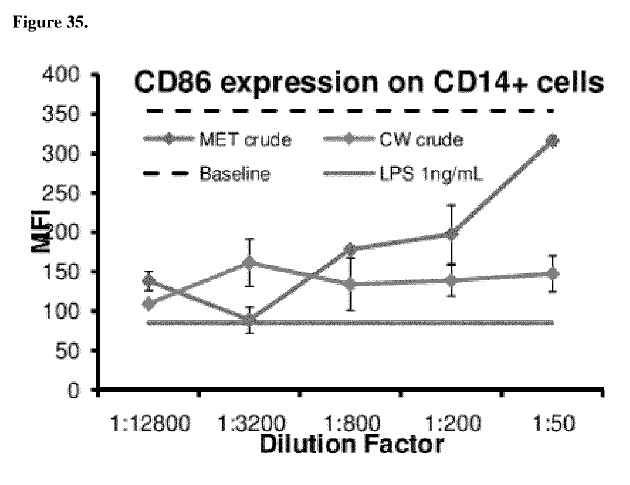

The effect of crude metabolites on CD86 expression on CD14+ cells showed an interesting dose-dependent response where the greatest reduction resulted from the lowest dose (FIG. 35). A uniform reduction in CD86 expression that was similar to that occurring with LPS treatment (1 ng/mL) resulted from treatment of PBMC with serial dilutions of the crude cell wall fraction.

The loss of CD80 and CD86 expression on CD14+ in combination with the increase in CD14+ cells indicates that dendritic cells are differentiating into a monocytoid phenotype.

### Example 11

### Evaluation of Tolerance and Efficacy of Anti-Aging Product

The purpose of this placebo-controlled study was to evaluate the performance of an anti-aging product when tested over a 4 week period in a randomized, open labeled double-blind test design. As described in detail below, wrinkle assessment was conducted instrumentally using a Visioscan image analysis system. Elasticity and viscoelastic properties of the skin were measured as a function of flexibility and firmness employing a Cutometer. Retained water content of the skin was measured using the Nova Dermal Phase Meter. Finally, each stage in the progression of treatment was photographically documented using highly developed High Resolution Scientifically Matched Photography technique. No adverse effects or unexpected reactions of any kind were observed on any of the subjects.

The test samples, i.e., active (cream with 5% Bonicel (Bacillus coagulans supernatant) or placebo (cream without Bonicel) were randomized and numbered from 1 to 10.

### Standards for Inclusion in a Study

a. Females between the ages of 35 and 60 experiencing wrinkles and lack of skin's elasticity in the face area.
b. Individuals who will complete a preliminary medical history and screening document.
c. Individuals, who will read, understand, and sign an informed consent document.
d. Individuals in general good health and free of any health problems, including neurological, dermatological, or systemic disorder that would make study participation inappropriate.
e. Individuals who will abstain from shaving or waxing the test site at least 48 hrs prior to test commencement and throughout the study.
f. Individuals able to cooperate with the Investigator and research staff, willing to have the test material(s) applied according to the protocol, and complete the full course of study.
g. Individuals with mild to moderate fine lines and wrinkles on the facial area as determined by trained technician.
h. Individuals who are currently not using any anti-aging products, and who have abstained from using them for at least 30 days prior to study commencement.
i. Individuals who are willing to abstain from use of any anti-aging products other than the assigned test article for the duration of the study.
Standards for Exclusion from a Study
a. Individuals who are under the care of a physician being treated for specific condition that may interfere with the study design.
b. Individuals currently taking medication that may mask or interfere with the test results.
c. Individuals diagnosed with chronic skin allergies.
d. Females who are pregnant, lactating, have been pregnant, or given birth within the six month period immediately preceding study commencement.
e. Subjects with a history of any form of skin cancer, melanoma, lupus, psoriasis, connective tissue disease, diabetes, or any disease that would increase the risk associated with study participation.
f. Individuals who have experienced irritation or sensitivity to lotion products.
g. Individuals with known allergies or skin and/or eye conditions, which would interfere with the study.

### Participant (Panel) Demographics

Number of subjects enrolled: 10
Number of subjects completing study: 10

### Age Range: 43-54

### Sex Female: 10

### Race Caucasian: 10

### Procedure

Ten healthy females were inducted into this study. The samples—Active and Placebo—were randomized and numbered (from 1 to 10). All test products appeared identical to placebo to protect the study blind. At the completion of the study, upon the receipt of the data, the decoding table of random sampling numbers (including the sample description) was used for the purpose of statistical analysis and data reporting.

As a condition of enrollment, only the subjects who were currently not using any anti-aging products, and who have abstained from using them for at least 30 days prior to study commencement were recruited for participation in this investigation. On the initial day of the study, upon arrival at the testing facility, subjects were required to familiarize themselves with and sign the informed consent. Subjects were mandated to adhere to all the restrictions mentioned in the inclusion/exclusion section. All participants were advised of the general nature and purpose of this study. The subjects then acclimated to the ambient environment for a period of thirty minutes prior to baseline evaluation. The same acclimation procedure was applied to any following evaluation time point.

All 10 participants of the study received the test product. Neither the investigator nor the test subjects were aware if they received an active product or placebo. Prior to baseline measurements were taken, areas of involvement were marked on the facial surface using a standard template, to ensure that instruments were repositioned in the same place at each visit. As described in detail below, all biophysical measurements (Skin Moisturization—Electroconductivity via Novameter, Surface Evaluation of Living Skin via Visioscan and Skin Elasticity via Cutometer) were conducted by a trained technician, and Pre-treatment High Resolution Scientifically Matched Photographs were taken.

Panelists received verbal and written instructions regarding product use and study restrictions. Subjects were required to use the test product as a part of their daily routine according to the following instructions: “Use twice daily. On a clean, dry skin, apply cream to forehead, eye area and cheeks. Rub product in completely as appropriate. Apply usual make-up as needed.”

After 14 and 28 days of daily use of the test product, test subjects were re-evaluated. After acclimating to ambient conditions, the measurements (Skin Moisturization—Electroconductivity via Novameter, Surface Evaluation of Living Skin via Visioscan and Skin Elasticity via Cutometer) were repeated using the standard template to identify sites on the face and, High Resolution Scientifically Matched Photographs were taken. Specifically, the following distinct noninvasive methods were employed to establish evaluation parameters.

### Electroconductivity—Skin Moisturization—Nova Dermal Phase Meter (“Novameter”)

A Nova Dermal Phase Meter, Model DPM 9003 (Nova Technology Corp., Gloucester, Mass.) was used to obtain measurements of skin surface impedance to determine electroconductivity of the treatment sites. The DPM 9003 is a portable, multifunctional electronic laboratory instrument that measures skin impedance, and was designed to provide a non-invasive, objective, reproducible method of measurement to quantify biophysical characteristics and relative hydration of the skin. This meter provides a relative measure of the retained water content of the skin as a function of the skin's dielectric value. The Nova Dermal Phase Meter (DPM) is used in the art as an impedance-based instrument using capacitive re-actance values expressed in arbitrary DPM units.

Specifically, as described in Clarys et al., 1999 Skin Research and Technology, 5: 14-20 (“Clarys,” incorporated herein by reference), the Nova DPM 9003 (Nova Technology Corporation) measures impedance based capacitive reactance of the skin at preselected frequencies up to 1 MHz from the observed signal phase delays. The standard 8 mm probe features (0.9 cm² surface) two concentric brass ring electrodes separated by an isolator (with respective inner and outer diameter of 4.34 and 8.76 mm). The distance between the inner and the outer electrode is 1 mm. There is direct galvanic contact between the electrodes and the skin. By integrating measurements at the preselected frequencies, capacitive reactance is calculated from the signal and phase delay using an integrated circuit in the instrument. The final readout is given in arbitrary DPM units, ranging from 90 to 999 DPM units, which are directly related to the capacitance. An automatic calibration takes place, ensuring standardization of the instrument before taking any readings.

Clarys also describes other instruments used in dermato-cosmetic research, including the Corneometer CM 825 (Courage+Khazaka Electronic GmbH, Köln, Germany) and the Skicon-200 (ISBS Co, Hamamatsu, Japan).

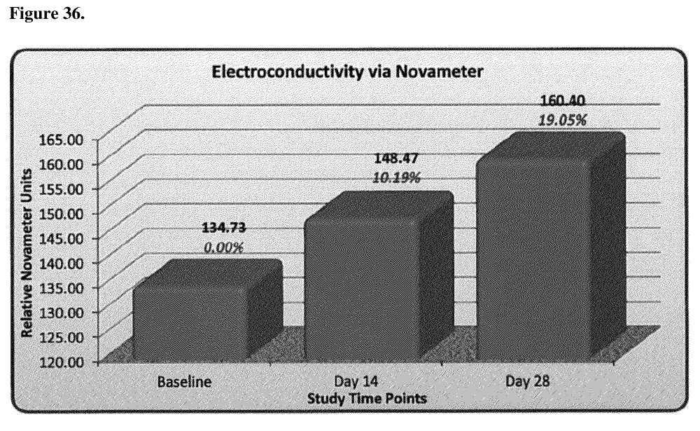

Skin impedance was recorded automatically when equilibrium was achieved. See, Leveque and de Rigal, 1983 J. Soc. Cosmet. Chem., 34: 419-428, incorporated herein by reference. As shown in Table 43 below, Novameter readings demonstrated that the test product M-7293 (i.e., cream with 5% Bonicel (Bacillus coagulans supernatant) dramatically increased the skin moisture content. The increases are considered statistically significant after 14 and 28 days of use (FIG. 36).

TABLE 43
Electroconductivity via Novameter - Skin Moisturization
AMA Lab No.: M-7293
Client No.: Cream with Bonicel (bacillus ferment), Lot 28378

| | | | Indi- | | Idi- |
|---|---|---|---|---|---|
| Panelist ID | Base- | | vidual % | | vidual % |
| No.: | line | Day 14 | Difference | Day 28 | Difference |
| 56 8611 | 157.33 | 173.67 | 10.39% | 179.67 | 14.20% |
| 56 0637 | 146.00 | 167.67 | 14.84% | 190.00 | 30.14% |
| 66 0675 | 152.67 | 172.00 | 12.66% | 184.33 | 20.74% |
| 48 2833 | 115.67 | 117.67 | 1.73% | 133.67 | 15.56% |
| 50 0190 | 102.00 | 111.33 | 9.15% | 114.33 | 12.09% |
| Mean: | 134.73 | 148.47 | | 160.40 | |

| | | |
|---|---|---|
| % Difference | 10.19% | 19.05% |
| p | 0.019* | 0.010* |
| t | 3.823* | 10.516* |

*Statistically Significant

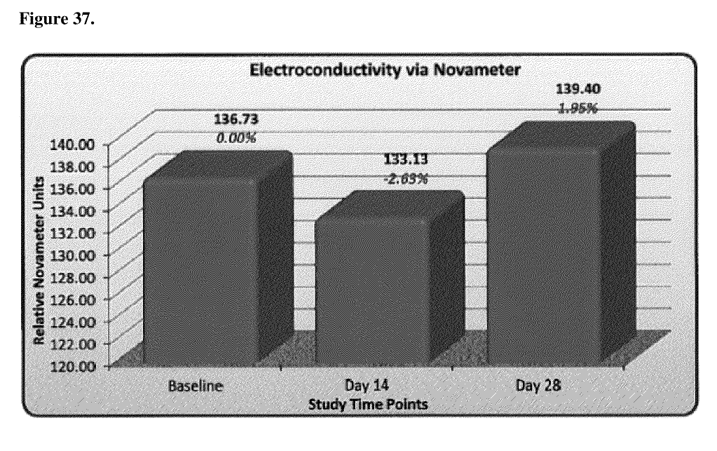

As shown in Table 44 below, Novameter readings demonstrated that the test product M-7294 (i.e., cream without Bonicel (Bacillus coagulans supernatant) did not increase the skin moisture content (FIG. 37).

TABLE 44
Electroconductivity via Novameter - Skin Moisturization
AMA Lab. No.: M-7294
Client No.: Cream without Bonicel, Lot 28378

| | | | Indi- | | Indi- |
|---|---|---|---|---|---|
| Panelist ID | Base- | | vidual % | | vidual % |
| No.: | line | Day 14 | Difference | Day 28 | Difference |
| 56 0900 | 125.33 | 114.33 | −8.78% | 131.33 | 4.79% |
| 52 3397 | 159.00 | 143.33 | −9.86% | 145.67 | −8.38% |
| 62 9653 | 128.67 | 134.00 | 4.14% | 129.00 | 0.26% |
| 58 6382 | 122.67 | 126.33 | 2.98% | 139.67 | 13.86% |
| 50 7599 | 148.00 | 147.67 | −0.22% | 151.33 | 2.25% |
| Mean: | 136.73 | 133.13 | | 139.40 | |

| | | |
|---|---|---|
| % Difference | −2.63% | 1.95% |
| p | 0.434 | 0.615 |
| t | 0.869 | 33.059 |

* Statistically Significant

A summary of the Novameter readings for each of the two groups (i.e., with and without Bonicel) is provided in Table 45 below.

TABLE 45
Electroconductivity via Novameter - Skin Moisturization

| | |
|---|---|
| Day 14 | Day 28 |

Active Treatment Group
AMA Lab No.: M-7293
Client No.: Cream with Bonicel (bacillus ferment), Lot 28378

| | | |
|---|---|---|
| % Difference: | 10.19%* | 19.05%* |
| Max % Improvement: | 14.84% | 30.14% |

Placebo Group
AMA Lab No.: M-7294
Client No.: Cream without Bonicel, Lot 28378

| | | |
|---|---|---|
| % Difference: | −2.63% | 1.95% |
| Max % Improvement: | 4.14% | 13.86% |

*Statistically Significant

Surface Evaluation of Living Skin—Visioscan

The Visioscan, e.g., Visioscan® VC 98, (Courage+Khazaka Electronic GmbH, Köln, Germany) takes a direct image of the living skin using a measuring head containing a CCD-camera featuring a high resolution video sensor and two metal halogen lamps positioned opposite each other in order to ensure even/uniform illumination of the measuring field on the skin. The resulting images are displayed in 256 gray levels. The grey level distribution of the pixels in the image correspond to different phenomena (white pixels represent desquamation/scaliness on the skin, dark pixels represent lines and wrinkles). The software analyzes the gray level distribution and calculates four clinical parameters to quantitatively and qualitatively describe the skin surface as an index: skin smoothness, skin roughness, scaliness and wrinkles. See, Fischer et al., 1999 Skin Pharmacol Appl Skin Physiol, 12: 1-11; Farwick et al., 2009 An EC-derived Tetrapeptide to Counterbalance ECM Degeneration; Cosmetic & Toiletries magazine, Vol 124 Np. 6/June, each of which is incorporated herein by reference.

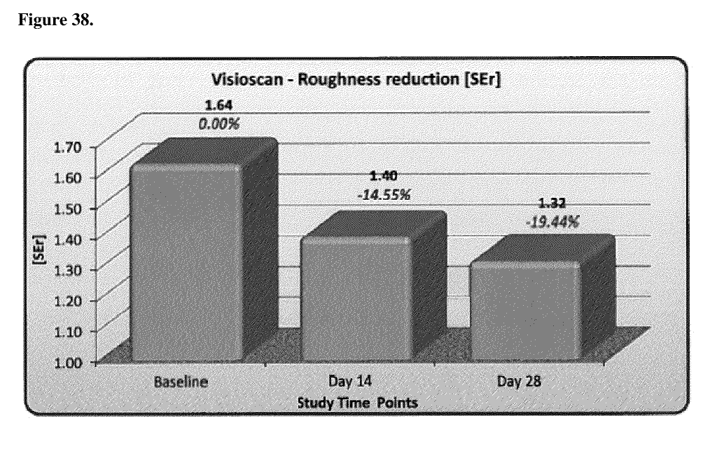

As shown in Table 46 below, within the limits imposed by the conduct and population size of the placebo-controlled study, the anti-aging test material (AMA Lab No.: M-7293 (Cream with 5% Bonicel (Bacillus coagulans supernatant), Lot 28378) demonstrated a dramatic decrease compared to placebo treatment (AMA Lab No.: M-7294 (Cream without Bonicel, Lot 28378) in the Visioscan parameters of surface roughness (SEr) associated with the depth of fine and course wrinkles. The reductions were considered statistically significant after 28 days of use (FIG. 38).

TABLE 46
Visioscan - Roughness reduction [SEr]
AMA Lab No.: M-7293
Client No.: Cream with Bonicel (bacillus ferment), Lot 28378

| | | | Indi- | | Indi- |
|---|---|---|---|---|---|
| Panelist ID | Base- | | vidual % | | vidual % |
| No.: | line | Day 14 | Difference | Day 28 | Difference |
| 56 8611 | 2.11 | 1.80 | −0.15 | 1.79 | −15.17% |
| 56 0637 | 1.03 | 1.07 | 3.88% | 0.94 | −8.74% |
| 66 0675 | 1.97 | 1.88 | −4.57% | 1.67 | −15.23% |
| 48 2833 | 1.59 | 1.26 | −20.75% | 1.23 | −22.64% |
| 50 0190 | 1.48 | 0.98 | −33.78% | 0.96 | −35.14% |
| Mean: | 1.64 | 1.40 | | 1.32 | |

| | | |
|---|---|---|
| % Difference | −14.55% | −19.44% |
| p | 0.067 | 0.010* |
| t | 2.498 | 8.202* |

*Statistically Significant

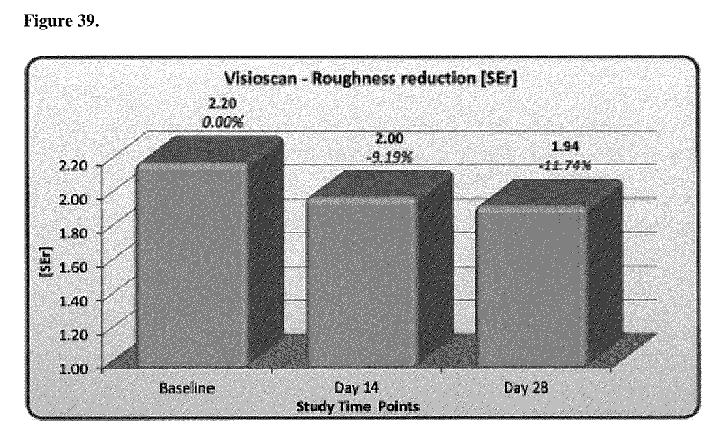

As shown in Table 47 below, Visioscan readings demonstrated that the test product M-7294 (i.e., cream without Bonicel (Bacillus coagulans supernatant) did not decrease surface roughness associated with the depth of fine and course wrinkles (FIG. 39).

TABLE 47
Visioscan - Roughness reduction [SEr]
AMA Lab No.: M-7294
Client No.: Cream without Bonicel, Lot 28378

| | | | Indi- | | Indi- |
|---|---|---|---|---|---|
| Panelist ID | Base- | | vidual % | | vidual % |
| No.: | line | Day 14 | Difference | Day 28 | Difference |
| 56 0900 | 1.78 | 1.48 | −16.85% | 1.37 | −23.03% |
| 52 3397 | 2.24 | 2.36 | 5.36% | 2.38 | 6.25% |
| 62 9653 | 2.65 | 2.16 | −18.49% | 2.12 | −20.00% |
| 58 6382 | 1.41 | 1.29 | −8.51% | 1.12 | −20.57% |
| 50 7599 | 2.91 | 2.69 | −7.56% | 2.71 | −6.87% |
| Mean: | 2.20 | 2.00 | | 1.94 | |

| | | |
|---|---|---|
| % Difference | −9.19% | −11.74% |
| p | 0.116 | 0.086 |
| t | 2.004 | 7.061 |

* Statistically Significant

A summary of the Visioscan readings for each of the two groups (i.e., with and without Bonicel) is provided in Table 48 below.

TABLE 48
Visioscan - Roughness reduction [SEr]

| | |
|---|---|
| Day 14 | Day 28 |

Active Treatment Group
AMA Lab No.: M-7293
Client No.: Cream with Bonicel (bacillus ferment), Lot 28374

| | | |
|---|---|---|
| % Difference: | −14.55% | −19.44%* |
| Max % Improvement: | −33.78% | −35.14 |

Placebo Group
AMA Lab No.: M-7294
Client No.: Cream without Bonicel, Lot 28378

| | | |
|---|---|---|
| % Difference: | −9.19% | −11.74% |
| Max % Improvement: | −18.49% | −23.03% |

*Statistically Significant

### Skin Elasticisty—Cutometer

A Cutometer SEM 575 (Courage+Khazaka Electronic GmbH, Köln, Germany) was used to measure skin viscoelastic properties. The Cutometer dual MPA 580 (Courage+Khazaka Electronic GmbH, Köln, Germany) is also a suitable tool to measure skin viscoelastic properties. The measuring principle is based on a suction method. Negative pressure is created in the device, which can be regulated between 20 and 500 mbar. Skin is drawn into a calibrated aperture of the probe by negative pressure and after a defined time, released again. Inside the probe, the skin penetration depth is determined by a non-contact optical measuring system. The optical measuring system consists of a light transmitter and a light recipient, as well as two glass prisms facing each other, which project the light from transmitter to recipient. The light intensity will vary due to the penetration depth of the skin. The resistance of the skin to the negative pressure (firmness) and its ability to return into its original position (elasticity) are displayed as curves (penetration depth in mm/time) in real time during the measurement. This measurement principle allows for obtaining information about the elastic and mechanical properties of skin surface and enables the objective quantification of skin aging. Well-established elasticity parameters, including firmness, resistance to suction, and fatigue can be determined. See, Agache et al., 1980 Arch. Dermatol. Res., 269: 221; de Rigal and Leveque et al., 1985 Bioeng. Skin, 1: 13, each of which is incorporated herein by reference.

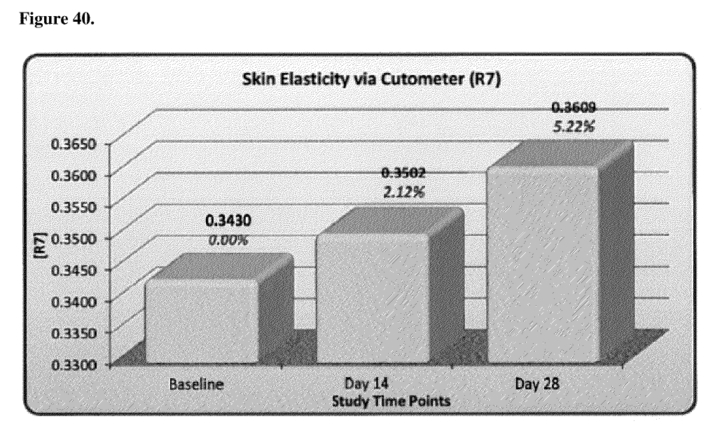

As shown in Table 49 below, evaluation of the skin's Elasticity/Flexibility via Cutometer demonstrated an increase in biological elasticity in the group treated with the test product M-7293 (i.e., cream with 5% Bonicel (Bacillus coagulans supernatant)) after 14 and 28 days (FIG. 40).

TABLE 49
Skin Elasticity via Cutometer (R7)
AMA Lab No.: M-7293
Client No.: Cream with Bonicel (bacillus ferment), Lot 28378

| | | | Indi- | | Indi- |
|---|---|---|---|---|---|
| Panelist ID | Base- | | vidual % | | vidual % |
| No.: | line | Day 14 | Difference | Day 28 | Difference |
| 56 8611 | 0.3211 | 0.3106 | −3.27% | 0.3271 | 1.87% |
| 56 0637 | 0.3357 | 0.3493 | 4.05% | 0.3512 | 4.62% |
| 66 0675 | 0.4276 | 0.4669 | 9.19% | 0.4835 | 13.07% |
| 48 2833 | 0.2804 | 0.2699 | −3.74% | 0.2772 | −1.14% |
| 50 0190 | 0.3501 | 0.3545 | 1.26% | 0.3655 | 4.40% |
| Mean: | 0.3430 | 0.3502 | | 0.3609 | |

| | | |
|---|---|---|
| % Difference | 2.12% | 5.22% |
| p | 0.476 | 0.151 |
| t | 0.786 | 22.942 |

*Statistically Significant

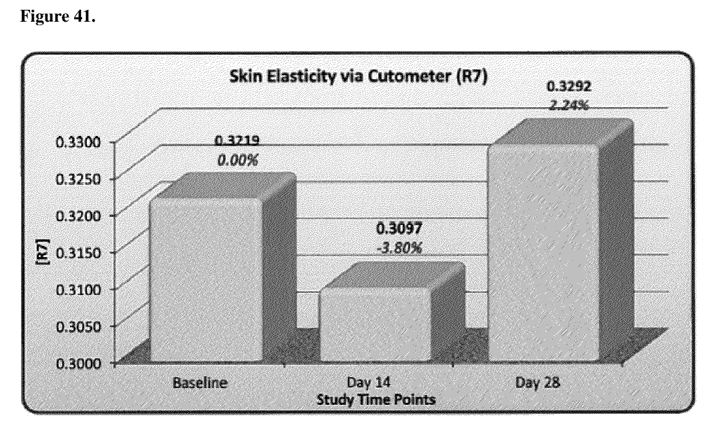

As shown in Table 50 below, evaluation of the skin's Elasticity/Flexibility via Cutometer did not demonstrate an increase in biological elasticity in the group treated with the test product M-7294 (i.e., cream without Bonicel (Bacillus coagulans supernatant) (FIG. 41).

TABLE 50
Skin Elasticity via Cutometer (R7)
AMA Lab No.: M-7294
Client No.: Cream without Bonicel, Lot 28378

| | | | Indi- | | Indi- |
|---|---|---|---|---|---|
| Panelist ID | Base- | | vidual % | | vidual % |
| No.: | line | Day 14 | Difference | Day 28 | Difference |
| 56 0900 | 0.3862 | 0.3423 | −11.37% | 0.3826 | −0.93% |
| 52 3397 | 0.3027 | 0.2576 | −14.90% | 0.2811 | −7.14% |
| 62 9653 | 0.2978 | 0.3340 | 12.16% | 0.3358 | 12.76% |
| 58 6382 | 0.3010 | 0.3012 | 0.07% | 0.3342 | 11.03% |
| 50 7599 | 0.3220 | 0.3135 | −2.64% | 0.3121 | −3.07 |
| Mean: | 0.3219 | 0.3097 | | 0.3292 | |

| | | |
|---|---|---|
| % Difference | −3.80% | 2.24% |
| p | 0.465 | 0.579 |
| t | 0.806 | 7.571 |

* Statistically Significant

A summary of the skin elasticity readings for each of the two groups (i.e., with and without Bonicel) is provided in Table 51 below.

TABLE 51
Skin Elasticity via Cutometer (R7)

| | |
|---|---|
| Day 14 | Day 28 |

Active Treatment Group
AMA Lab No.: M-7293
Client No.: Cream with Bonicel (bacillus ferment), Lot 28378

| | | |
|---|---|---|
| % Difference: | 2.12% | 5.22% |
| Max % Improvement: | 9.19% | 13.07% |

Placebo Group
AMA Lab No.: M-7294
Client No.: Cream without Bonicel, Lot 28378

| | | |
|---|---|---|
| % Difference: | −3.80% | 2.24% |
| Max % Improvement: | 12.16% | 12.76% |

* Statistically Significant

### Reverse Photo Engineering

Exclusively detailed, high resolution before and after digital photographs were taken, with fixed camera background, distances, angles, settings, lighting, panelist positioning, color bars, white balance, standardized and digitally certified unretouched (AMA Laboratories, Inc., NY, N.Y.). Each stage in the progression of the treatment regimen was photographically documented and the test area of involvement isolated. Photographs were evaluated using image analysis software which allows the evaluation parameter (e.g., wrinkles) to be captured and quantified. Image analysis software detects subtle changes in color by three dimensional profile of hue, value and chroma. These characteristics are then translated into color coordinates (a*, b* and L*) whose spacing is considered with the color changes perceived by the human eye. The size of the area of involvement differs for each test panelist. Therefore, percent difference is calculated individually and then averaged. [px²]-wrinkle related pixels per area of involvement. Suitable dermatological image software analysis programs include, e.g., Mirror™ software (Canfield Scientific Inc. Fairfield, N.J.), 3D LifeViz™ (Quantificare Inc., San Mateo, Calif.), and Sculptor 3D simulation (Canfield Scientific Inc. Fairfield, N.J.).

Student's t-test was used for statistical analysis. This is the test of the null hypothesis that the difference between two responses measured on the same statistical unit has a mean value of zero. The changes in wrinkle size (area affected by wrinkle measured in px²) before and after the treatment were measured. If the treatment is effective, the area affected by wrinkle for the subjects is smaller following the treatment. This is often referred to as the “paired” or “repeated measures” t-test. Dependent samples (or “paired”) t-tests typically consist of a sample of matched pairs of similar units, or one group of units that has been tested twice (a “repeated measures” t-test).

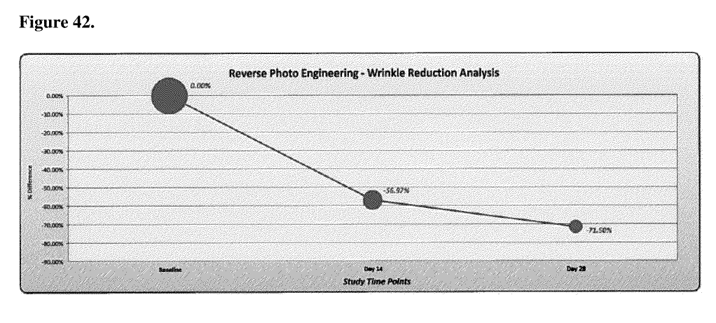

As shown in Table 52 below, data obtained through image analysis software demonstrated wrinkle reduction after 14 and 28 days of usage of the test product AMA Lab No.: M-7293 (i.e., cream with 5% Bonicel (Bacillus coagulans supernatant). Each stage in the progression of the treatment regimen was photographically documented, and the test area of involvement was isolated. Photographs were evaluated using image analysis software which allows the wrinkles to be captured and quantified. The size of the area of Involvement differed for each test subject. Therefore, the percent difference was calculated individually and then averaged. [px²]—wrinkle related pixels per area of involvement. The results are considered statistically significant (FIG. 42).

TABLE 52
Reverse Photo Engineering - Wrinkle and Fine Lines Reduction Analysis
AMA Lab No.: M-7293
Client No.: Cream with Bonicel (bacillus ferment), Lot 28378

| Panelist ID Nos. | Baseline (px) | Day 14 (px) | Individual % Difference | Day 28 (px) | Individual % Difference |
|---|---|---|---|---|---|
| 56 8611 | 2103 | 811 | −61.44% | 516 | −75.46% |
| 56 0637 | 6001 | 3179 | −47.03% | 2570 | −57.17% |
| 66 0675 | 11115 | 3278 | −70.51% | 1840 | −83.45% |
| 48 2833 | 9948 | 6159 | −38.09% | 4541 | −54.35% |
| 50 0190 | 6489 | 2089 | −67.81% | 840 | −87.06% |

| | | |
|---|---|---|
| Average % Difference: | −56.97% | −71.50% |
| Max % Reduction: | −70.51% | −87.06% |
| p | 0.021* | 0.07* |
| t | 3.705* | 3.950* |

*Statistically Significant

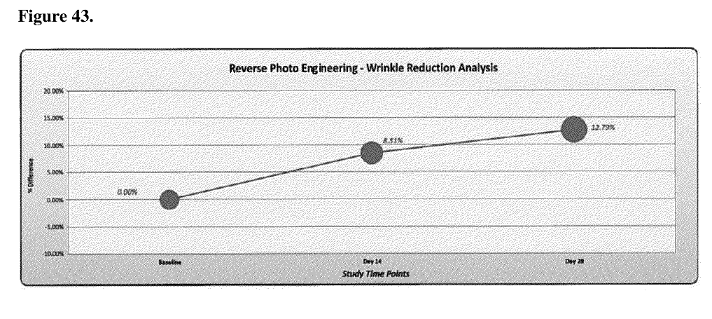

As shown in Table 53 below, data obtained through image analysis software demonstrated no improvement in wrinkle reduction after 14 and 28 days of usage of the test product AMA Lab No.: M-7294 (Cream without Bonicel, Lot 28378; FIG. 43).

TABLE 53
Reverse Photo Engineering - Wrinkle and Fine Lines Reduction Analysis
AMA Lab No.: M-7294
Client No.: Cream without Bonicel, Lot 28378

| Panelist ID Nos. | Baseline (px) | Day 14 (px) | Individual % Difference | Day 28 (px) | Individual % Difference |
|---|---|---|---|---|---|
| 56 0090 | 16669 | 14935 | −10.40% | 18640 | 11.82% |
| 52 3397 | 21205 | 21882 | 3.19% | 23500 | 10.82% |
| 62 9653 | 8919 | 9833 | 10.25% | 10280 | 15.26% |
| 58 6382 | 2866 | 3227 | 12.60% | 3052 | 6.49% |
| 50 7599 | 16966 | 21531 | 26.91% | 20282 | 19.54% |

| | | |
|---|---|---|
| Average % Difference: | 8.51% | 12.79% |
| Max % Difference: | −10.40% | 19.54% |
| p | 0.400 | 0.024* |
| t | 0.940 | 3.525* |

* Statistically Significant

A summary of the wrinkle and fine lines reduction analysis for each of the two groups (i.e., with and without Bonicel) is provided in Table 54 below.

TABLE 54
Reverse Photo Engineering - Wrinkle
and Fine Lines Reduction Analysis

| | |
|---|---|
| Day 14 | Day 28 |

Active Treatment Group
AMA Lab No.: M-7293
Client No.: Cream with Bonicel (bacillus ferment), Lot 28378

| | | |
|---|---|---|
| % Difference: | −56.97%* | −71.50%* |
| Max % Improvement: | −70.51% | −87.06% |

Placebo Group
AMA Lab No.: M-7294
Client No.: Cream without Bonicel, Lot 28378

| | | |
|---|---|---|
| % Difference: | 8.51% | 12.79%* |
| Max % Difference: | −10.40% | 19.54% |

*Statistically Significant

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A topical composition for the reduction of visible signs of aging comprising an anti-aging amount of an extracellular product of Bacillus coagulans and a dermatologically acceptable carrier.

2. The composition of claim 1, wherein said Bacillus coagulans is selected from the group consisting of GBI-30 strain (ATCC Designation Number PTA-6086), GBI-20 strain (ATCC Designation Number PTA-6085), and GBI-40 strain (ATCC Designation Number PTA-6087).

3. The composition of claim 1, wherein said extracellular product comprises a liquid culture supernatant.

4. The composition of claim 1, wherein said composition is in the form of an emulsion, a lotion, a cream, an oil, an ointment, a suspension, a gel, a dried powder, an aerosol powder, a scrub, a mask, an aerosol spray, a semi-solid formulation, a shampoo, and a conditioner.

5. The composition of claim 1, wherein said composition comprises at least 5% by volume of said extracellular product or at least 5% by weight of said extracellular product.

6. The composition of claim 1, wherein said composition further comprises from 0.1% to 10% by weight of a penetration enhancer selected from the group consisting of sulfoxides, alcohols, polyols, alkanes, fatty acids, esters, amines and amides, terpenes, surface-active agents, cyclodextrins, and mixtures thereof.

7. The composition of claim 1, wherein said extracellular product of Bacillus coagulans is dried.

8. The composition of claim 1, wherein said extracellular product of Bacillus coagulans is dried and reconstituted.

9. A method for the topical reduction of visible signs of aging in a subject comprising topically applying to affected skin a composition comprising an anti-aging amount of an extracellular product of Bacillus coagulans and a dermatologically acceptable carrier.

10. The method of claim 9, wherein said skin is not characterized by a pathologic microbial infection, wherein said pathologic microbial infection comprises an infection by a pathologic virus, yeast, fungus, or bacteria.

11. The method of claim 9, wherein said subject is identified as suffering from visible signs of aging or a predisposition thereto by detecting a sign or symptom selected from the group consisting of fine lines or wrinkles around the eye area, under-eye puffiness, dark under-eye circles, rough skin, reduced skin hydration/moisturization, and reduced skin elasticity.

12. The method of claim 9, wherein said extracellular product of Bacillus coagulans is dried and reconstituted.

13. The method of claim 9, wherein said extracellular product of Bacillus coagulans is lyophilized.

14. The method of claim 9, wherein skin pore size is decreased by at least 5%, wherein skin roughness is decreased by at least 5%, wherein skin redness is decreased by at least 5%, wherein hydration of said skin is improved by at least 5%, wherein elasticity of said skin is improved by at least 5%, wherein fine lines and wrinkles are reduced by at least 5%, wherein under eye puffiness is reduced by at least 5%, wherein under eye dark circles are reduced by at least 5%, or wherein skin inflammation is reduced by at least 5%, as compared to a pre-treatment baseline.

15. The method of claim 9, wherein said composition inhibits the growth of pathogenic bacteria, fungus, or yeast.

16. The method of claim 9, wherein said composition is administered once per day.

17. The method of claim 9, wherein said composition is administered for 30 days.

18. The method of claim 9, wherein said subject is a human.

19. A composition comprising an acellular culture supernatant of Bacillus coagulans in a eukaryotic tissue culture medium, wherein said composition is in the form of a dry powder.

20. The composition of claim 19, wherein said medium is serum free medium.

21. The composition of claim 19, wherein said medium comprises Roswell Park Memorial Institute (RPMI)-1640 medium, Dulbecco's modified eagle medium (DMEM), Eagle's minimal essential medium (EMEM), minimal essential medium (MEM), Iscove's modified Dulbecco's media (IMDM), Ham's medium, minimal essential medium alpha (AMEM), Glasgow minimal essential medium (GMEM), or Hank's balanced salt solution medium (HBSS).

22. A topical composition for the reduction of visible signs of aging comprising an anti-aging amount of an isolated Bacillus coagulans bacterium and a dermatologically acceptable carrier.

23. The composition of claim 22, wherein said isolated Bacillus coagulans bacterium is in the form of a spore.

24. The composition of claim 22, wherein said isolated Bacillus coagulans bacterium is in the form of a vegetative cell.

25. The composition of claim 22, wherein said composition is in the form of an emulsion, a lotion, a cream, an oil, an ointment, a suspension, a gel, a dried powder, an aerosol powder, a scrub, a mask, an aerosol spray, a semi-solid formulation, a shampoo, and a conditioner.

26. The composition of claim 22, wherein said composition comprises at least 5% by volume of said Bacillus coagulans bacterium or at least 5% by weight of said Bacillus coagulans bacterium.

27. The composition of claim 22, wherein said Bacillus coagulans bacterium is viable.

28. The composition of claim 22, wherein said Bacillus coagulans bacterium is non-viable, wherein said non-viable Bacillus coagulans bacterium is inactivated, irradiated, heat killed or dead.

29. The composition of claim 22, wherein said isolated Bacillus coagulans bacterium is selected from the group consisting of GBI-30 strain (ATCC Designation Number PTA-6086), GBI-20 strain (ATCC Designation Number PTA-6085), and GBI-40 strain (ATCC Designation Number PTA-6087).

30. The composition of claim 22, wherein the composition further comprises from 0.1% to 10% by weight of a penetration enhancer selected from the group consisting of sulfoxides, alcohols, polyols, alkanes, fatty acids, esters, amines and amides, terpenes, surface-active agents, cyclodextrins, and mixtures thereof.

31. The composition of claim 22, wherein said extracellular product of Bacillus coagulans is lyophilized.

32. A method for topically reducing visible signs of aging in a subject comprising topically applying to affected skin a composition comprising an anti-aging amount of an isolated Bacillus coagulans bacterium and a dermatologically acceptable carrier.

33. The method of claim 32, wherein said subject is identified as suffering from visible signs of aging or a predisposition thereto by detecting a sign or symptom selected from the group consisting of fine lines or wrinkles around the eye area, under-eye puffiness, dark under-eye circles, rough skin, reduced skin hydration/moisturization, and reduced skin elasticity

34. The method of claim 32, wherein said isolated Bacillus coagulans bacterium is selected from the group consisting of GBI-30 strain (ATCC Designation Number PTA-6086), GBI-20 strain (ATCC Designation Number PTA-6085), and GBI-40 strain (ATCC Designation Number PTA-6087).

35. The method of claim 32, wherein said skin is not characterized by a pathologic microbial infection, wherein said pathologic microbial infection comprises an infection by a pathologic virus, yeast, fungus, or bacteria.

36. The method of claim 32, wherein skin pore size is decreased by at least 5%, wherein skin roughness is decreased by at least 5%, wherein skin redness is decreased by at least 5%, wherein hydration of said skin is improved by at least 5%, wherein elasticity of said skin is improved by at least 5%, wherein fine lines and wrinkles are reduced by at least 5%, wherein under eye puffiness is reduced by at least 5%, wherein under eye dark circles are reduced by at least 5%, or wherein skin inflammation is reduced by at least 5%, as compared to a pre-treatment baseline.

37. The method of claim 32, wherein said composition is administered once per day.

38. The method of claim 32, wherein said composition is administered for 30 days.

39. The method of claim 32, wherein said subject is a human.

40. The method of claim 32, wherein said isolated Bacillus coagulans bacterium is in the form of a spore.

41. The method of claim 32, wherein said isolated Bacillus coagulans bacterium is in the form of a vegetative cell.

42. The method of claim 32, wherein said Bacillus coagulans bacterium is viable.

43. The method of claim 42, wherein said Bacillus coagulans bacterium is non-viable, wherein said non-viable Bacillus coagulans bacterium is inactivated, irradiated, heat killed or dead.

44. The method of claim 32, wherein said Bacillus coagulans bacterium is lyophilized.

45. A method for topically reducing visible signs of a skin disorder in a subject comprising topically applying to affected skin a composition comprising an isolated Bacillus coagulans bacterium or an extracellular product thereof and a dermatologically acceptable carrier.

46. The method of claim 45, wherein said skin disorder is acne.

47. The method of claim 45, wherein said Bacillus coagulans bacterium is lyophilized.

48. The method of claim 45, wherein said Bacillus coagulans extracellular product is dried and reconstituted.

49. The composition of claim 7, wherein said extracellular product of Bacillus coagulans is lyophilized, freeze-dried, vacuum dried, air dried, or dried by application of heat.
